(19) United States
(12) Patent Application Publication
(10) Pub. No.: US 2022/0288273 A1
(43) Pub. Date: Sep. 15, 2022

(54) OSTEOINDUCTIVE MODIFIED GELATIN HYDROGELS AND METHODS OF MAKING AND USING THE SAME

(71) Applicant: THE REGENTS OF THE UNIVERSITY OF CALIFORNIA, Oakland, CA (US)

(72) Inventors: Nasim Annabi, Los Angeles, CA (US); Ehsan Shirzaei Sani, Los Angeles, CA (US)

(21) Appl. No.: 17/618,390

(22) PCT Filed: Jun. 12, 2020
(86) PCT No.: PCT/US2020/037381

§ 371 Date: Dec. 10, 2021

Related U.S. Application Data
(60) Provisional application No. 62/860,939, filed on Jun. 13, 2019.

(51) Int. Cl. A61L 27/22; A61L 27/34; A61L 27/54; A61L 27/52; A61L 27/04

(52) U.S. Cl. CPC A61L 27/222; A61L 27/34; A61L 27/54; A61L 27/52; A61L 27/042; A61L 27/047; A61L 27/045; A61L 2430/12; A61L 2300/412; A61L 2300/404; A61L 2300/25; B82Y 5/00

(57) ABSTRACT

The present invention relates in part to a hydrogel composition and a method of fabricating the hydrogel composition comprising the steps of providing a solution comprising a polymer comprising crosslinkable groups; providing a solution comprising a crosslinking agent; mixing the solution comprising a polymer comprising crosslinkable groups and the solution comprising the crosslinking agent to form a combined solution; and crosslinking the combined solution. The invention also relates in part to a methods of treatment using the hydrogel composition.

### CROSS-REFERENCE TO RELATED APPLICATIONS

The present application claims priority to U.S. Provisional Patent Application Ser. No. 62/860,939, filed Jun. 13, 2019, which is incorporated by reference herein in its entirety.

### STATEMENT REGARDING FEDERALLY SPONSORED RESEARCH OR DEVELOPMENT

This invention was made with government support under Grant Numbers DE026914, EB023052, HL140618, awarded by the National Institutes of Health. The government has certain rights in the invention.

### BACKGROUND OF THE INVENTION

Placement of dental implants has become the standard of care for partial or fully edentulous patients. However, with an estimated 12 million implants placed worldwide each year, postoperative complications affecting the peri-implant tissues have emerged as a serious health concern (Verdugo, F. et al., Arch. Oral Biol., 2016, 64:9-50). As dental implants have become the standard of care for tooth replacement, the number of patients affected by peri-implant diseases (PIDs) is increasing (The American Academy of Periodontology (AAP), J. Periodontol., 2013, 84:436-443). According to their clinical manifestations, PIDs can be categorized in peri-implant mucositis (PIM) and peri-implantitis (PI) (Ata-Ali, J. et al., Open Dent. J., 2015, 9:393-395). PIM refers to a reversible inflammatory process that affects the soft tissues surrounding an implant, resulting in bleeding on gentle probing, and in some cases, suppuration, erythema, and swelling (Berglundh, T. et al., Journal of Clinical Periodontology, 2018, 45:S286-S291). The etiology of PIM is the bacterial accumulation and biofilm formation around the dental implant (Renvert, S. et al., Journal of Clinical Periodontology, 2018, 45:S278-S285). On the other hand, PI presents not only with inflammation of the soft tissues but is also accompanied by a progressive bone loss that could lead to implant failure (The American Academy of Periodontology (AAP), J. Periodontol., 2013, 84:436-443). Clinical data has shown that progression from PIM to PI is strongly associated with lack of preventive maintenance and thus, opportune treatment of PIM could prevent the progression to PI (Costa, F. O. et al., J. Clin. Periodontol., 2012, 39:173-181).

Current treatments against PIM are mainly aimed at eradicating subgingival dysbiosis and restoring homeostasis to microbial communities in the oral cavity (Frederic, L. J. et al., Materials (Basel) 2018, 11:1802). PIM can be treated with nonsurgical procedures, which include mechanical debridement, alone or in combination with local delivery of antibiotics such as Arestin (minocycline HCL), Elyzol® (metronidazole 25%), and Atridox® (doxycycline hyclate 10%) which can be injected directly into the sulcus or peri-implant pockets (Renvert, S. et al., J. Clin. Periodontol. 2008, 35:305-315; Mombelli, A. et al., Clinical Oral Implants Research, 2001, 12:287-294; Schenk, G. et al., Clinical Oral Implants Research, 1997, 8:427-433). However, because of their inability to efficiently antagonize the infection (Aljateeli, M. et al., Journal of Michigan Dental Association, 2013, 95:42-47; Renvert, S. et al., Journal of Clinical Periodontology, 2008, 35:305-315), the therapeutic efficacy of these approaches is limited (Grusovin, M. G. et al., Cochrane Database Syst. Rev. 2010; Suarez-Lopez Del Amo, F. et al., J. Oral Maxillofac. Res., 2016, 7:e13). Particularly, clinical data has shown that nonsurgical mechanical approaches, aimed at disinfection of the affected area, often fail due to recolonization of the periodontal or peri-implant pockets by pathogenic bacteria that perpetuate the disease (Frederic, L. J. et al., Materials (Basel) 2018, 11:1802; Derks, J. et al., J. Clin. Periodontol., 2015, 42 Suppl 16:S158-S171). Moreover, bacterial infection and the subsequent epithelial cell death lead to the release of inflammatory cytokines and chemotactic bacterial peptides, which attract migratory neutrophils that could worsen implant prognosis. This is mainly because neutrophil degranulation due to bacterial overload releases tissue-degrading enzymes into the gingival crevice that lead to further tissue trauma (Kinane, D. F., Periodontol. 2000, 2001, 25:8-20; Heitz-Mayfield, L. J. A. et al., J. Periodontol., 2018, 89 Suppl 1:S257-S266). As inflammation extends from the marginal gingiva into the supporting periodontal tissues, PIM could eventually progress to PI and lead to bone loss and implant failure. The administration of local and systemic antibiotics leads to different problems and may result in hypersensitivity reactions in allergic patients, as well as the development of antibiotic-resistant strains of pathogenic bacteria (Diz, P. et al., Journal of Dentistry, 2013, 41:195-206; Esposito, M., et al., Antibiotics to prevent complications following dental implant treatment. The Cochrane Library, 2003).

PI, the severe inflammatory process affecting the soft and hard tissues surrounding an implant, which is characterized by a progressive loss of the supporting bone (Poli, P. P. et al., J. Periodontol., 2013, 84:436-443), constitutes the leading cause of implant failure after osseointegration. Despite conflicting reports, recent clinical data indicates that PI could occur in up to 87.5% of patients (Papathanasiou, E. et al., J. Periodontol., 2016, 87:493-501). However, the lack of standard treatment protocols often leads to empirical selection of therapeutic strategies and marginally effective outcomes (Esposito, M. et al., Cochrane Database Syst. Rev., 2012, 1:CD004970). Microbial colonization in the form of dental plaque biofilms constitutes the main etiological factor of PI (Fu, J. H. et al., Dent. Clin. North Am., 2015, 59:951-980).

Treatment of PI is carried out primarily via surgical approaches with/without local or systemic administration of antibiotics. However, as for PIM, conventional antibiotics have shown limited efficacy for treatment of PI (Aljateeli, M. et al., Journal of Michigan Dental Association, 2013, 95:42-47; Renvert, S. et al., Journal of Clinical Periodontology, 2008, 35:305-315), and administration to patients that are allergic to them could lead to severe hypersensitivity reactions (Diz, P. et al., Journal of Dentistry, 2013, 41:195-206; Esposito, M., et al., Antibiotics to prevent complications following dental implant treatment. The Cochrane Library, 2003). Moreover, the over prescription and misuse of antibiotics have led to an escalating increase in bacterial resistance (Ageitos, J. M. et al., Biochem. Pharmacol., 2016, 133:117-138). Twelve treatment strategies have been evaluated for PI management, but the available evidence did not determine the most effective way to treat PI. There were indications that, locally-applied antibiotics (8.5 per cent doxycycline hyclate) or augmentation with an animal-derived bone substitute (Bio-Oss™) significantly decreased probing pocket depth (PPD) and probing attachment level (PAL). There are also some other bone graft products such as INFUSE®, PROGENIX®, Grafton DBM and MinerOss, but none of them is specifically designed for treatment of PI, nor has antimicrobial properties. Most of the trials that tested more complex and expensive therapies did not show any statistically or clinically significant advantages over deep mechanical cleaning (Inc., i.R., US Dental Bone Graft Substitutes and other Biomaterials Market. 2015: iData Research Inc.). INFUSE®, a commercially available product for bone regeneration, based on combination of human recombinant bone morphogenetic protein 2 (hrBMP2) and collagen, has also been proposed for implant re-osseointegration (Hanisch, O. et al., Int. J. Oral Maxillofac. Implants, 1997, 12:604-610). Yet, the uncontrolled release rate of the growth factor (Tevlin, R. et al., J. Dent. Res., 2014, 93:1187-1195) and the potentially harmful side effects associated with hrBMP2 (Woo, E. J., J. Oral Maxillofac. Surg., 2012, 70:765-767; Mesfin, A. et al., J. Bone Joint Surg. Am., 2013, 95:1546-1553) severely limit its application in PI regenerative procedures. Currently, there are no commercially available products that combine high adhesion to soft and hard oral tissues, and antimicrobial and osteoinductive properties; therefore, clinical management of PI remains challenging.

As the number of dental implants being placed has continue to increase worldwide, it is predicted that PIDs will become one of the most prominent dental diseases of the future (Levin, L., Quintessence Int. 2013, 44:643). Therefore, therapeutic strategies that efficiently isolate the affected area to prevent the infiltration of bacteria and other unwanted cells and promote healing around dental implants while also enabling the growth of bone-competent cells (i.e., compartmentalized tissue healing) could improve the clinical outcome of patients with PIDs (Gottlow, J. et al., J. Clin. Periodontol., 1984, 11:494-503; Nyman, S., J. Clin. Periodontol., 1991, 18:494-498). Periodontal regeneration requires the hierarchical and coordinated response of a variety of soft and hard tissues (i.e., periodontal ligament, gingiva, cementum, and bone) during the wound healing process (Ivanovski, S. et al., J. Dent. Res., 2014, 93:1212-1221). In recent years, clinical evidence has shown that treatment options based on resorbable and non-resorbable membranes could be used for guided tissue regeneration of the periodontal tissues affected by PIDs (Siaili, M. et al., Saudi Dent. J., 2018, 30:26-37). Current third-generation membranes are developed not only to act as passive barriers but also as delivery vehicles for the release of specific antibiotics and growth factors (Larsson, L. et al., J. Dent. Res., 2016, 95:255-266; Sam, G. et al., J. Clin. Diagn. Res., 2014, 8:ZE14-17). Moreover, local delivery yields higher local concentrations of the therapeutic agents, which increases the effectiveness at the site and decreases the risk of systemic side effects. However, several limitations remain pertaining to the unpredictability of the efficacy of these treatments and the need for the delivery multiple biological mediators to promote tissue regeneration (Sculean, A. et al., Periodontol 2000, 2015, 68:182-216; Kao, R. T. et al., J. Periodontol., 2015, 86:S77-S104).

There is a need in the art for antimicrobial and osteoinductive hydrogel adhesive compositions that, by adhering to both hard (titanium implants) and soft tissue (gingiva) surfaces, will allow compartmentalized tissue healing and foster tissue regeneration and for methods to use such compositions to treat PIDs. The present invention satisfies this unmet need.

### SUMMARY OF THE INVENTION

In one aspect, the present invention relates to a hydrogel composition comprising crosslinked gelatin, a crosslinking agent, an antimicrobial agent, and an osteoinductive agent. In one embodiment, the crosslinked gelatin comprises crosslinkable groups selected from the group consisting of: methyl acrylate, ethyl acrylate, propyl acrylate, methyl methacrylate, ethyl methacrylate, methacryloyl, catechol, ethylene oxide, propylene oxide, and combinations thereof. In one embodiment, the crosslinking agent comprises a photoinitiator selected from the group consisting of: 2-hydroxy-4′-(2-hydroxyethoxy)-2-methylpropiophenone; lithium phenyl-2,4,6-trimethylbenzoylphosphinate; 2,2-diethoxyacetophenone; triethanolamine; N-vinyl caprolactam; benzophenone; Eosin Y; and combinations thereof. In one embodiment, the crosslinking agent comprises a metal²⁺ or metal³⁺ ion selected from the group consisting of: Fe²⁺, Fe³⁺, Ni²⁺, Zn²⁺, Cu²⁺, Ag²⁺, Au³⁺, Co³⁺, Cr²⁺, Cr³⁺, Cd²⁺, Mn²⁺, Mg²⁺, Pd²⁺, Pt²⁺, Al³⁺, and combinations thereof. In one embodiment, the antimicrobial agent comprises an antimicrobial peptide. In one embodiment, the osteoinductive agent is selected from the group consisting of: silicate nanoparticles, calcium phosphate, calcium sulfate, bioglass, hydroxyapatite, demineralized bone matrix (DBM), and combinations thereof. In one embodiment, the osteoinductive agent comprises silicate nanoparticles and wherein the silicate nanoparticles comprise laponite nanoparticles.

In another aspect, the present invention relates to a method of making a hydrogel, the method comprising: providing a solution comprising gelatin modified with crosslinkable groups; providing a solution comprising a crosslinking agent; mixing the solution comprising the gelatin modified with crosslinkable groups and the solution comprising the crosslinking agent to form a combined solution; and crosslinking the combined solution. In one embodiment, the step of providing a solution comprising a crosslinking agent further comprises the step of adding an antimicrobial agent to the solution. In one embodiment, the step of mixing the solution comprising the gelatin modified with crosslinkable groups and the solution comprising the crosslinking agent to form a combined solution further comprises the step of adding an osteoinductive agent to the combined solution. In one embodiment, the gelatin modified with crosslinkable groups is made by a method comprising the steps of: providing a solution comprising gelatin; and reacting the solution comprising gelatin with a compound comprising crosslinkable groups. In one embodiment, the gelatin modified with crosslinkable groups is selected from the group consisting of gelatin modified with methacryloyl groups (GelMA), gelatin modified with catechol groups (GelMAC), and gelatin modified with both methacryloyl groups and catechol groups. In one embodiment, the crosslinking agent is selected from the group consisting of 2-hydroxy-4′-(2-hydroxyethoxy)-2-methylpropiophenone; lithium phenyl-2,4,6-trimethylbenzoylphosphinate; 2,2-diethoxyacetophenone; triethanolamine; N-vinyl caprolactam; benzophenone; Eosin Y; Fe²⁺; Fe³⁺; Ni²⁺; Zn²⁻; Cu²⁺; Ag²⁺; Au³⁺; Co²⁺; Co³⁺; Cr²⁺; Cr³⁺; Cd²⁺; Mn²⁺; Mg²⁺; Pd²⁻; Pt²⁺; Al³⁺; and combinations thereof. In one embodiment, the antimicrobial agent comprises an antimicrobial peptide.

In another aspect, the present invention relates to a method of inhibiting microbial growth at the site of a dental implant, the method comprising: applying a solution comprising a hydrogel precursor and an antimicrobial agent to one or more surfaces of a dental implant in the subject's mouth to form a coating; and crosslinking the coating to form a hydrogel. In one embodiment, the step of crosslinking the coating to form a hydrogel further comprises the step of crosslinking the coating by irradiating the coating with visible light. In one embodiment, the step of crosslinking the coating to form a hydrogel further comprises the step of adhering the hydrogel to the dental implant in the subject's mouth. In one embodiment, the microbial growth comprises microbial growth associated with peri-implant or periodontal diseases. In one embodiment, the step of adhering the hydrogel to the dental implant in the subject's mouth further comprises the step of promoting bone growth at the site of the implant. In one embodiment, the microbial growth is associated with bacteria selected from the group consisting of: Eubacterium nodatum, E. brachy, E. saphenum, Filifactor alocis, Slackia exigua, Parascardovia denticolens, Prevotella intermedia, Fusobacterium nucleatum, Porphyromonas gingivalis, Centipeda periodontii, Parvimonas micra, Prevotella buccae, Prevotella oralis, Prevotella melaninogenica, Prevotella denticola, Prevotella nigrescens, Tannerella forsythia, Treponema denticola, and combinations thereof.

In another aspect, the present invention relates to a method of promoting bone regrowth in a subject's mouth, the method comprising: applying a solution comprising a hydrogel precursor and an antimicrobial agent to one or more defects in the subject's mandible or mouth as a bone graft; and crosslinking the solution.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 is a flowchart of an exemplary method for the fabrication of a hydrogel of the present invention.

FIG. 2 depicts the synthesis and photocrosslinking process of bioadhesive hydrogels.

FIG. 3, comprising FIGS. 3A-E, depicts the physical characterization of the of the adhesive hydrogels produced by using 7% and 15% (w/v) total polymer concentration with and without AMP. FIG. 3A depicts the elastic and compressive of the hydrogels. FIG. 3B depicts the extensibility of the hydrogels. FIG. 3C depicts the ultimate stress of the hydrogels.

FIG. 3D depicts the in vitro degradation properties of the hydrogels in 20 μg/ml collagenase type II solution in Dulbecco's phosphate buffered saline (DPBS). FIG. 3E depicts the swelling ratios of the hydrogels in DPBS. Data in all figures are represented as mean+SD (*p<0.05, **p<0.01, ***p<0.001, ****p<0.0001 and n≥5).

FIG. 4, comprising FIGS. 4A-B, depicts the in vivo assessment of biodegradation of bioadhesive hydrogels using a subcutaneous implantation model in rats.

FIG. 4A depicts a histological evaluation (H&E staining) of 15% GelAMP bioadhesive after implantation in a rat subcutaneous model at day 7 post implantation. FIG. 4B depicts an H&E staining of 15% GelAMP bioadhesive after implantation in a rat subcutaneous model at day 56 post implantation.

FIG. 5, comprising FIGS. 5A-E, depicts the in vitro and ex vivo adhesion properties of GelAMP (gelatin methacryloyl with AMP) hydrogels. FIG. 5A depicts a schematic of the in vitro lap shear test based on a modified ASTM standard (F2255-05), using titanium as a substrate. FIG. 5B depicts the in vitro lap shear strength of the bioadhesive hydrogels at 7% and 15% polymer concentration and a commercially available adhesive (CoSEAL™). FIG. 5C depicts representative images of a wound closure test using pig gingiva tissue based on ASTM standard test (F2458-05). FIG. 5D depicts the adhesion strength of bioadhesive hydrogels and a commercially available adhesive (CoSEAL™) to porcine gingiva.

FIG. 5E depicts the adhesion strength of bioadhesive hydrogels and a commercially available adhesive (CoSEAL™) to porcine skin. Data in all figures are represented as mean+SD (**p<0.01, ***p<0.001, ****p<0.0001, n=5).

FIG. 6 depicts the in vitro burst pressure test of bioadhesive hydrogels and a commercially available adhesive (CoSEAL™) on porcine intestine tissue. Data are represented as mean±SD (*p<0.05, **p<0.01, ***p<0.001, ****p<0.0001, n=5).

FIG. 7, comprising FIGS. 7A-D, depicts the in vitro antibacterial properties of bioadhesive hydrogels against P. gingivalis. FIG. 7A depicts representative images of P. gingivalis colonies grown on blood agar plates for bioadhesives with and without AMP (Dilution 1, 3 and 4 represent 1-, 3- and 4-logarithmic dilutions respectively). FIG. 7B depicts the quantification of colony forming units (CFUs) for bioadhesive hydrogels with AMP (0.2% (w/v) or 1.34 mM) and without AMP, seeded with P. gingivalis bacteria (day 4). Representative scanning electron microscope (SEM) images of P. gingivalis colonization on bioadhesive hydrogels containing (C) 0% and (D) 0.2% AMP. Clusters of bacteria are shown with yellow arrows. (***p<0.001 and ****p<0.0001).

FIG. 8, comprising FIGS. 8A-F, depicts the in vitro 3D encapsulation of W-20-17 cells and mouse calvarial bone sutures inside adhesive hydrogels. FIG. 8A depicts representative live/dead images of W-20-17 cells encapsulated within bioadhesives hydrogels with and without AMP after 1 and 5 days. FIG. 8B depicts representative phalloidin (green)/DAPI (blue) stained images of cell-laden bioadhesive with and without AMP after 1 and 5 days. FIG. 8C depicts the quantification of viability of W-20-17 incorporated within hydrogels without (control) and with AMP (GelAMP) using live/dead assays on days 1, 3, and 5 post encapsulation. FIG. 8D depicts the quantification of metabolic activity of W-20-17 cells encapsulated in hydrogels after 1, 3, and 5 days. FIG. 8E depicts a schematic diagram of the extraction and encapsulation of mouse calvarial bone sutures in 3D hydrogel network. FIG. 8F depicts representative images of calvarial bone sutures encapsulated within 7% and 15% (w/v) bioadhesives to visualize growth and diffusion of cells at days 10, 20, and 30 post encapsulation. FIG. 8G depicts the quantification of metabolic activity of migratory stromal cells from encapsulated bone sutures. Data in all figures are from hydrogels formed at 120 sec visible light exposure time (** p<0.01, *** p<0.001), **** p<0.0001).

FIG. 9, comprising FIGS. 9A-F, depicts the in vivo evaluation of bioadhesive hydrogels using a mouse calvarial defect model. FIG. 9A depicts a schematic diagram of in situ application of bioadhesive hydrogels in a mouse calvarial defect model. FIG. 9B depicts the results after 7% and 15% bioadhesive hydrogels were delivered to artificially created bone defects in mouse calvaria (yellow arrowheads), and photopolymerized for 1 min using a commercially available dental curing light. 7 and 14 days after implantation, samples remained in place, without any sign of detachment. FIG. 9C depicts the histological evaluation (H&E staining) of the 15% (w/v) bioadhesives at day 0 post implantation. FIG. 9D depicts a representative H&E image for 7% (w/v) bioadhesive treatment. FIG. 9E depicts a representative H&E image for 15% (w/v) bioadhesive treatment. FIG. 9F depicts an untreated sample after 42 days post implantation.

FIG. 10, comprising FIGS. 10A-B, depicts the in vivo evaluation of bioadhesive hydrogels using a calvarial defect model in mice. FIG. 10A depicts the results after 7% and 15% bioadhesives were delivered to artificially created bone defects in mouse calvaria (yellow arrowheads), and photopolymerized for 1 min using a commercially available dental curing light. FIG. 10B depicts histological evaluation (H&E staining) of the 7% and 15% (w/v) hydrogels at days 0, 7, and 14 post implantation.

FIG. 11, comprising FIGS. 11A-C, depicts the quantitative evaluation of new bone formation using μCT (micro-CT; Micro computed tomography) analysis. FIG. 11A depicts representative micro-CT images for an untreated defect, and defects treated with 7% and 15% bioadhesives on days 28 and 42 post-implantation FIG. 11B depicts the quantitative analysis of bone surface area. FIG. 11C depicts the quantitative analysis of bone volume. Data in all figures are represented as mean±SD (*p<0.1, **p<0.01, ***p<0.001, ****p<0.0001, n=5).

FIG. 12 shows the synthesis process of GelMA/AMP/SN bioadhesives (SN refers to silicate nanoparticles).

FIG. 13, comprising FIGS. 13A-E, depicts the physical characterization of bioadhesive hydrogels. FIG. 13A depicts the compressive modulus of the adhesive hydrogels produced by using 15% (w/v) total polymer concentration and different SN content. FIG. 13B depicts the elastic modulus of the adhesive hydrogels produced by using 15% (w/v) total polymer concentration and different SN content. FIG. 13C depicts the extensibility of the adhesive hydrogels produced by using 15% (w/v) total polymer concentration and different SN content. FIG. 13D depicts the in vitro degradation properties in Dulbecco's phosphate buffered saline (DPBS) for 15% (w/v) adhesive hydrogels containing various concentrations of SN. FIG. 13E depicts the swelling ratios in DPBS for 15% (w/v) adhesive hydrogels containing various concentrations of SN with and without AMP. Data in all of FIGS. 13A-E are represented as mean±SD (*p<0.05, **p<0.01, ***p<0.001, ****p<0.0001 and n≥5).

FIG. 14, comprising FIGS. 14A-B, depicts the in vitro and ex vivo adhesion properties of adhesives hydrogels. FIG. 14A depicts the adhesion strength of bioadhesive hydrogels and a commercially available adhesive (CoSEAL™) to porcine gingiva tissue based on ASTM standard wound closure test (F2458-05). FIG. 14B depicts the in vitro lap shear strength of the bioadhesive hydrogels and a commercially available adhesive (CoSEAL™) on titanium substrate, based on a modified ASTM standard (F2255-05). Data in FIGS. 15A-B are represented as mean±SD (**p<0.01, ***p<0.001, ****p<0.0001, n=5).

FIG. 15, comprising FIGS. 15A-F, depicts the in vitro antibacterial properties of bioadhesive hydrogels against different aerobic/anerobic and G+/− bacteria. FIG. 15A depicts the quantification of optical density (OD) growth of P. gingivalis bacteria cultured in different bioadhesive solutions with and without AMP, and a commercial antibiotic (SMZ-TMP) as control. FIG. 15B depicts the quantification of colony forming units (CFUs) for bioadhesive hydrogels with and without AMP (0, 0.1, 0.2, and 0.4% (w/v)), seeded with a 3-logarithmic dilution of P. gingivalis bacteria (day 4). FIG. 15C depicts the quantification of colony forming units (CFUs) for bioadhesive hydrogels with and without AMP (0, 0.1, 0.2, and 0.4% (w/v)), seeded with a 4-logarithmic dilution of P. gingivalis bacteria (day 4). FIG. 15D depicts the quantification of colony forming units for bioadhesive hydrogels without and with 0.4% (w/v) AMP, seeded with MDR e. coli. FIG. 15E depicts the quantification of colony forming units for bioadhesive hydrogels without and with 0.4% (w/v) AMP, seeded with MRSA. FIG. 17F depicts the quantification of colony forming units for bioadhesive hydrogels without and with 0.4% (w/v) AMP, seeded with Staphylococcus aureus. Data in FIGS. 17A-F are represented as mean SD (*p<0.05, **p<0.01 and ****p<0.0001).

FIG. 16 depicts representative images of bacterial colonies grown on agar plates for bioadhesives with and without AMP and SN (Dilution 1, 2, 3 and 4 represent 1-, 3- and 4-logarithmic dilutions respectively).

FIG. 17, comprising FIGS. 17A-D, depicts the in vitro cytocompatibility of the bioadhesive using hMSCs. FIG. 17A depicts representative live/dead images of hMSCs seeded on the surface of antimicrobial bioadhesive hydrogels with different SN content after 1 and 5 days. FIG. 17B depicts representative phalloidin (green)/DAPI (blue) stained images of cells seeded on bioadhesives after 1 and 5 days. FIG. 17C depicts the quantification of viability of cells seeded on the hydrogels using live/dead assays on days 1, 3, and 5 post encapsulation. FIG. 17D depicts the quantification of metabolic activity of hMSCs seeded on the surface of hydrogels after 1, 3, and 5 days.

FIG. 18 comprising FIGS. 18A-D, depicts the in vitro cytocompatibility and osteogenic differentiation of hMSCs. FIG. 18A depicts representative fluorescent images of calcian AM (green: live) and ethidium homodimer I (red: dead) for hMSCs seeded on the surface of well-plate (control), bioadhesive hydrogels containing AMP, with and without SNs, and Bio-OSS bone graft (control) after 7 and 15 days. FIG. 18B depicts representative images of Alcian Blue staining for hMSCs seeded on the surface of well-plate (control), bioadhesive hydrogels containing AMP, with and without SNs, and Bio-OSS bone graft after 7 and 15 days. FIG. 18C depicts Alizarin Red staining for hMSCs seeded on the surface of well-plate (control), bioadhesive hydrogels containing AMP, with and without SNs, and Bio-OSS bone graft after 7 and 15 days. FIG. 18D depicts Von Kossa staining for hMSCs seeded on the surface of well-plate (control), bioadhesive hydrogels containing AMP, with and without SNs, and Bio-OSS bone graft after 7 and 15 days.

FIG. 19, comprising FIGS. 19A-B, depicts RT-PCR analysis of in vitro differentiation of hMSCs seeded on bioadhesive hydrogels. FIG. 19A depicts a chart showing the quantification of gene expression for hMSCs seeded on bioadhesive hydrogels formed with different concentrations of SN and compared to BMP2 treated cells as control. FIG. 19B depicts the data showing the quantification of gene expression for hMSCs seeded on bioadhesive hydrogels formed with different concentrations of SN and compared to BMP2 treated cells as control.

FIG. 20, comprising FIGS. 20A-C, depicts the in vitro differentiation of w-20-17 cells seeded on bioadhesive hydrogels. FIG. 23A depicts representative images of Alizarin red staining for w-20-17 cells seeded on the bioadhesive hydrogels containing different concentrations of SN. FIG. 20B depicts the quantification of Ca′ deposition for w-20-17 cells seeded on the bioadhesive hydrogels containing different concentrations of SN. FIG. 20C depicts the quantification of alkaline phosphatase assays for w-20-17 cells seeded on the bioadhesive hydrogels containing different concentrations of SN.

FIG. 21, comprising FIGS. 21A-C, depicts the in vivo biocompatibility and biodegradation of hydrogels in a rat subcutaneous implantation model. FIG. 21A depicts representative H&E) for bioadhesives containing 0, 1000, and 10000 μg/ml SN for up to 56 days after subcutaneous implantation in rats. FIG. 21B depicts the in vivo biocompatibility of composite hydrogels using a rat subcutaneous model (CD3 staining). FIG. 21C depicts the in vivo biocompatibility of composite hydrogels using a rat subcutaneous model (CD68 staining).

FIG. 22, comprising FIGS. 22A-B, depicts in vivo evaluation of the bioadhesive hydrogels in a critical sized mandibular bone defect in a miniature pig model.

FIG. 22A depicts representative CT images for bioadhesive hydrogels and Bio-OSS bone graft (control) after application in a large defect in miniature pig mandible at day 0 post application.

FIG. 22B depicts representative CT images for bioadhesive hydrogels and Bio-OSS bone graft at day 60 post application.

FIG. 23 depicts an outline of the study to test the efficacy of the adhesives for PI treatment in a minipig model.

FIG. 24 depicts the in vivo application of the adhesives for treatment of large mandibular bone defects in minipigs.

FIG. 25 depicts the in vivo application of bioadhesive hydrogels and Bio-Oss commercial bone graft in a critical sized bone defect model in miniature pigs.

FIG. 26, comprising FIGS. 26A-B depicts studies performed on pig mandibles. FIG. 26A depicts representative images of tooth extraction process and closure of the wound in miniature pigs. FIG. 26B depicts representative CT images of the pig mandible, showing the area related to extracted teeth after 2 months healing. A tooth regrowth was observed in one defect site.

FIG. 27, comprising FIGS. 27A-B, depicts implant placement in the pig mandible. FIG. 27A depicts representative images of secondary tooth extraction process, implant placement, and closure of the wound in miniature pigs. FIG. 27B depicts representative CT images of the pig mandible, showing the implants after 2 months healing (two months after implant placement).

FIG. 28 depicts representative images of ligature and implant abutment placement in miniature pigs, two months after implant placement. Two silk ligatures were used per implant to induce peri-implantitis through bacterial accumulation.

FIG. 29 depicts representative photographic and CT images of the implants 3 months after ligature placement. A significant bone loss was observed around the implants.

FIG. 30 depicts representative images of implants with ligature and high plaque index, measurement of clinical parameters, mechanical debridement process, grafting with bioadhesive hydrogels, and closure of the wound in miniature pigs.

FIG. 31, comprising FIGS. 31A-B, depicts representative images of peri-implant defects after treatment. FIG. 31A depicts the defects after treatment with bioadhesive hydrogels. FIG. 31B depicts the defects after treatment with Dynablast, a commercial bone graft as control.

FIG. 32, comprising FIGS. 32A-B, depicts peri-implant prosthetic parameters. FIG. 32A depicts the total changes in probing pocket depth (PD) values for the implants treated with bioadhesive hydrogels, and Dynablast and untreated controls. FIG. 32B depicts the change in straight buccal changes probing pocket depth (PD) values for the implants treated with bioadhesive hydrogels, and Dynablast and untreated controls. Data in both FIGS. 32A-B are represented as mean+SD (*p<0.05, n≥3).

FIG. 33, comprising FIGS. 33A-D, depicts an analysis of bone regeneration and quality. FIG. 33A depicts micro computed tomography (μ-CT) images for the implants treated with bioadhesive hydrogels, and Dynablast and untreated controls at different angles. FIG. 33B depicts changes in total linear bone height calculated from CT images. FIG. 33C depicts bone volume fraction (BV/TV) for all the samples, calculated from μ-CT images. FIG. 36D depicts done surface density (BS/BV) for all the samples, calculated from μ-CT images. Data in all of FIGS. 33A-D are represented as mean±SD (*p<0.05, **p<0.01, ***p<0.001, n≥3).

FIG. 34 depicts the synthesis process of the wet tissue bioadhesives by conjugation of dopamine to gelatin backbone and further methacryloyl functionalization of the polymer.

FIG. 35, comprising FIGS. 35A-D, depicts the physical characterization of the GelMAC bioadhesive. FIG. 35A depicts the elastic modulus of a GelMAC wet tissue bioadhesive hydrogel. FIG. 35B depicts the compressive modulus of a GelMAC wet tissue bioadhesive hydrogel. FIG. 35C depicts the ultimate stress of a GelMAC wet tissue bioadhesive hydrogel. FIG. 35D depicts the extensibility of of a GelMAC wet tissue bioadhesive hydrogel.

FIG. 36, comprising FIGS. 36A-B, depicts in vitro adhesion properties of the bioadhesive hydrogels. FIG. 36A depicts a burst pressure test. FIG. 36B depicts a wound closure test.

FIG. 37, comprising FIGS. 37A-D, depicts in vitro cytocompatibility of the bioadhesive hydrogels. FIG. 37A shows the method of 3D cell encapsulation in wet tissue adhesives. FIG. 37B depicts the quantification of viability of the cells encapsulated within the adhesive hydrogels. FIG. 37C depicts the metabolic activity of the cells encapsulated within the adhesive hydrogels. FIG. 37D depicts the representative images of Live/Dead assay for the cells encapsulated within the wet tissue adhesives.

FIG. 38, comprising FIGS. 38A-D, depicts in vivo biodegradation and biocompatibility of composite hydrogels using a rat subcutaneous model (H&E staining). FIG. 38A depicts the biodegradation of wet tissue bioadhesives based on dry weight. FIG. 38B depicts the biodegradation of wet tissue bioadhesives based on wet weight. FIG. 38C represents a schematic of the location of the implanted samples in the rat subcutaneous pocket. FIG. 38D depicts representative H&E stained images from the cross sections of wet tissue bioadhesives explanted at days 7, 28, and 56.

FIG. 39, comprising FIGS. 39A-B, depicts in vivo biocompatibility of composite hydrogels using a rat subcutaneous model (immunohistochemical analysis). FIG. 39A depicts immunofluorescent analysis of subcutaneously implanted wet tissue bioadhesive hydrogels, explanted at day 7, and day 28. The samples were stained for CD206 (M2 macrophages), and F4/80 (total macrophages). FIG. 39B depicts quantification of macrophage infiltration based on immunofluorescent analysis of subcutaneously implanted wet tissue bioadhesive hydrogels, explanted at days 7, 28, and 56.

FIG. 40, comprising FIGS. 40A-D, depicts the hemostatic properties of the bioadhesive. FIG. 40A depicts the time-dependent clot formation of GelMA, GelMAC, GelMA-Fe, and GelMAC-Fe hydrogels compared with untreated blood (negative control) and SURGICEL® absorbable hemostat (positive control). FIG. 40B depicts the quantitative clot formation time of GelMA, GelMAC, GelMA-Fe, and GelMAC-Fe hydrogels compared with untreated blood (negative control) and SURGICEL® absorbable hemostat (positive control). FIG. 40C depicts the absorbance at 405 nm wavelength performed on clotted samples at various time points of 7, 12, 16, and 20 minutes for GelMA, GelMAC, GelMA-Fe, and GelMAC-Fe hydrogels compared with untreated blood (negative control) and SURGICEL® absorbable hemostat (positive control). FIG. 40D depicts the clot weight collected at a 16-minute time point for GelMA, GelMAC, GelMA-Fe, and GelMAC-Fe hydrogels compared with untreated blood (negative control) and SURGICEL® absorbable hemostat (positive control). (*p<0.05, ***p<0.001, ****p<0.0001 and n=4).

### DETAILED DESCRIPTION

In one aspect, the present invention relates to a hydrogel precursor composition. In another aspect, the present invention relates to a hydrogel comprising an antimicrobial agent, an osteoinductive agent, or both an antimicrobial and an osteoinductive agent. The present invention further relates to a method of making the hydrogel and a method of using the hydrogel precursor in conjunction with a dental implant to prevent/reduce microbial growth. In some embodiments, the present invention relates to using the hydrogel precursor in conjunction with a dental implant to promote bone growth.

### Definitions

Unless defined otherwise, all technical and scientific terms used herein generally have the same meaning as commonly understood by one of ordinary skill in the art to which this invention belongs. As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, the term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, ±5%, ±1%, ±0.1% from the specified value, as such variations are appropriate to perform the disclosed methods.

“Growth factor” refers to a substance that is effective to promote the growth of cells. Growth factors include, but are not limited to, basic fibroblast growth factor (bFGF), acidic fibroblast growth factor (aFGF), epidermal growth factor (EGF), insulin-like growth factor-I (IGF-T), insulin-like growth factor-II (IGF-II), platelet-derived growth factor-AB (PDGF), vascular endothelial cell growth factor (VEGF), activin-A, bone morphogenic proteins (BMPs), insulin, cytokines, chemokines, morphogens, neutralizing antibodies, other proteins, and small molecules.

“Hydrogel” refers to a water-insoluble and water-swellable cross-linked polymer. An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

A “therapeutic” agent is an agent administered to a subject who exhibits signs or symptoms of a disease or disorder, for the purpose of diminishing or eliminating those signs or symptoms.

As used herein, to “treat” means reducing the frequency with which symptoms of a disease, defect, disorder, or adverse condition, and the like, are experienced by a patient.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The present invention provides compositions for preventing or treating microbial growth. In one embodiment the composition is a hydrogel precursor composition. In one embodiment, the hydrogel precursor composition comprises a polymer comprising crosslinkable groups. In one embodiment, the polymer comprises gelatin. In one embodiment, the hydrogel precursor comprises a polymer comprising crosslinkable groups, and one or more crosslinking agents. In one embodiment, the hydrogel precursor composition comprises gelatin modified with crosslinkable groups, and one or more crosslinking agents. In one embodiment, the hydrogel precursor comprises one or more therapeutic agents. In one embodiment, the hydrogel precursor comprises an antimicrobial therapeutic agent. In one embodiment, the hydrogel precursor comprises an osteoinductive therapeutic agent. In one embodiment, the hydrogel precursor comprises one or more growth factors as therapeutic agents.

In one embodiment, the invention provides a hydrogel. In one embodiment, the hydrogel comprises a polymer that has been crosslinked by one or more crosslinking agents. In one embodiment, the hydrogel comprises gelatin that has been crosslinked by one or more crosslinking agents. In one embodiment, the hydrogel comprises one or more therapeutic agents. In one embodiment, the hydrogel comprises an antimicrobial therapeutic agent. In one embodiment, the hydrogel comprises an osteoinductive therapeutic agent. In one embodiment, the hydrogel comprises one or more growth factors as therapeutic agents.

### Hydrogel Precursor Compositions

In one aspect, the invention relates to a hydrogel precursor composition. In one embodiment, the hydrogel precursor composition comprises a polymer comprising crosslinkable groups, and one or more crosslinking agents. In one embodiment, the hydrogel precursor composition comprises a natural polymer modified with crosslinkable groups. Exemplary naturally occurring polymers include, but are not limited to, collagen, chitosan, alginate, hyaluronic acid, and gelatin. In one embodiment, the hydrogel precursor composition comprises a synthetic polymer comprising crosslinkable groups. The synthetic polymer can be any synthetic polymer known to a person of skill in the art. Exemplary synthetic polymers include, but are not limited to, polypropylene glycol, polyethylene glycol, polypropylene, polyvinyl chloride, polystyrene, nylon 6, nylon 6,6, thermoplastic polyurethane, and polytetrafluoroethylene. In one embodiment, the hydrogel precursor composition comprises gelatin modified with crosslinkable groups, and one or more crosslinking agents.

The polymer can comprise any crosslinkable groups known to a person of skill in the art. Exemplary crosslinkable groups include, but are not limited to, methyl acrylate, ethyl acrylate, propyl acrylate, methyl methacrylate, ethyl methacrylate, methacryloyl, catechol, ethylene oxide, and propylene oxide. In one embodiment, the crosslinkable groups comprise methacryloyl groups. In one embodiment, the crosslinkable groups comprise catechol groups. In one embodiment, the crosslinkable groups comprise both methacryloyl groups and catechol groups.

In one embodiment, the hydrogel precursor composition comprises between about 1% and about 90% (w/v) of a polymer comprising crosslinkable groups. In one embodiment, the hydrogel precursor composition comprises between about 1% and about 85% (w/v) of a polymer comprising crosslinkable groups. In one embodiment, the hydrogel precursor composition comprises between about 1% and about 80% (w/v) of a polymer comprising crosslinkable groups. In one embodiment, the hydrogel precursor composition comprises between about 1% and about 75% (w/v) of a polymer comprising crosslinkable groups. In one embodiment, the hydrogel precursor composition comprises between about 1% and about 70% (w/v) of a polymer comprising crosslinkable groups. In one embodiment, the hydrogel precursor composition comprises between about 1% and about 65% (w/v) of a polymer comprising crosslinkable groups. In one embodiment, the hydrogel precursor composition comprises between about 1% and about 60% (w/v) of a polymer comprising crosslinkable groups. In one embodiment, the hydrogel precursor composition comprises between about 1% and about 55% (w/v) of a polymer comprising crosslinkable groups. In one embodiment, the hydrogel precursor composition comprises between about 1% and about 50% (w/v) of a polymer comprising crosslinkable groups. In one embodiment, the hydrogel precursor composition comprises between about 1% and about 45% (w/v) of a polymer comprising crosslinkable groups. In one embodiment, the hydrogel precursor composition comprises between about 1% and about 40% (w/v) of a polymer comprising crosslinkable groups. In one embodiment, the hydrogel precursor composition comprises between about 1% and about 35% (w/v) of a polymer comprising crosslinkable groups. In one embodiment, the hydrogel precursor composition comprises between about 1% and about 30% (w/v) of a polymer comprising crosslinkable groups. In one embodiment, the hydrogel precursor composition comprises between about 1% and about 25% (w/v) of a polymer comprising crosslinkable groups. In one embodiment, the hydrogel precursor composition comprises between about 1% and about 20% (w/v) of a polymer comprising crosslinkable groups. In one embodiment, the hydrogel precursor composition comprises between about 2% and about 20% (w/v) of a polymer comprising crosslinkable groups.

In one embodiment, the hydrogel precursor composition comprises one or more crosslinking agents. In one embodiment, the crosslinking agent comprises one or more photoinitiators. Exemplary photoinitiators include, but are not limited to, benzoin methyl ether; benzoin isopropyl ether; 2,2-diethoxyacetophenone (Irgacure™ 651 photoinitiator); 2,2-dimethoxy-2-phenyl-1-phenylethanone (Esacure™ KB-1 photoinitiator); dimethoxyhydroxyacetophenone; 2-methyl-2-hydroxy propiophenone; 2-naphthalene-sulfonyl chloride; 1-phenyl-1,2-propanedione-2-(O-ethoxy-carbonyl)oxime; 2,4-diethyl thioxanthone; 2-tert-butyl thioxanthone; 2-chlorothioxanthone; 2-propoxy thioxanthone; 2-benzyl-2-dimethylamino-1-(4-morpholinophenyl)butan-1-one (Iracure 369™ photoinitiator); 2-methyl-1-[4-(methylthio)phenyl]-2-morpholino propan-2-one (Iracure907™ photoinitiator); 2-hydroxy-4′-(2-hydroxyethoxy)-2-methylpropiophenone (Irgacure-2959™ photoinitiator); lithium phenyl-2,4,6-trimethylbenzoylphosphinate (LAP); triethanolamine; N-vinyl caprolactam; benzophenone; benzil dimethyl ketal; diethoxyacetophenone; dibutoxyacetophenone; methyl phenyl glycoxylate; 2-ethylthioxanthone; 2-isopropylthioxanthone; phenyl 2-hydroxy-2-propyl ketone; 4-isopropylphenyl 2-hydroxy-2-propyl ketone; 4-n-dodecylphenyl 2-hydroxy-2propyl ketone; 4-(2-hydroxyethoxy)phenyl 2-hydroxy-2propyl ketone; 4-(2-acryloyloxyethoxy)phenyl 2-hydroxy-2-propyl ketone; 1-benzoylcyclohexanol; and Eosin Y.

In one embodiment, the crosslinking agent comprises one or more metal²⁺ ions. In one embodiment, the crosslinking agent comprises one or more metal³⁺ ions. Exemplary metal²⁺ or metal³⁺ ions include, but are not limited to, Fe²⁺, Fe³⁻, Ni²⁺, Zn²⁺, Cu²⁺, Ag²⁺, Au³⁺, Co²⁺, Co³⁺, Cr²⁺, Cr³⁺, Cd²⁺, Mn²⁺, Mg²⁺, Pd²⁺, Pt²⁺, Al³⁺. In one embodiment, the hydrogel precursor composition comprises both one or more photoinitiators and one or more metal^{2-/3+} ions.

In one embodiment, the hydrogel precursor composition comprises between about 1% (w/v) and about 50% (w/v) of one or more crosslinking agents. In one embodiment, the hydrogel precursor composition comprises between about 1% (w/v) and about 45% (w/v) of one or more crosslinking agents. In one embodiment, the hydrogel precursor composition comprises between about 1% (w/v) and about 40% (w/v) of one or more crosslinking agents. In one embodiment, the hydrogel precursor composition comprises between about 1% (w/v) and about 35% (w/v) of one or more crosslinking agents. In one embodiment, the hydrogel precursor composition comprises between about 1% (w/v) and about 30% (w/v) of one or more crosslinking agents. In one embodiment, the hydrogel precursor composition comprises between about 1% (w/v) and about 25% (w/v) of one or more crosslinking agents. In one embodiment, the hydrogel precursor composition comprises between about 1% (w/v) and about 20% (w/v) of one or more crosslinking agents. In one embodiment, the hydrogel precursor composition comprises between about 1% (w/v) and about 15% (w/v) of one or more crosslinking agents. In one embodiment, the hydrogel precursor composition comprises between about 1% (w/v) and about 10% (w/v) of one or more crosslinking agents. In one embodiment, the hydrogel precursor composition comprises between about 3% (w/v) and about 10% (w/v) of one or more crosslinking agents.

In one embodiment, the hydrogel precursor composition comprises one or more solvents. In one embodiment, the solvent is an aqueous solvent. Exemplary aqueous solvents include, but are not limited to, distilled water, deionized water, saline, Dulbecco's phosphate-buffered saline (DPBS), and Ringer's solution. In one embodiment, the solvent comprises DPBS. In one embodiment, the solvent is an organic solvent. Exemplary organic solvents include, but are not limited to, hexanes, benzene, toluene, acetone, diethyl ether, chloroform, dichloromethane, isopropanol, methanol, ethanol, n-propanol, and n-butanol.

In one embodiment, the hydrogel precursor composition comprises one or more therapeutic agents. Exemplary therapeutic agents include, but are not limited to, antimicrobial agents, osteoinductive agents, growth factors, and combinations thereof.

In one embodiment, the hydrogel precursor composition comprises one or more antimicrobial agents. Exemplary antimicrobial agents include, but are not limited to, polymyxin B, vancomycin, cholera toxin, diphtheria toxin, lysostaphin, hemolysin, bacitracin, boceprevir, albavancin, daptomycin, enfuvirtide, oritavancin, teicoplanin, telaprevir, telavancin, guavanin 2, Maximin H5, dermcidin, cecropins, andropin, moricin, ceratotoxin, melittin, magainin, dermaseptin, brevinin-1, esculentins, buforin II, CAP18, LL37, baecin, apidaecins, prophenin, indolicidin, AMP Tet213, chlorhexidine, a chlorhexadine salt, triclosan, polymyxin, tetracycline, an amino glycoside (e.g., gentamicin or Tobramycin™), rifampicin, erythromycin, neomycin, chloramphenicol, miconazole, a quinolone, penicillin, fusidic acid, cephalosporin, mupirocin, metronidazole, secropin, protegrin, bacteriolcin, defensin, nitrofurazone, mafenide, aracyclovir, clindamycin, lincomycin, sulfonamide, norfloxacin, pefloxacin, nalidizic acid, cinnamycin, anti-DEFA5, duramycin, nisin, pediocin, Abaecin, Ct-AMP1, Apidaecin IA, Apidaecin IB, Bactenecin, BACTENECIN 5, BACTENECIN 7, Bactericidin B-2, Aurein family, SMAP-29, Temporin B, Pleurocidin, Tachyplesin III, LL-37, Citropin 1.1, BMAP-27, BMAP-28, Agelaia-MP, Temporin 1O1a, NA-CATH, Histatins, Latarcin, Halocidin, Bombinin, Cathelicidin, Malacidin, MP196, MS100a7a15, Murepavadin, Myticin, Mytilin, Paenibacterin, Pardaxin, Peptaibol, SAAP-148, Sarcotoxin, Stomoxyn, Tachyplesin, thioester-containing protein 1, Thionin, Alamethicin, Arenicin, dermorphins, deltorphins, dermaseptins, pseudin, bombesins, maculatins, LEAP2, Efrapeptin, Arylomycins, Capreomycin, Gramicidin B, Antiamoebin, Bacillomycin, Teixobactin, Tyrothricin, Viomycin, and oxalic acid. In one embodiment, the antimicrobial agent is an antimicrobial peptide (AMP). In one embodiment, the antimicrobial peptide is AMP Tet213.

In one embodiment, the hydrogel precursor composition comprises between about 0% and about 30% (w/v) antimicrobial agent. In one embodiment, the hydrogel precursor composition comprises between about 0% and about 25% (w/v) antimicrobial agent. In one embodiment, the hydrogel precursor composition comprises between about 0% and about 20% (w/v) antimicrobial agent. In one embodiment, the hydrogel precursor composition comprises between about 0% and about 15% (w/v) antimicrobial agent. In one embodiment, the hydrogel precursor composition comprises between about 0% and about 10% (w/v) antimicrobial agent. In one embodiment, the hydrogel precursor composition comprises between about 0% and about 5% (w/v) antimicrobial agent. In one embodiment, the hydrogel precursor composition comprises between about 0% and about 2% (w/v) antimicrobial agent.

In one embodiment, the hydrogel precursor composition comprises one or more osteoinductive agents. In one embodiment, the osteoinductive agent comprises silicate nanoparticles. The silicate nanoparticles can be any silicate nanoparticles known to a person of skill in the art. In one embodiment, the silicate nanoparticles comprise one or more metals. Exemplary metals include, but are not limited to, calcium, aluminum, silver, gold, platinum, palladium, lithium, magnesium, sodium, titanium, vanadium, chromium, manganese, iron, cobalt, nickel, copper, zinc, and iridium. In one embodiment, the silicate nanoparticles are laponite nanoparticles. In one embodiment, the osteoinductive agent comprises a calcium salt. Exemplary calcium salts include, but are not limited to, calcium phosphate, calcium sulfate, calcium hydroxide, calcium bromide, calcium fluoride, calcium iodide, and calcium hydride. In one embodiment, the osteoinductive agent comprises bioglass. In one embodiment, the osteoinductive agent comprises hydroxyapatite. In one embodiment, the osteoinductive agent comprises demineralized bone matrix (DBM). In one embodiment, the osteoinductive agent comprises a combination of osteoinductive agents. In one embodiment, the osteoinductive agent comprises a mixture of calcium phosphate, calcium sulfate, and bioglass.

In one embodiment, the hydrogel precursor composition comprises between about 0% and about 50% (w/v) of an osteoinductive agent. In one embodiment, the hydrogel precursor composition comprises between about 0% and about 45% (w/v) of an osteoinductive agent. In one embodiment, the hydrogel precursor composition comprises between about 0% and about 40% (w/v) of an osteoinductive agent. In one embodiment, the hydrogel precursor composition comprises between about 0% and about 35% (w/v) of an osteoinductive agent. In one embodiment, the hydrogel precursor composition comprises between about 0% and about 30% (w/v) of an osteoinductive agent. In one embodiment, the hydrogel precursor composition comprises between about 0% and about 25% (w/v) silicate nanoparticles. In one embodiment, the hydrogel precursor composition comprises between about 0% and about 20% (w/v) of an osteoinductive agent. In one embodiment, the hydrogel precursor composition comprises between about 0% and about 15% (w/v) of an osteoinductive agent. In one embodiment, the hydrogel precursor composition comprises between about 0% and about 10% (w/v) of an osteoinductive agent. In one embodiment, the hydrogel precursor composition comprises between about 0% and about 2% (w/v) of an osteoinductive agent.

In one embodiment, the hydrogel precursor composition comprises one or more growth factors. The growth factor can be any growth factor known to a person of skill in the art. Exemplary growth factors include, but are not limited to, Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF) Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Foetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Neurotrophins, Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF). In one embodiment, the growth factor comprises VEGF.

In one embodiment, the hydrogel precursor composition comprises between about 0.001 mg/mL and about 1000 mg/mL growth factors. In one embodiment, the hydrogel precursor composition comprises between about 0.001 μg/mL and about 900 μg/mL growth factors. In one embodiment, the hydrogel precursor composition comprises between about 0.001 μg/mL and about 800 μg/mL growth factors. In one embodiment, the hydrogel precursor composition comprises between about 0.001 μg/mL and about 700 μg/mL growth factors. In one embodiment, the hydrogel precursor composition comprises between about 0.001 mg/mL and about 600 μg/mL growth factors. In one embodiment, the hydrogel precursor composition comprises between about 0.001 μg/mL and about 500 μg/mL growth factors. In one embodiment, the hydrogel precursor composition comprises between about 0.001 μg/mL and about 400 μg/mL growth factors. In one embodiment, the hydrogel precursor composition comprises between about 0.001 μg/mL and about 300 μg/mL growth factors. In one embodiment, the hydrogel precursor composition comprises between about 0.001 μg/mL and about 200 μg/mL growth factors. In one embodiment, the hydrogel precursor composition comprises between about 0.001 μg/mL and about 120 μg/mL growth factors. In one embodiment, the hydrogel precursor composition comprises between about 0.01 μg/mL and about 120 μg/mL growth factors. In one embodiment, the hydrogel precursor composition comprises between about 0.05 μg/mL and about 120 μg/mL growth factors. In one embodiment, the hydrogel precursor composition comprises between about 0.08 μg/mL and about 120 mg/mL growth factors.

### Hydrogels

In one aspect, the invention relates to a hydrogel. In one embodiment, the hydrogel comprises a crosslinked polymer. In one embodiment, the hydrogel comprises a crosslinked natural polymer. Exemplary natural polymers are described elsewhere herein. In one embodiment, the hydrogel comprises a crosslinked synthetic polymer. In one embodiment, the hydrogel comprises crosslinked gelatin. In one embodiment, the crosslinked polymer comprises segments that are crosslinked. Crosslinked segments comprise those formed from crosslinking reactions of functional groups including, but not limited to, methyl acrylate, ethyl acrylate, propyl acrylate, methyl methacrylate, ethyl methacrylate, methacryloyl, catechol, ethylene oxide, and propylene oxide. In one embodiment, the crosslinked segments comprise those formed from crosslinking reactions between methacryloyl groups. In one embodiment, the crosslinked segments comprise those formed from crosslinking reactions between catechol groups. In one embodiment, the crosslinked segments comprise those formed from crosslinking reactions between both catechol groups and methacryloyl groups.

In one embodiment, the hydrogel comprises one or more solvents. In one embodiment, the solvent is an aqueous solvent. Exemplary aqueous solvents are described elsewhere herein. In one embodiment, the solvent is an organic solvent. Exemplary organic solvents are described elsewhere herein.

In one embodiment, the hydrogel comprises one or more crosslinking agents. In one embodiment, the crosslinking agent comprises one or more photoinitiators. Exemplary photoinitiators are described elsewhere herein. In one embodiment, the crosslinking agent comprises one or more metal²⁺ ions. In one embodiment, the crosslinking agent comprises one or more metal³⁺ ions. Exemplary metal²⁺ and metal³⁺ ions are described elsewhere herein. In one embodiment, the hydrogel comprises both one or more photoinitiators and one or more metal^{2+/3+} ions.

In one embodiment, the hydrogel comprises one or more therapeutic agents. Exemplary therapeutic agents include, but are not limited to, antimicrobial agents, osteoinductive agents, growth factors, and combinations thereof.

In one embodiment, the hydrogel comprises one or more antimicrobial agents. Exemplary antimicrobial agents are described elsewhere herein. In one embodiment, the antimicrobial agent is an antimicrobial peptide (AMP). In one embodiment, the antimicrobial peptide is AMP Tet213.

In one embodiment, the hydrogel comprises between about 0% and about 30% (w/v) antimicrobial agent. In one embodiment, the hydrogel comprises between about 0% and about 25% (w/v) antimicrobial agent. In one embodiment, the hydrogel comprises between about 0% and about 20% (w/v) antimicrobial agent. In one embodiment, the hydrogel comprises between about 0% and about 15% (w/v) antimicrobial agent. In one embodiment, the hydrogel comprises between about 0% and about 10% (w/v) antimicrobial agent. In one embodiment, the hydrogel comprises between about 0% and about 5% (w/v) antimicrobial agent. In one embodiment, the hydrogel comprises between about 0% and about 2% (w/v) antimicrobial agent.

In one embodiment, the hydrogel comprises one or more osteoinductive agents. Exemplary osteoinductive agents are described elsewhere herein. In one embodiment, the osteoinductive agent comprises silicate nanoparticles. Exemplary silicate nanoparticles are described elsewhere herein. In one embodiment, the silicate nanoparticles are laponite nanoparticles.

In one embodiment, the hydrogel comprises between about 0% and about 50% (w/v) of an osteoinductive agent. In one embodiment, the hydrogel comprises between about 0% and about 45% (w/v) of an osteoinductive agent. In one embodiment, the hydrogel comprises between about 0% and about 40% (w/v) of an osteoinductive agent. In one embodiment, the hydrogel comprises between about 0% and about 35% (w/v) of an osteoinductive agent. In one embodiment, the hydrogel comprises between about 0% and about 30% (w/v) of an osteoinductive agent. In one embodiment, the hydrogel comprises between about 0% and about 25% (w/v) of an osteoinductive agent. In one embodiment, the hydrogel comprises between about 0% and about 20% (w/v) of an osteoinductive agent. In one embodiment, the hydrogel comprises between about 0% and about 15% (w/v) of an osteoinductive agent. In one embodiment, the hydrogel comprises between about 0% and about 10% (w/v) of an osteoinductive agent.

In one embodiment, the hydrogel comprises one or more growth factors. Exemplary growth factors are described elsewhere herein.

In one embodiment, the hydrogel comprises gelatin methacryloyl, one or more photoinitiators, and does not comprise an antimicrobial agent or silicate nanoparticles (i.e. GelMA). In one embodiment, the GelMA hydrogel comprises an elastic modulus of between about 1 kPa and about 1500 kPa. In one embodiment, the GelMA hydrogel comprises an elastic modulus of between about 1 kPa and about 1300 kPa. In one embodiment, the GelMA hydrogel comprises an elastic modulus of between about 1 kPa and about 1100 kPa. In one embodiment, the GelMA hydrogel comprises an elastic modulus of between about 1 kPa and about 900 kPa. In one embodiment, the GelMA hydrogel comprises an elastic modulus of between about 1 kPa and about 700 kPa. In one embodiment, the GelMA hydrogel comprises an elastic modulus of between about 1 kPa and about 500 kPa. In one embodiment, the GelMA hydrogel comprises an elastic modulus of between about 1 kPa and about 300 kPa. In one embodiment, the GelMA hydrogel comprises an elastic modulus of between about 1 kPa and about 175 kPa.

In one embodiment, the GelMA hydrogel comprises a compressive modulus between about 1 kPa and about 150 kPa. In one embodiment, the GelMA hydrogel comprises a compressive modulus between about 1 kPa and about 140 kPa. In one embodiment, the GelMA hydrogel comprises a compressive modulus between about 1 kPa and about 130 kPa. In one embodiment, the GelMA hydrogel comprises a compressive modulus between about 1 kPa and about 120 kPa. In one embodiment, the GelMA hydrogel comprises a compressive modulus between about 1 kPa and about 110 kPa. In one embodiment, the GelMA hydrogel comprises a compressive modulus between about 1 kPa and about 100 kPa. In one embodiment, the GelMA hydrogel comprises a compressive modulus between about 1 kPa and about 90 kPa. In one embodiment, the GelMA hydrogel comprises a compressive modulus between about 1 kPa and about 80 kPa. In one embodiment, the GelMA hydrogel comprises a compressive modulus between about 1 kPa and about 70 kPa. In one embodiment, the GelMA hydrogel comprises a compressive modulus between about 1 kPa and about 60 kPa. In one embodiment, the GelMA hydrogel comprises a compressive modulus between about 5 kPa and about 60 kPa.

In one embodiment, the GelMA hydrogel comprises an ultimate stress of between about 1 kPa and about 150 kPa. In one embodiment, the GelMA hydrogel comprises an ultimate stress of between about 1 kPa and about 140 kPa. In one embodiment, the GelMA hydrogel comprises an ultimate stress of between about 1 kPa and about 130 kPa. In one embodiment, the GelMA hydrogel comprises an ultimate stress of between about 1 kPa and about 120 kPa. In one embodiment, the GelMA hydrogel comprises an ultimate stress of between about 1 kPa and about 110 kPa. In one embodiment, the GelMA hydrogel comprises an ultimate stress of between about 1 kPa and about 100 kPa. In one embodiment, the GelMA hydrogel comprises an ultimate stress of between about 1 kPa and about 90 kPa. In one embodiment, the GelMA hydrogel comprises an ultimate stress of between about 1 kPa and about 80 kPa. In one embodiment, the GelMA hydrogel comprises an ultimate stress of between about 1 kPa and about 70 kPa. In one embodiment, the GelMA hydrogel comprises an ultimate stress of between about 1 kPa and about 60 kPa. In one embodiment, the GelMA hydrogel comprises an ultimate stress of between about 1 kPa and about 50 kPa. In one embodiment, the GelMA hydrogel comprises an ultimate stress of between about 1 kPa and about 40 kPa. In one embodiment, the GelMA hydrogel comprises an ultimate stress of between about 1 kPa and about 30 kPa.

In one embodiment, the GelMA hydrogel comprises an extensibility of between about 1% to about 150%. In one embodiment, the GelMA hydrogel comprises an extensibility of between about 1% to about 140%. In one embodiment, the GelMA hydrogel comprises an extensibility of between about 1% to about 130%. In one embodiment, the GelMA hydrogel comprises an extensibility of between about 1% to about 120%. In one embodiment, the GelMA hydrogel comprises an extensibility of between about 1% to about 110%. In one embodiment, the GelMA hydrogel comprises an extensibility of between about 1% to about 100%. In one embodiment, the GelMA hydrogel comprises an extensibility of between about 1% to about 90%. In one embodiment, the GelMA hydrogel comprises an extensibility of between about 1% to about 80%. In one embodiment, the GelMA hydrogel comprises an extensibility of between about 1% to about 70%. In one embodiment, the GelMA hydrogel comprises an extensibility of between about 1% to about 60%. In one embodiment, the GelMA hydrogel comprises an extensibility of between about 1% to about 50%. In one embodiment, the GelMA hydrogel comprises an extensibility of between about 1% to about 40%. In one embodiment, the GelMA hydrogel comprises an extensibility of between about 10% to about 40%.

In one embodiment, the hydrogel comprises gelatin methacryloyl modified with catchecol groups, one or more photoinitiators, one or more metal^{2+/3+} ions, and does not comprise an antimicrobial agent of silicate nanoparticles (i.e. GelMAC). In one embodiment, the GelMAC hydrogel comprises an elastic modulus of between about 1 kPa and about 300 kPa. In one embodiment, the GelMAC hydrogel comprises an elastic modulus of between about 1 kPa and about 280 kPa. In one embodiment, the GelMAC hydrogel comprises an elastic modulus of between about 1 kPa and about 260 kPa. In one embodiment, the GelMAC hydrogel comprises an elastic modulus of between about 1 kPa and about 240 kPa. In one embodiment, the GelMAC hydrogel comprises an elastic modulus of between about 1 kPa and about 220 kPa. In one embodiment, the GelMAC hydrogel comprises an elastic modulus of between about 1 kPa and about 200 kPa. In one embodiment, the GelMAC hydrogel comprises an elastic modulus of between about 1 kPa and about 180 kPa. In one embodiment, the GelMAC hydrogel comprises an elastic modulus of between about 1 kPa and about 170 kPa. In one embodiment, the GelMAC hydrogel comprises an elastic modulus of between about 1 kPa and about 160 kPa. In one embodiment, the GelMAC hydrogel comprises an elastic modulus of between about 1 kPa and about 150 kPa. In one embodiment, the GelMAC hydrogel comprises an elastic modulus of between about 1 kPa and about 140 kPa. In one embodiment, the GelMAC hydrogel comprises an elastic modulus of between about 1 kPa and about 130 kPa. In one embodiment, the elastic modulus of the GelMAC hydrogel is dependent on the concentration of metal coordinated to the catechol groups.

In one embodiment, the GelMAC hydrogel comprises an ultimate stress of between about 1 kPa and about 150 kPa. In one embodiment, the GelMAC hydrogel comprises an ultimate stress of between about 1 kPa and about 140 kPa. In one embodiment, the GelMAC hydrogel comprises an ultimate stress of between about 1 kPa and about 130 kPa. In one embodiment, the GelMAC hydrogel comprises an ultimate stress of between about 1 kPa and about 120 kPa. In one embodiment, the GelMAC hydrogel comprises an ultimate stress of between about 1 kPa and about 110 kPa. In one embodiment, the GelMAC hydrogel comprises an ultimate stress of between about 1 kPa and about 100 kPa. In one embodiment, the GelMAC hydrogel comprises an ultimate stress of between about 1 kPa and about 90 kPa. In one embodiment, the GelMAC hydrogel comprises an ultimate stress of between about 1 kPa and about 80 kPa. In one embodiment, the GelMAC hydrogel comprises an ultimate stress of between about 1 kPa and about 70 kPa. In one embodiment, the GelMAC hydrogel comprises an ultimate stress of between about 10 kPa and about 70 kPa. In one embodiment, the ultimate stress of the GelMAC hydrogel is dependent on the concentration of metal coordinated to the catechol groups.

In one embodiment, the GelMAC hydrogel comprises an extensibility of between about 1% to about 200%. In one embodiment, the GelMAC hydrogel comprises an extensibility of between about 1% to about 190%. In one embodiment, the GelMAC hydrogel comprises an extensibility of between about 1% to about 200%. In one embodiment, the GelMAC hydrogel comprises an extensibility of between about 1% to about 180%. In one embodiment, the GelMAC hydrogel comprises an extensibility of between about 1% to about 160%. In one embodiment, the GelMAC hydrogel comprises an extensibility of between about 1% to about 140%. In one embodiment, the GelMAC hydrogel comprises an extensibility of between about 1% to about 125%. In one embodiment, the GelMAC hydrogel comprises an extensibility of between about 10% to about 125%. In one embodiment, the GelMAC hydrogel comprises an extensibility of between about 20% to about 125%. In one embodiment, the GelMAC hydrogel comprises an extensibility of between about 30% to about 125%. In one embodiment, the GelMAC hydrogel comprises an extensibility of between about 40% to about 125%. In one embodiment, the GelMAC hydrogel comprises an extensibility of between about 50% to about 125%. In one embodiment, the extensibility of the GelMAC hydrogel is dependent on the concentration of metal coordinated to the catechol groups.

In one embodiment, the hydrogel comprises gelatin methacryloyl, one or more photoinitiators, an AMP antimicrobial agent, and does not comprise silicate nanoparticles (i.e. GelAMP). In one embodiment, the GelAMP hydrogel comprises an elastic modulus of between about 1 kPa and about 150 kPa. In one embodiment, the GelAMP hydrogel comprises an elastic modulus of between about 1 kPa and about 140 kPa. In one embodiment, the GelAMP hydrogel comprises an elastic modulus of between about 1 kPa and about 130 kPa. In one embodiment, the GelAMP hydrogel comprises an elastic modulus of between about 1 kPa and about 120 kPa. In one embodiment, the GelAMP hydrogel comprises an elastic modulus of between about 1 kPa and about 110 kPa. In one embodiment, the GelAMP hydrogel comprises an elastic modulus of between about 1 kPa and about 100 kPa. In one embodiment, the GelAMP hydrogel comprises an elastic modulus of between about 1 kPa and about 90 kPa. In one embodiment, the GelAMP hydrogel comprises an elastic modulus of between about 1 kPa and about 80 kPa. In one embodiment, the GelAMP hydrogel comprises an elastic modulus of between about 10 kPa and about 80 kPa. In one embodiment, the GelAMP hydrogel comprises an elastic modulus of between about 20 kPa and about 80 kPa. In one embodiment, the GelAMP hydrogel comprises an elastic modulus of between about 30 kPa and about 80 kPa. In one embodiment, the GelAMP hydrogel comprises an elastic modulus of between about 40 kPa and about 80 kPa.

In one embodiment, the GelAMP hydrogel comprises a compressive modulus of between about 1 kPa to about 150 kPa. In one embodiment, the GelAMP hydrogel comprises a compressive modulus of between about 1 kPa to about 130 kPa. In one embodiment, the GelAMP hydrogel comprises a compressive modulus of between about 1 kPa to about 110 kPa. In one embodiment, the GelAMP hydrogel comprises a compressive modulus of between about 1 kPa to about 90 kPa. In one embodiment, the GelAMP hydrogel comprises a compressive modulus of between about 1 kPa to about 80 kPa. In one embodiment, the GelAMP hydrogel comprises a compressive modulus of between about 1 kPa to about 70 kPa. In one embodiment, the GelAMP hydrogel comprises a compressive modulus of between about 10 kPa to about 70 kPa. In one embodiment, the GelAMP hydrogel comprises a compressive modulus of between about 20 kPa to about 70 kPa. In one embodiment, the GelAMP hydrogel comprises a compressive modulus of between about 30 kPa to about 70 kPa. In one embodiment, the GelAMP hydrogel comprises a compressive modulus of between about 40 kPa to about 70 kPa.

In one embodiment, the GelAMP hydrogel comprises an ultimate stress of between about 1 kPa to about 120 kPa. In one embodiment, the GelAMP hydrogel comprises an ultimate stress of between about 1 kPa to about 110 kPa. In one embodiment, the GelAMP hydrogel comprises an ultimate stress of between about 1 kPa to about 100 kPa. In one embodiment, the GelAMP hydrogel comprises an ultimate stress of between about 1 kPa to about 90 kPa. In one embodiment, the GelAMP hydrogel comprises an ultimate stress of between about 1 kPa to about 80 kPa. In one embodiment, the GelAMP hydrogel comprises an ultimate stress of between about 1 kPa to about 70 kPa. In one embodiment, the GelAMP hydrogel comprises an ultimate stress of between about 1 kPa to about 60 kPa. In one embodiment, the GelAMP hydrogel comprises an ultimate stress of between about 1 kPa to about 50 kPa. In one embodiment, the GelAMP hydrogel comprises an ultimate stress of between about 1 kPa to about 40 kPa. In one embodiment, the GelAMP hydrogel comprises an ultimate stress of between about 1 kPa to about 30 kPa. In one embodiment, the GelAMP hydrogel comprises an ultimate stress of between about 10 kPa to about 30 kPa.

In one embodiment, the GelAMP hydrogel comprises an extensibility of between about 1% to about 120%. In one embodiment, the GelAMP hydrogel comprises an extensibility of between about 1% to about 110%. In one embodiment, the GelAMP hydrogel comprises an extensibility of between about 1% to about 100%. In one embodiment, the GelAMP hydrogel comprises an extensibility of between about 1% to about 90%. In one embodiment, the GelAMP hydrogel comprises an extensibility of between about 1% to about 80%. In one embodiment, the GelAMP hydrogel comprises an extensibility of between about 1% to about 70%. In one embodiment, the GelAMP hydrogel comprises an extensibility of between about 1% to about 60%. In one embodiment, the GelAMP hydrogel comprises an extensibility of between about 1% to about 50%. In one embodiment, the GelAMP hydrogel comprises an extensibility of between about 1% to about 40%. In one embodiment, the GelAMP hydrogel comprises an extensibility of between about 10% to about 40%.

In one embodiment, the hydrogel comprises gelatin methacryloyl, one or more photoinitiators, silicate nanoparticles, and does not comprise an antimicrobial agent (i.e. GelMa w/ SN). In one embodiment, the GelMA w/ SN hydrogel comprises an elastic modulus of between about 1 kPa to about 300 kPa. In one embodiment, the GelMA w/ SN hydrogel comprises an elastic modulus of between about 1 kPa to about 280 kPa. In one embodiment, the GelMA w/ SN hydrogel comprises an elastic modulus of between about 1 kPa to about 260 kPa. In one embodiment, the GelMA w/ SN hydrogel comprises an elastic modulus of between about 1 kPa to about 240 kPa. In one embodiment, the GelMA w/ SN hydrogel comprises an elastic modulus of between about 1 kPa to about 220 kPa. In one embodiment, the GelMA w/ SN hydrogel comprises an elastic modulus of between about 1 kPa to about 200 kPa. In one embodiment, the GelMA w/ SN hydrogel comprises an elastic modulus of between about 1 kPa to about 180 kPa. In one embodiment, the GelMA w/ SN hydrogel comprises an elastic modulus of between about 1 kPa to about 160 kPa. In one embodiment, the GelMA w/ SN hydrogel comprises an elastic modulus of between about 20 kPa to about 160 kPa. In one embodiment, the GelMA w/ SN hydrogel comprises an elastic modulus of between about 40 kPa and about 160 kPa. In one embodiment, the GelMA w/ SN hydrogel comprises an elastic modulus of between about 60 kPa to about 160 kPa. In one embodiment, the GelMA w/ SN hydrogel comprises an elastic modulus of between about 70 kPa to about 160 kPa. In one embodiment, the elastic modulus of the GelMA w/ SN hydrogel is dependent on the concentration of silicate nanoparticles in the hydrogel.

In one embodiment, the compressive modulus of the GelMA w/ SN hydrogel comprises between about 1 kPa to about 300 kPa. In one embodiment, the compressive modulus of the GelMA w/ SN hydrogel comprises between about 1 kPa to about 280 kPa. In one embodiment, the compressive modulus of the GelMA w/ SN hydrogel comprises between about 1 kPa to about 260 kPa. In one embodiment, the compressive modulus of the GelMA w/ SN hydrogel comprises between about 1 kPa to about 240 kPa. In one embodiment, the compressive modulus of the GelMA w/ SN hydrogel comprises between about 1 kPa to about 220 kPa. In one embodiment, the compressive modulus of the GelMA w/ SN hydrogel comprises between about 1 kPa to about 200 kPa. In one embodiment, the compressive modulus of the GelMA w/ SN hydrogel comprises between about 1 kPa to about 180 kPa. In one embodiment, the compressive modulus of the GelMA w/ SN hydrogel comprises between about 1 kPa to about 160 kPa. In one embodiment, the compressive modulus of the GelMA w/ SN hydrogel comprises between about 1 kPa to about 140 kPa. In one embodiment, the compressive modulus of the GelMA w/ SN hydrogel comprises between about 1 kPa to about 120 kPa. In one embodiment, the compressive modulus of the GelMA w/ SN hydrogel comprises between about 1 kPa to about 100 kPa. In one embodiment, the compressive modulus of the GelMA w/ SN hydrogel comprises between about 20 kPa to about 100 kPa. In one embodiment, the compressive modulus of the GelMA w/ SN hydrogel is dependent on the concentration of silicate nanoparticles in the hydrogel.

### Method of Making a Hydrogel

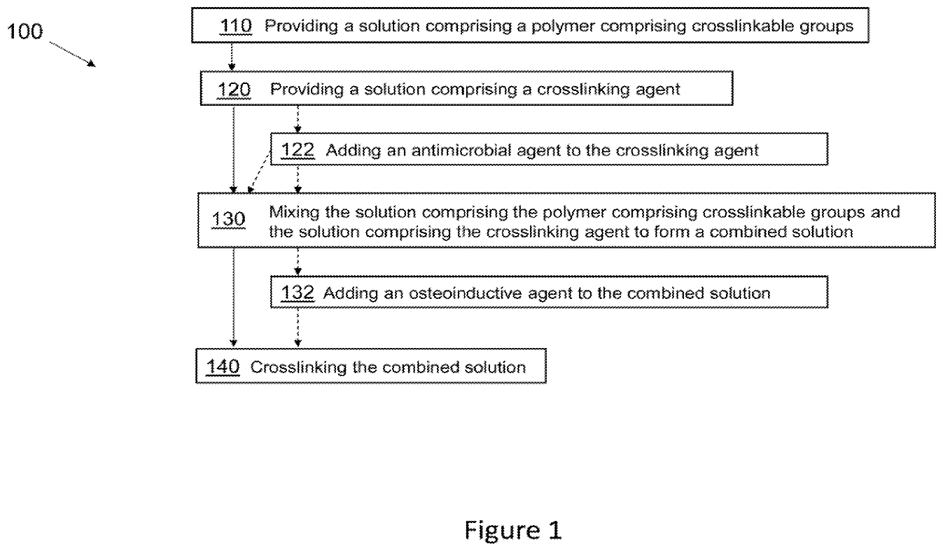

In another aspect, the invention relates to a method of producing a hydrogel. Exemplary process 100 is shown in FIG. 1. In step 110, a solution comprising a polymer comprising crosslinkable groups is provided. In step 120, a solution comprising a crosslinking agent is provided. In step 130, the solution comprising a polymer comprising crosslinkable groups is mixed with the solution comprising a crosslinking agent to form a combined solution. In step 140, the combined solution is crosslinked.

In step 110, the polymer may comprise any crosslinkable groups known to a person of skill in the art. Exemplary crosslinkable are described elsewhere herein. In one embodiment, the crosslinkable groups comprise methacryloyl groups. In one embodiment, the crosslinkable groups comprise catechol groups. In one embodiment, the crosslinkable groups comprise both methacryloyl groups and catechol groups. In one embodiment, the polymer comprises gelatin. In one embodiment, the solution of polymer comprising crosslinkable groups comprises an aqueous solvent. Exemplary aqueous solvents are described elsewhere herein. In one embodiment, the solution of polymer comprising crosslinkable groups comprises an organic solvent. Exemplary organic solvents are described elsewhere herein.

In step 120, the crosslinking agent can be any crosslinking agent known to those of skill in the art. In one embodiment, the crosslinking agent comprises one or more photoinitiators. Exemplary photoinitiators are described elsewhere herein. In one embodiment, the crosslinking agent comprises one or more metal²⁺ ions. Exemplary metal²⁺ ions are described elsewhere herein. In one embodiment, the crosslinking agent comprises one or more metal³⁺ ions. Exemplary metal³⁺ ions are described elsewhere herein. In one embodiment, the crosslinking agent comprises both a photoinitiator and a metal^{2+/3+} ion. In one embodiment, the solution of the crosslinking agent comprises an aqueous solvent. Exemplary aqueous solvents are described elsewhere herein. In one embodiment, the solution of the crosslinking agent comprises an organic solvent. Exemplary organic solvents are described elsewhere herein.

In one embodiment, a compound comprising crosslinkable groups is reacted with the polymer to form a polymer comprising crosslinkable groups. In one embodiment, the polymer comprises gelatin. The compound comprising crosslinkable groups can be any compound known to those of skill in the art. In one embodiment, the compound comprising crosslinkable groups is an anhydride. In one embodiment, the compound comprising crosslinkable groups is an acid halide. In one embodiment, the compound comprising crosslinkable groups is a carboxylic acid. In one embodiment, the compound comprising crosslinkable groups is a diol. In one embodiment, the compound comprising crosslinkable groups is acrylic anhydride. In one embodiment, the compound comprising crosslinkable groups is methacrylic anhydride. In one embodiment, the compound comprising crosslinkable groups is acryloyl chloride. In one embodiment, the compound comprising crosslinkable groups is acryloyl bromide. In one embodiment, the compound comprising crosslinkable groups is methacryloyl chloride. In one embodiment, the compound comprising crosslinkable groups is methacryloyl bromide. In one embodiment, the compound comprising crosslinkable groups is acrylic acid. In one embodiment, the compound comprising crosslinkable groups is glycidyl methacrylate. In one embodiment, the compound comprising crosslinkable groups is methacrylic acid. In one embodiment, the compound comprising crosslinkable groups is dopamine.

In one embodiment, the polymer is dissolved or dispersed in a solvent. Exemplary organic and aqueous solvents are described elsewhere herein. In one embodiment, the polymer dissolved or dispersed in a solvent forms a solution.

In one embodiment, the solution of polymer is cooled before it is reacted with a compound comprising crosslinkable groups. In one embodiment, the solution of polymer is cooled to a temperature of between about 0° C. and about 20° C.

In one embodiment, the solution of polymer is heated before it is reacted with a compound comprising crosslinkable groups. In one embodiment, the solution of polymer is heated between 30° C. and 150° C. In one embodiment, the solution of polymer is heated between 30° C. and 140° C. In one embodiment, the solution of polymer is heated between 30° C. and 130° C. In one embodiment, the solution of polymer is heated between 30° C. and 120° C. In one embodiment, the solution of polymer is heated between 30° C. and 110° C. In one embodiment, the solution of polymer is heated between 30° C. and 100° C. In one embodiment, the solution of polymer is heated between 30° C. and 90° C. In one embodiment, the solution of polymer is heated between 30° C. and 80° C. In one embodiment, the solution of polymer is heated between 30° C. and 70° C. In one embodiment, the solution of polymer is heated between 40° C. and 70° C. In one embodiment, the solution of polymer is heated between 50° C. and 70° C. In one embodiment, the solution of polymer is heated between 55° C. and 65° C.

In one embodiment, the solution of polymer is heated for 10 minutes to 24 hours before it is reacted with a compound comprising crosslinkable groups. In one embodiment, the solution of polymer is heated for 10 minutes to 20 hours before it is reacted with a compound comprising crosslinkable groups. In one embodiment, the solution of polymer is heated for 10 minutes to 15 hours before it is reacted with a compound comprising crosslinkable groups. In one embodiment, the solution of polymer is heated for 10 minutes to 10 hours before it is reacted with a compound comprising crosslinkable groups. In one embodiment, the solution of polymer is heated for 10 minutes to 8 hours before it is reacted with a compound comprising crosslinkable groups. In one embodiment, the solution of polymer is heated for 10 minutes to 6 hours before it is reacted with a compound comprising crosslinkable groups. In one embodiment, the solution of polymer is heated for 10 minutes to 4 hours before it is reacted with a compound comprising crosslinkable groups. In one embodiment, the solution of polymer is heated for 10 minutes to 2 hours before it is reacted with a compound comprising crosslinkable groups. In one embodiment, the solution of polymer is heated for 10 minutes to 1.5 hours before it is reacted with a compound comprising crosslinkable groups. In one embodiment, the solution of polymer is heated for 30 minutes to 1.5 hours before it is reacted with a compound comprising crosslinkable groups. In one embodiment, heating the polymer aids in the dissolution of the polymer in a solvent to form a solution.

In one embodiment, the heating of the polymer solution is continued as the compound comprising crosslinkable groups is added. The polymer solution can be heated at any temperature described elsewhere herein. In one embodiment, the polymer solution is stirred during the addition of the compound comprising crosslinkable groups. In one embodiment, the compound comprising crosslinkable groups is added to the solution of polymer. In one embodiment, a solution of the compound comprising crosslinkable groups is added to the solution of polymer. In one embodiment, the solution of the compound comprising crosslinkable groups comprises an aqueous solvent. Exemplary aqueous solvents are described elsewhere herein. In one embodiment, the solution of the compound comprising crosslinkable groups comprises an organic solvent. Exemplary organic solvents are described elsewhere herein. In one embodiment, the solution of the compound comprising crosslinkable groups is added dropwise to the polymer solution. In one embodiment, the compound comprising crosslinkable groups is added dropwise between 1 minute and 18 hours. In one embodiment, the compound comprising crosslinkable groups is added dropwise between 1 minute and 17 hours. In one embodiment, the compound comprising crosslinkable groups is added dropwise between 1 minute and 16 hours. In one embodiment, the compound comprising crosslinkable groups is added dropwise between 1 minute and 15 hours. In one embodiment, the compound comprising crosslinkable groups is added dropwise between 1 minute and 14 hours. In one embodiment, the compound comprising crosslinkable groups is added dropwise between 1 minute and 13 hours. In one embodiment, the compound comprising crosslinkable groups is added dropwise between 1 minute and 12 hours. In one embodiment, the compound comprising crosslinkable groups is added dropwise between 1 minute and 11 hours. In one embodiment, the compound comprising crosslinkable groups is added dropwise between 1 minute and 10 hours. In one embodiment, the compound comprising crosslinkable groups is added dropwise between 1 minute and 9 hours. In one embodiment, the compound comprising crosslinkable groups is added dropwise between 1 minute and 8 hours. In one embodiment, the compound comprising crosslinkable groups is added dropwise between 1 minute and 7 hours. In one embodiment, the compound comprising crosslinkable groups is added dropwise between 1 minute and 6 hours. In one embodiment, the compound comprising crosslinkable groups is added dropwise between 1 minute and 5 hours. In one embodiment, the compound comprising crosslinkable groups is added dropwise between 1 minute and 4 hours.

In one embodiment, the mixture of polymer and a compound comprising crosslinkable groups comprises between about about 5% (w/v) to about 95% (w/v) polymer. In one embodiment, the mixture of polymer and a compound comprising crosslinkable groups comprises between about 0.001% (w/v) to about 95% (w/v) of a compound comprising crosslinkable groups. In one embodiment, the mixture comprises between about 0.001% (w/v) to about 85% (w/v) of a compound comprising crosslinkable groups. In one embodiment, the mixture comprises between about 0.001% (w/v) to about 75% (w/v) of a compound comprising crosslinkable groups. In one embodiment, the mixture comprises between about 0.001% (w/v) to about 65% (w/v) of a compound comprising crosslinkable groups. In one embodiment, the mixture comprises between about 0.001% (w/v) to about 55% (w/v) of a compound comprising crosslinkable groups. In one embodiment, the mixture comprises between about 0.001% (w/v) to about 45% (w/v) of a compound comprising crosslinkable groups. In one embodiment, the mixture comprises between about 0.008% (w/v) to about 45% (w/v) of a compound comprising crosslinkable groups. In one embodiment, the mixture of polymer and a compound comprising one or more crosslinkable groups reacts to form polymer modified with crosslinkable groups. In one embedment, the polymer comprises gelatin and reacts with a compound comprising one or more crosslinkable groups to form gelatin modified with crosslinkable groups

In one embodiment, the polymer modified with crosslinkable groups is dialyzed to remove any unreacted compound comprising crosslinkable groups. In one embodiment, the dialysis buffer comprises an aqueous solvent. Exemplary aqueous solvents are described elsewhere herein. In one embodiment, the dialysis buffer comprises deionized water. In one embodiment, the polymer modified with crosslinkable groups is dialyzed in dialysis tubing with a molecular weight cutoff of between 2 kDa and 50 kDa. In one embodiment, the dialysis tubing has a molecular weight cutoff of between 2 kDa and 45 kDa. In one embodiment, the dialysis tubing has a molecular weight cutoff of between 2 kDa and 40 kDa. In one embodiment, the dialysis tubing has a molecular weight cutoff of between 2 kDa and 35 kDa. In one embodiment, the dialysis tubing has a molecular weight cutoff of between 2 kDa and 30 kDa. In one embodiment, the dialysis tubing has a molecular weight cutoff of between 2 kDa and 25 kDa. In one embodiment, the dialysis tubing has a molecular weight cutoff of between 2 kDa and 20 kDa. In one embodiment, the dialysis tubing has a molecular weight cutoff of between 8 kDa and 20 kDa. In one embodiment, the polymer modified with crosslinkable groups is dialyzed for between 12 hours and 10 days. In one embodiment, the polymer modified with crosslinkable groups is dialyzed for between 12 hours and 9.5 days. In one embodiment, the polymer modified with crosslinkable groups is dialyzed for between 12 hours and 9 days. In one embodiment, the polymer modified with crosslinkable groups is dialyzed for between 12 hours and 8.5 days. In one embodiment, the polymer modified with crosslinkable groups is dialyzed for between 12 hours and 8 days. In one embodiment, the polymer modified with crosslinkable groups is dialyzed for between 12 hours and 7.5 days. In one embodiment, the polymer modified with crosslinkable groups is dialyzed for between 12 hours and 7 days. In one embodiment, the polymer modified with crosslinkable groups is dialyzed for between 12 hours and 6.5 days. In one embodiment, the polymer modified with crosslinkable groups is dialyzed for between 12 hours and 6 days. In one embodiment, the polymer modified with crosslinkable groups is dialyzed for between 1 and 6 days. In one embodiment, the polymer modified with crosslinkable groups is dialyzed for between 2 and 6 days. In one embodiment, the polymer modified with crosslinkable groups is dialyzed for between 3 and 6 days. In one embodiment, the polymer modified with crosslinkable groups is dialyzed for between 4 and 6 days. In one embodiment, the dialysis buffer is heated during the dialysis of the polymer modified with crosslinkable groups. In one embodiment, the dialysis buffer is heated between 30° C. and 200° C. In one embodiment, the dialysis buffer is heated between 30° C. and 190° C. In one embodiment, the dialysis buffer is heated between 30° C. and 180° C. In one embodiment, the dialysis buffer is heated between 30° C. and 170° C. In one embodiment, the dialysis buffer is heated between 30° C. and 160° C. In one embodiment, the dialysis buffer is heated between 30° C. and 150° C. In one embodiment, the dialysis buffer is heated between 30° C. and 140° C. In one embodiment, the dialysis buffer is heated between 30° C. and 130° C. In one embodiment, the dialysis buffer is heated between 30° C. and 120° C. In one embodiment, the dialysis buffer is heated between 30° C. and 110° C. In one embodiment, the dialysis buffer is heated between 30° C. and 100° C. In one embodiment, the dialysis buffer is heated between 30° C. and 90° C. In one embodiment, the dialysis buffer is heated between 30° C. and 80° C. In one embodiment, the dialysis buffer is heated between 30° C. and 70° C. In one embodiment, the dialysis buffer is heated between 30° C. and 60° C. In one embodiment, the dialysis buffer is heated between 40° C. and 60° C. In one embodiment, the dialysis buffer is heated between 45° C. and 55° C.

In one embodiment, the dialyzed polymer modified with crosslinkable groups is filtered. In one embodiment, the filtered dialyzed polymer modified with crosslinkable groups is dried. In one embodiment, the filtered dialyzed polymer modified with crosslinkable groups is lyophilized. In one embodiment, the filtered dialyzed polymer modified with crosslinkable groups is lyophilized for 30 minutes to 10 days. In one embodiment, the filtered modified polymer is lyophilized for 30 minutes to 9.5 days. In one embodiment, the filtered modified polymer is lyophilized for 30 minutes to 9 days. In one embodiment, the filtered modified polymer is lyophilized for 30 minutes to 8.5 days. In one embodiment, the filtered modified polymer is lyophilized for 30 minutes to 8 days. In one embodiment, the filtered modified polymer is lyophilized for 30 minutes to 7.5 days. In one embodiment, the filtered modified polymer is lyophilized for 30 minutes to 7 days. In one embodiment, the filtered modified polymer is lyophilized for 30 minutes to 6.5 days. In one embodiment, the filtered modified polymer is lyophilized for 30 minutes to 6 days. In one embodiment, the filtered modified polymer is lyophilized for 30 minutes to 5.5 days. In one embodiment, the filtered modified polymer is lyophilized for 30 minutes to 5 days. In one embodiment, the filtered modified polymer is lyophilized for 1 to 5 days. In one embodiment, the filtered modified polymer is lyophilized for 2 to 5 days. In one embodiment, the filtered modified polymer is lyophilized for 3 to 5 days.

In some embodiments, the step providing a crosslinking agent further comprises step 122, wherein an antimicrobial agent is added to the crosslinking agent. The antimicrobial agent can be any antimicrobial agent known to those of skill in the art. Exemplary antimicrobial agents are described elsewhere herein. In one embodiment, the antimicrobial agent is an AMP. In one embodiment, the antimicrobial agent is dissolved in a solution comprising the crosslinking agent. In one embodiment, the (w/v) ratio of the crosslinking agent to antimicrobial agent is between about 150:1 to 1:1. In one embodiment, the (w/v) ratio of the crosslinking agent to antimicrobial agent is between about 140:1 to 1:1. In one embodiment, the (w/v) ratio of the crosslinking agent to antimicrobial agent is between about 130:1 to 1:1. In one embodiment, the (w/v) ratio of the crosslinking agent to antimicrobial agent is between about 120:1 to 1:1. In one embodiment, the (w/v) ratio of the crosslinking agent to antimicrobial agent is between about 110:1 to 1:1. In one embodiment, the (w/v) ratio of the crosslinking agent to antimicrobial agent is between about 100:1 to 1:1. In one embodiment, the (w/v) ratio of the crosslinking agent to antimicrobial agent is between about 90:1 to 1:1. In one embodiment, the (w/v) ratio of the crosslinking agent to antimicrobial agent is between about 80:1 to 1:1. In one embodiment, the (w/v) ratio of the crosslinking agent to antimicrobial agent is between about 70:1 to 1:1. In one embodiment, the (w/v) ratio of the crosslinking agent to antimicrobial agent is between about 70:1 to 4:1.

In step 130, the solution comprising a polymer comprising crosslinkable groups and the solution comprising a crosslinking agent are mixed to form a combined solution. In one embodiment, the polymer comprising crosslinkable groups comprises gelatin modified with crosslinkable groups. In one embodiment, the solution comprising the polymer comprising crosslinkable groups is added to the solution comprising the crosslinking agent. In one embodiment, the solution comprising a polymer comprising crosslinkable groups is added dropwise to the solution comprising the crosslinking agent. In one embodiment, the solution comprising a polymer comprising crosslinkable groups is added all at once to the solution comprising the crosslinking agent. In one embodiment, the solution comprising the crosslinking agent is added to the solution comprising a polymer comprising crosslinkable groups. In one embodiment, the solution comprising the crosslinking agent is added dropwise to the solution comprising a polymer comprising crosslinkable groups. In one embodiment, the solution comprising the crosslinking agent is added all at once to the solution comprising a polymer comprising crosslinkable groups. In one embodiment, the addition occurs at room temperature. In one embodiment, the solution comprising the crosslinking agent is heated to a temperature between 30° C. and 100° C. In one embodiment, the solution comprising a polymer comprising crosslinkable groups is heated to a temperature between 30° C. and 100° C. In one embodiment, the solution comprising the crosslinking agent is cooled to a temperature between 13° C. and −78° C. In one embodiment, the solution comprising a polymer comprising crosslinkable groups is cooled to a temperature between 13° C. and −78° C. In one embodiment, the solution comprising the crosslinking agent is stirred during the addition. In one embodiment, the solution comprising a polymer comprising crosslinkable groups is stirred during the addition. In one embodiment, the combined solution is stirred after the solutions of a polymer comprising crosslinkable groups and crosslinking agent are mixed.

In some embodiments, the step of mixing the polymer comprising crosslinkable groups and the solution comprising a crosslinking agent to form a combined solution further comprises step 132, wherein an osteoinductive agent is added to the combined solution. Exemplary osteoinductive agents are described elsewhere herein. In one embodiment, the osteoinductive agent comprises silicate nanoparticles. In one embodiment, the silicate nanoparticles comprise laponite nanoparticles. In one embodiment, the osteoinductive agent is dissolved in the combined solution. In one embodiment, the osteoinductive agent is dispersed in the combined solution. In one embodiment, the (w/v) ratio of the crosslinking agent to osteoinductive agent is between about 1500:1 to 1:2. In one embodiment, the (w/v) ratio of the crosslinking agent to osteoinductive agent is between about 1400:1 to 1:2. In one embodiment, the (w/v) ratio of the crosslinking agent to osteoinductive agent is between about 1300:1 to 1:2. In one embodiment, the (w/v) ratio of the crosslinking agent to osteoinductive agent is between about 1200:1 to 1:2. In one embodiment, the (w/v) ratio of the crosslinking agent to osteoinductive agent is between about 1100:1 to 1:2. In one embodiment, the (w/v) ratio of the crosslinking agent to osteoinductive agent is between about 1000:1 to 1:2. In one embodiment, the (w/v) ratio of the crosslinking agent to osteoinductive agent is between about 900:1 to 1:2. In one embodiment, the (w/v) ratio of the crosslinking agent to osteoinductive agent is between about 800:1 to 1:2. In one embodiment, the (w/v) ratio of the crosslinking agent to osteoinductive agent is between about 700:1 to 1:2. In one embodiment, the (w/v) ratio of the crosslinking agent to osteoinductive agent is between about 650:1 to 1:2.

In step 140, the combined solution is crosslinked. In one embodiment, the combined solution is photocrosslinked. In one embodiment, the combined solution is photocrosslinked using visible light. In one embodiment, the combined solution is photocrosslinked using UV light. In one embodiment, the solution is photocrosslinked using both UV and visible light. In one embodiment, the solution is photocrosslinked using light having of wavelength of between about 380 nm and 700 nm. In one embodiment, the solution is photocrosslinked using light having of wavelength of between about 380 nm and 650 nm. In one embodiment, the solution is photocrosslinked using light having of wavelength of between about 380 nm and 600 nm. In one embodiment, the solution is photocrosslinked using light having of wavelength of between about 380 nm and 550 nm. In one embodiment, the solution is photocrosslinked using light having of wavelength of between about 380 nm and 500 nm. In one embodiment, the solution is photocrosslinked using light having of wavelength of between about 400 nm and 500 nm. In one embodiment, the solution is photocrosslinked using a dental light curing unit. In one embodiment, the combined solution is irradiated with light between about 10 seconds and 30 minutes to photocrosslink the combined solution. In one embodiment, the combined solution is irradiated with light between about 10 seconds and 25 minutes. In one embodiment, the combined solution is irradiated with light between about 10 seconds and 20 minutes. In one embodiment, the combined solution is irradiated with light between about 10 seconds and 15 minutes. In one embodiment, the combined solution is irradiated with light between about 10 seconds and 10 minutes. In one embodiment, the combined solution is irradiated with light between about 1 and 5 minutes.

In one embodiment, the combined solution crosslinks through the formation of coordination complexes. In one embodiment, the crosslinkable groups on the modified biopolymer coordinate with metal^{2+/3+} ions to form coordination complexes. Exemplary crosslinkable groups are described elsewhere herein. Exemplary metal^{2+/3+} ions are described elsewhere herein.

### Method of Treatment

In one aspect, the present invention relates to inhibiting or reducing microbial growth in a subject's mouth. In one embodiment, the microbial growth is inhibited or reduced at the site of a dental implant. In one embodiment, the microbial growth is inhibited or reduced at the site of an oral bone graft. In one embodiment, the hydrogel inhibits or reduces microbial growth at the site of a dental implant. In one embodiment, the hydrogel inhibits or reduces microbial growth at the site of an oral bone graft. In one embodiment, the hydrogel comprises osteoinductive agents and functions as a bone graft while inhibiting or reducing microbial growth. In one embodiment, the hydrogel is formed in the patient's mouth.

In one embodiment, the method comprises (a) applying a solution comprising a hydrogel precursor and an antimicrobial agent to one or more surfaces of a subject's mouth to form a coating; and (b) crosslinking the coating to form a hydrogel.

In one embodiment, the solution of step (a) comprising a hydrogel precursor comprises a polymer comprising crosslinkable groups. Exemplary polymers are described elsewhere herein. In one embodiment, the polymer comprises gelatin. Exemplary crosslinkable groups are described elsewhere herein. In one embodiment, the crosslinkable groups comprise methacryloyl groups. In one embodiment, the crosslinkable groups comprise catechol groups. In one embodiment, the crosslinkable groups comprise both methacryloyl groups and catechol groups. In one embodiment, the hydrogel precursor comprises one or more crosslinking agents. In one embodiment, the crosslinking agent comprises a photoinitiator. Exemplary photoinitiators are described elsewhere herein. In one embodiment, the crosslinking agent comprises a metal^{2+/3+} ion. Exemplary metal^{2+/3+} ions are described elsewhere herein. In one embodiment, the crosslinking agent comprises both a photoinitiator and a metal^{2+/3+} ion.

The antimicrobial agent can be any antimicrobial agent known to a person of skill in the art. Exemplary antimicrobial agents are described elsewhere herein. In one embodiment, the antimicrobial agent is an AMP. In one embodiment, the antimicrobial agent is AMP Tet213.

The solution of step (a) comprising a hydrogel precursor and an antimicrobial agent can be applied to any surface of a subject's mouth. In one embodiment, the solution is applied a portion of the subject's gum. In one embodiment, the solution is applied to an implant pocket. In one embodiment, the solution is applied to the area around an existing implant present in the patient's mandible. In one embodiment, the solution is applied one or more of a subject's teeth.

In one embodiment, step (a) further comprises the step of inserting a dental implant or bone graft into the subject's mouth such that the dental implant or bone graft contacts the coating. In one embodiment, the dental implant comprises metal. In one embodiment, the dental implant comprises titanium.

In one embodiment, the dental implant or bone graft is also coated on one or more surfaces with the solution of step (a). In one embodiment, step (a) further comprises the step of inserting a dental implant or oral bone graft into the subject's mouth such that the coating on the dental implant or bone graft contacts the coating on one or more surfaces of a subject's mouth. In step (b), the hydrogel precursor is crosslinked to form a hydrogel. In one embodiment, the hydrogel precursor is photocrosslinked to form a hydrogel. Exemplary conditions for the photocrosslinking are described elsewhere herein. In one embodiment, the hydrogel precursor crosslinks by the formation of coordination complexes. In one embodiment, the coordination complexes form between the crosslinkable groups on the gelatin and metal^{2+/3+} ions. Exemplary crosslinkable groups are described elsewhere herein. Exemplary metal^{2+/3+} ions are described elsewhere herein.

In one embodiment, the hydrogel comprises photocrosslinked gelatin methacryloyl. In one embodiment, the hydrogel does not comprise an antimicrobial agent or silicate nanoparticles. In one embodiment, the step of the step of crosslinking the coating further comprises the step of adhering the dental implant to the subject's mouth. In one embodiment, the hydrogel that does not comprise antimicrobial agents or silicate nanoparticles (i.e. GelMA hydrogel) acts as an adhesive. In one embodiment, the GelMA hydrogel acts as an adhesive between a dental implant and the native tissue in a subject's mouth. In one embodiment, the adhesiveness of the GelMA hydrogel, as measured by wound closure tests (ASTM F2458-05), is between about 1 kPa to about 150 kPa. In one embodiment, the adhesiveness of the GelMA hydrogel, as measured by wound closure tests, is between about 1 kPa to about 140 kPa. In one embodiment, the adhesiveness of the GelMA hydrogel, as measured by wound closure tests, is between about 1 kPa to about 130 kPa. In one embodiment, the adhesiveness of the GelMA hydrogel, as measured by wound closure tests, is between about 1 kPa to about 120 kPa. In one embodiment, the adhesiveness of the GelMA hydrogel, as measured by wound closure tests, is between about 1 kPa to about 110 kPa. In one embodiment, the adhesiveness of the GelMA hydrogel, as measured by wound closure tests, is between about 1 kPa to about 100 kPa. In one embodiment, the adhesiveness of the GelMA hydrogel, as measured by wound closure tests, is between about 1 kPa to about 90 kPa. In one embodiment, the adhesiveness of the GelMA hydrogel, as measured by wound closure tests, is between about 1 kPa to about 80 kPa. In one embodiment, the adhesiveness of the GelMA hydrogel, as measured by wound closure tests, is between about 1 kPa to about 70 kPa. In one embodiment, the adhesiveness of the GelMA hydrogel, as measured by wound closure tests, is between about 10 kPa to about 70 kPa. In one embodiment, the adhesiveness of the GelMA hydrogel, as measured by wound closure tests, is between about 15 kPa to about 68 kPa. In one embodiment, the adhesiveness of the GelMA hydrogel, as measured by lap shear tests (ASTM F2255-05), is between about 1 kPa and 150 kPa. In one embodiment, the adhesiveness of the GelMA hydrogel, as measured by lap shear tests is between about 1 kPa and 140 kPa. In one embodiment, the adhesiveness of the GelMA hydrogel, as measured by lap shear tests, is between about 1 kPa and 130 kPa. In one embodiment, the adhesiveness of the GelMA hydrogel, as measured by lap shear tests, is between about 1 kPa and 120 kPa. In one embodiment, the adhesiveness of the GelMA hydrogel, as measured by lap shear tests, is between about 1 kPa and 110 kPa. In one embodiment, the adhesiveness of the GelMA hydrogel, as measured by lap shear tests, is between about 1 kPa and 100 kPa. In one embodiment, the adhesiveness of the GelMA hydrogel, as measured by lap shear tests, is between about 1 kPa and 90 kPa. In one embodiment, the adhesiveness of the GelMA hydrogel, as measured by lap shear tests, is between about 1 kPa and 80 kPa. In one embodiment, the adhesiveness of the GelMA hydrogel, as measured by lap shear tests, is between about 10 kPa and 80 kPa. In one embodiment, the adhesiveness of the GelMA hydrogel, as measured by lap shear tests, is between about 15 kPa and 65 kPa. In one embodiment, the adhesiveness of the GelMA hydrogel, as measure by burst pressure tests (ASTM F2392-04), is between about 1 kPa and 150 kPa. In one embodiment, the adhesiveness of the GelMA hydrogel, as measure by burst pressure tests, is between about 1 kPa and 140 kPa. In one embodiment, the adhesiveness of the GelMA hydrogel, as measure by burst pressure tests, is between about 1 kPa and 130 kPa. In one embodiment, the adhesiveness of the GelMA hydrogel, as measure by burst pressure tests, is between about 1 kPa and 120 kPa. In one embodiment, the adhesiveness of the GelMA hydrogel, as measure by burst pressure tests, is between about 1 kPa and 110 kPa. In one embodiment, the adhesiveness of the GelMA hydrogel, as measure by burst pressure tests, is between about 1 kPa and 100 kPa. In one embodiment, the adhesiveness of the GelMA hydrogel, as measure by burst pressure tests, is between about 1 kPa and 90 kPa. In one embodiment, the adhesiveness of the GelMA hydrogel, as measure by burst pressure tests, is between about 1 kPa and 80 kPa. In one embodiment, the adhesiveness of the GelMA hydrogel, as measure by burst pressure tests, is between about 1 kPa and 70 kPa. In one embodiment, the adhesiveness of the GelMA hydrogel, as measure by burst pressure tests, is between about 1 kPa and 60 kPa. In one embodiment, the adhesiveness of the GelMA hydrogel, as measure by burst pressure tests, is between about 1 kPa and 50 kPa. In one embodiment, the adhesiveness of the GelMA hydrogel, as measure by burst pressure tests, is between about 10 kPa and 50 kPa. In one embodiment, the adhesiveness of the GelMA hydrogel, as measure by burst pressure tests, is between about 15 kPa and 45 kPa.

In one embodiment, the hydrogel dental implant adhesive comprises gelatin methacryloyl modified with catechol groups. In one embodiment, the methacryloyl groups have been photocrosslinked and the catechol groups are coordinated with metal^{2+/3+} ions, forming GelMAC. In one embodiment, the GelMAC hydrogel dental implant adhesive does not comprise an antimicrobial agent or silicate nanoparticles. In one embodiment, the GelMAC hydrogel acts as an adhesive. In one embodiment, the GelMAC hydrogel acts as an adhesive between a dental implant and the native tissue in a subject's mouth. In one embodiment, the adhesiveness of the GelMAC hydrogel, as measured by wound closure tests (ASTM F2458-05), is between about 1 kPa to about 250 kPa. In one embodiment, the adhesiveness of the GelMAC hydrogel, as measured by wound closure tests, is between about 1 kPa to about 230 kPa. In one embodiment, the adhesiveness of the GelMAC hydrogel, as measured by wound closure tests, is between about 1 kPa to about 210 kPa. In one embodiment, the adhesiveness of the GelMAC hydrogel, as measured by wound closure tests, is between about 1 kPa to about 190 kPa. In one embodiment, the adhesiveness of the GelMAC hydrogel, as measured by wound closure tests, is between about 1 kPa to about 180 kPa. In one embodiment, the adhesiveness of the GelMAC hydrogel, as measured by wound closure tests, is between about 10 kPa to about 180 kPa. In one embodiment, the adhesiveness of the GelMAC hydrogel, as measured by wound closure tests, is between about 30 kPa to about 180 kPa. In one embodiment, the adhesiveness of the GelMAC hydrogel, as measured by wound closure tests, is between about 40 kPa to about 180 kPa. In one embodiment, the adhesiveness of the GelMAC hydrogel, as measure by burst pressure tests (ASTM F2392-04), is between about 1 kPa and 150 kPa. In one embodiment, the adhesiveness of the GelMAC hydrogel, as measure by burst pressure tests, is between about 1 kPa and 130 kPa. In one embodiment, the adhesiveness of the GelMAC hydrogel, as measure by burst pressure tests, is between about 1 kPa and 110 kPa. In one embodiment, the adhesiveness of the GelMAC hydrogel, as measure by burst pressure tests, is between about 1 kPa and 90 kPa. In one embodiment, the adhesiveness of the GelMAC hydrogel, as measure by burst pressure tests, is between about 1 kPa and 70 kPa. In one embodiment, the adhesiveness of the GelMAC hydrogel, as measure by burst pressure tests, is between about 1 kPa and 50 kPa. In one embodiment, the adhesiveness of the GelMAC hydrogel is dependent upon the concentration of metal coordinated to the catechol groups.

In one embodiment, the hydrogel dental implant adhesive comprises photocrosslinked gelatin methacryloyl, an AMP antimicrobial agent, and does not comprise silicate nanoparticles (i.e. GelAMP). In one embodiment, the GelAMP hydrogel acts as an adhesive. In one embodiment, the GelAMP hydrogel acts as an adhesive between a dental implant and the native tissue in a subject's mouth. In one embodiment, the adhesiveness of the GelAMP hydrogel, as measured by wound closure tests (ASTM F2458-05), is between about 1 kPa to about 150 kPa. In one embodiment, the adhesiveness of the GelAMP hydrogel, as measured by wound closure tests, is between about 1 kPa to about 130 kPa. In one embodiment, the adhesiveness of the GelAMP hydrogel, as measured by wound closure tests, is between about 1 kPa to about 110 kPa. In one embodiment, the adhesiveness of the GelAMP hydrogel, as measured by wound closure tests, is between about 1 kPa to about 90 kPa. In one embodiment, the adhesiveness of the GelAMP hydrogel, as measured by wound closure tests, is between about 1 kPa to about 70 kPa. In one embodiment, the adhesiveness of the GelAMP hydrogel, as measured by wound closure tests, is between about 1 kPa to about 60 kPa. In one embodiment, the adhesiveness of the GelAMP hydrogel, as measured by wound closure tests, is between about 20 kPa to about 60 kPa. In one embodiment, the adhesiveness of the GelAMP hydrogel, as measured by lap shear tests (ASTM F2255-05), is between about 1 kPa to about 150 kPa. In one embodiment, the adhesiveness of the GelAMP hydrogel, as measured by lap shear tests, is between about 1 kPa to about 130 kPa. In one embodiment, the adhesiveness of the GelAMP hydrogel, as measured by lap shear tests, is between about 1 kPa to about 110 kPa. In one embodiment, the adhesiveness of the GelAMP hydrogel, as measured by lap shear tests, is between about 1 kPa to about 90 kPa. In one embodiment, the adhesiveness of the GelAMP hydrogel, as measured by lap shear tests, is between about 1 kPa to about 80 kPa. In one embodiment, the adhesiveness of the GelAMP hydrogel, as measured by lap shear tests, is between about 20 kPa to about 80 kPa. In one embodiment, the adhesiveness of the GelAMP hydrogel, as measured by lap shear tests, is between about 40 kPa to about 80 kPa. In one embodiment, the adhesiveness of the GelAMP hydrogel, as measured by burst pressure tests (ASTM F2392-04), is between about 1 kPa to about 150 kPa. In one embodiment, the adhesiveness of the GelAMP hydrogel, as measured by burst pressure tests, is between about 1 kPa to about 130 kPa. In one embodiment, the adhesiveness of the GelAMP hydrogel, as measured by burst pressure tests, is between about 1 kPa to about 110 kPa. In one embodiment, the adhesiveness of the GelAMP hydrogel, as measured by burst pressure tests, is between about 1 kPa to about 90 kPa. In one embodiment, the adhesiveness of the GelAMP hydrogel, as measured by burst pressure tests, is between about 1 kPa to about 70 kPa. In one embodiment, the adhesiveness of the GelAMP hydrogel, as measured by burst pressure tests, is between about 1 kPa to about 50 kPa. In one embodiment, the adhesiveness of the GelAMP hydrogel, as measured by burst pressure tests, is between about 20 kPa to about 50 kPa. In one embodiment, the adhesiveness of the GelAMP hydrogel is dependent on the concentration of AMP in the hydrogel.

In one embodiment, the hydrogel dental implant adhesive comprises photocrosslinked gelatin methacryloyl, silicate nanoparticles, and does not comprise an antimicrobial agent (i.e. GelMA w/ SN). In one embodiment, the GelMA w/ SN hydrogel acts as an adhesive. In one embodiment, the GelMA w/ SN hydrogel acts as an adhesive between a dental implant and the native tissue in a subject's mouth. In one embodiment, the adhesiveness of the GelMA w/ SN hydrogel, as measured by wound closure tests (ASTM F2458-05), is between about 1 kPa to about 300 kPa. In one embodiment, the adhesiveness of the GelMA w/ SN hydrogel, as measured by wound closure tests, is between about 1 kPa to about 280 kPa. In one embodiment, the adhesiveness of the GelMA w/ SN hydrogel, as measured by wound closure tests, is between about 1 kPa to about 260 kPa. In one embodiment, the adhesiveness of the GelMA w/ SN hydrogel, as measured by wound closure tests, is between about 1 kPa to about 240 kPa. In one embodiment, the adhesiveness of the GelMA w/ SN hydrogel, as measured by wound closure tests, is between about 1 kPa to about 220 kPa. In one embodiment, the adhesiveness of the GelMA w/ SN hydrogel, as measured by wound closure tests, is between about 1 kPa to about 200 kPa. In one embodiment, the adhesiveness of the GelMA w/ SN hydrogel, as measured by wound closure tests, is between about 1 kPa to about 180 kPa. In one embodiment, the adhesiveness of the GelMA w/ SN hydrogel, as measured by wound closure tests, is between about 1 kPa to about 160 kPa. In one embodiment, the adhesiveness of the GelMA w/ SN hydrogel, as measured by wound closure tests, is between about 1 kPa to about 140 kPa. In one embodiment, the adhesiveness of the GelMA w/ SN hydrogel, as measured by wound closure tests, is between about 1 kPa to about 120 kPa. In one embodiment, the adhesiveness of the GelMA w/ SN hydrogel, as measured by wound closure tests, is between about 1 kPa to about 100 kPa. In one embodiment, the adhesiveness of the GelMA w/ SN hydrogel, as measured by wound closure tests, is between about 20 kPa to about 100 kPa. In one embodiment, the adhesiveness of the GelMA w/ SN hydrogel, as measured by lap shear tests (ASTM F2255-05), is between about 1 kPa to about 400 kPa. In one embodiment, the adhesiveness of the GelMA w/ SN hydrogel, as measured by lap shear tests, is between about 1 kPa to about 380 kPa. In one embodiment, the adhesiveness of the GelMA w/ SN hydrogel, as measured by lap shear tests, is between about 1 kPa to about 360 kPa. In one embodiment, the adhesiveness of the GelMA w/ SN hydrogel, as measured by lap shear tests, is between about 1 kPa to about 340 kPa. In one embodiment, the adhesiveness of the GelMA w/ SN hydrogel, as measured by lap shear tests, is between about 1 kPa to about 320 kPa. In one embodiment, the adhesiveness of the GelMA w/ SN hydrogel, as measured by lap shear tests, is between about 1 kPa to about 300 kPa. In one embodiment, the adhesiveness of the GelMA w/ SN hydrogel, as measured by lap shear tests, is between about 1 kPa to about 280 kPa. In one embodiment, the adhesiveness of the GelMA w/ SN hydrogel, as measured by lap shear tests, is between about 1 kPa to about 260 kPa. In one embodiment, the adhesiveness of the GelMA w/ SN hydrogel, as measured by lap shear tests, is between about 1 kPa to about 240 kPa. In one embodiment, the adhesiveness of the GelMA w/ SN hydrogel, as measured by lap shear tests, is between about 1 kPa to about 230 kPa. In one embodiment, the adhesiveness of the GelMA w/ SN hydrogel, as measured by lap shear tests, is between about 20 kPa to about 230 kPa. In one embodiment, the adhesiveness of the GelMA w/ SN hydrogel is dependent upon the concentration of silicate nanoparticles in the hydrogel.

In one embodiment, the hydrogel dental implant adhesive comprises photocrosslinked gelatin methacryloyl, silicate nanoparticles, and an AMP antimicrobial agent (i.e. GelAMP w/ SN). In one embodiment, the GelAMP w/ SN hydrogel acts as an adhesive. In one embodiment, the GelAMP w/ SN hydrogel acts as an adhesive between a dental implant and the subject's mouth. In one embodiment, the GelAMP w/ SN hydrogel acts as an adhesive between a dental implant and the native tissue in a subject's mouth. In one embodiment, the adhesiveness of the GelAMP w/ SN hydrogel, as measured by wound closure tests (ASTM F2458-05), is between about 1 kPa to about 300 kPa. In one embodiment, the adhesiveness of the GelAMP w/ SN hydrogel, as measured by wound closure tests, is between about 1 kPa to about 280 kPa. In one embodiment, the adhesiveness of the GelAMP w/ SN hydrogel, as measured by wound closure tests, is between about 1 kPa to about 260 kPa. In one embodiment, the adhesiveness of the GelAMP w/ SN hydrogel, as measured by wound closure tests, is between about 1 kPa to about 240 kPa. In one embodiment, the adhesiveness of the GelAMP w/ SN hydrogel, as measured by wound closure tests, is between about 1 kPa to about 220 kPa. In one embodiment, the adhesiveness of the GelAMP w/ SN hydrogel, as measured by wound closure tests, is between about 1 kPa to about 200 kPa. In one embodiment, the adhesiveness of the GelAMP w/ SN hydrogel, as measured by wound closure tests, is between about 1 kPa to about 180 kPa. In one embodiment, the adhesiveness of the GelAMP w/ SN hydrogel, as measured by wound closure tests, is between about 1 kPa to about 160 kPa. In one embodiment, the adhesiveness of the GelAMP w/ SN hydrogel, as measured by wound closure tests, is between about 1 kPa to about 140 kPa. In one embodiment, the adhesiveness of the GelAMP w/ SN hydrogel, as measured by wound closure tests, is between about 20 kPa to about 140 kPa. In one embodiment, the adhesiveness of the GelAMP w/ SN hydrogel, as measured by wound closure tests, is between about 40 kPa to about 140 kPa. In one embodiment, the adhesiveness of the GelAMP w/ SN hydrogel, as measured by wound closure tests, is between about 60 kPa to about 140 kPa. In one embodiment, the adhesiveness of the GelAMP w/ SN hydrogel is dependent upon the concentration of AMP in the hydrogel. In one embodiment, the adhesiveness of the GelAMP w/ SN hydrogel is dependent upon the concentration of silicate nanoparticles in the hydrogel. In one embodiment, the adhesiveness of the GelAMP w/ SN hydrogel is dependent upon both the concentration of AMP and the concentration of silicate nanoparticles in the hydrogel.

In one embodiment, the method of inhibiting or reducing microbial growth further comprises a method of treating or preventing PIDs. In one embodiment, the method of treating or preventing PIDs comprises steps (a) and (b) described elsewhere herein for inhibiting or reducing microbial growth. In one embodiment, the PID is PI. In one embodiment, step (b) comprises crosslinking the coating to form a hydrogel. In one embodiment, the hydrogel is effective against one or more types of bacteria. In one embodiment, the hydrogel is effective against Gram positive bacteria. In one embodiment, the hydrogel is effective against Gram negative bacteria. In one embodiment, the hydrogel is effective against both Gram positive and Gram negative bacteria. Exemplary bacteria include, but are not limited to, Eubacterium nodatum, E. brachy, E. saphenum, Filifactor alocis, Slackia exigua, Parascardovia denticolens, Prevotella intermedia, Fusobacterium nucleatum, Porphyromonas gingivalis, Centipeda periodontii, Parvimonas micra, Prevotella buccae, Prevotella oralis, Prevotella melaninogenica, Prevotella denticola, Prevotella nigrescens, Tannerella forsythia, and Treponema denticola. In one embodiment, the method of treating or preventing PIDs comprises the step of reducing the amount of bacteria at the site of the dental implant. In one embodiment, the method of treating or preventing PIDs comprises the step of inhibiting the growth of bacteria at the site of the dental implant. In one embodiment, the dental implant is an existing dental implant wherein there is an infection at the site of the implant.

In one embodiment, one or more AMPs provide the antimicrobial properties of the hydrogel. In one embodiment, the AMPs reduce the level of bacteria at the site of the implant 0.5-fold to 50-fold. In one embodiment, the AMPs reduce the level of bacteria at the site of the implant 0.5-fold to 45-fold. In one embodiment, the AMPs reduce the level of bacteria at the site of the implant 0.5-fold to 40-fold. In one embodiment, the AMPs reduce the level of bacteria at the site of the implant 0.5-fold to 35-fold. In one embodiment, the AMPs reduce the level of bacteria at the site of the implant 0.5-fold to 30-fold. In one embodiment, the AMPs reduce the level of bacteria at the site of the implant 0.5-fold to 25-fold. In one embodiment, the AMPs reduce the level of bacteria at the site of the implant 0.5-fold to 20-fold. In one embodiment, the AMPs reduce the level of bacteria at the site of the implant 0.5-fold to 15-fold. In one embodiment, the AMPs reduce the level of bacteria at the site of the implant 0.5-fold to 10-fold. In one embodiment, the reduction in bacteria levels is dependent upon the concentration of AMPs in the hydrogel.

In one embodiment, the method of inhibiting or reducing microbial growth further comprises a method of increasing the proliferation of cells at the site of the implant. In one embodiment, the method of increasing the proliferation of cells comprises steps (a) and (b) described elsewhere herein for inhibiting or reducing microbial growth. In one embodiment, the increase in proliferation of cells at the site of the implant promotes bone growth. In one embodiment, the increase of cell proliferation aids in the biointegration of the dental implant to the native tissue. In one embodiment, the increase of cell proliferation aids in the healing process at the site of the dental implant. In one embodiment, one or more AMPs affect the increase in the proliferation of cells. In one embodiment, one or more silicate nanoparticles affect the increase in the proliferation of cells. In one embodiment, both the one or more silicate nanoparticles and the one or more AMPs affect the increase in the proliferation of cells. In one embodiment, the metabolic activity of the cells near the implant site increases 0.5-fold to 100-fold. In one embodiment, the metabolic activity of the cells near the implant site increases 0.5-fold to 90-fold. In one embodiment, the metabolic activity of the cells near the implant site increases 0.5-fold to 80-fold. In one embodiment, the metabolic activity of the cells near the implant site increases 0.5-fold to 70-fold. In one embodiment, the metabolic activity of the cells near the implant site increases 0.5-fold to 60-fold. In one embodiment, the metabolic activity of the cells near the implant site increases 0.5-fold to 50-fold. In one embodiment, the metabolic activity of the cells near the implant site increases 0.5-fold to 40-fold. In one embodiment, the metabolic activity of the cells near the implant site increases 0.5-fold to 30-fold. In one embodiment, the metabolic activity of the cells near the implant site increases 0.5-fold to 20-fold. In one embodiment, the metabolic activity of the cells near the implant site increases 2-fold to 12-fold. In one embodiment, the metabolic activity of the cells near the implant site is dependent on the concentration of AMPs in the hydrogel. In one embodiment, the metabolic activity of the cells near the implant site is dependent on the concentration of silicate nanoparticles in the hydrogel. In one embodiment, the metabolic activity of the cells near the implant site is dependent on the concentration of both the AMPs and the silicate nanoparticles in the hydrogel.

In one embodiment, the method of inhibiting or reducing microbial growth further comprises a method of increasing the differentiation of cells at the site of the implant. In one embodiment, the hydrogel increases the differentiation of cells at the site of an existing implant. In one embodiment, one or more AMPs affect the increase in the differentiation of cells. In one embodiment, one or more silicate nanoparticles affect the increase in the differentiation of cells. In one embodiment, both the one or more silicate nanoparticles and the one or more AMPs affect the increase in the differentiation of cells. In one embodiment, cell differentiation increases 0.5-fold to 50-fold. In one embodiment, cell differentiation increases 0.5-fold to 45-fold. In one embodiment, cell differentiation increases 0.5-fold to 40-fold. In one embodiment, cell differentiation increases 0.5-fold to 35-fold. In one embodiment, cell differentiation increases 0.5-fold to 30-fold. In one embodiment, cell differentiation increases 0.5-fold to 25-fold. In one embodiment, cell differentiation increases 0.5-fold to 20-fold. In one embodiment, cell differentiation increases 0.5-fold to 15-fold. In one embodiment, cell differentiation increases 0.5-fold to 10-fold. In one embodiment, cell differentiation increases 0.5-fold to 5-fold. In one embodiment, the differentiation of the cells near the implant site is dependent on the concentration of AMPs in the hydrogel. In one embodiment, the differentiation of the cells near the implant site is dependent on the concentration of silicate nanoparticles in the hydrogel. In one embodiment, the differentiation of the cells near the implant site is dependent on the concentration of both the AMPs and the silicate nanoparticles in the hydrogel.

In one embodiment, the method of inhibiting or reducing microbial growth further comprises a method of inducing bone growth at the site of the implant. In one embodiment, the one or more AMPs induce bone growth at the site of the implant. In one embodiment, the one or more silicate nanoparticles induce bone growth at the site of the implant. In one embodiment, both the one or more silicate nanoparticles and the one or more AMPs induce bone growth at the site of the implant. In one embodiment, the bone growth comprises an increase in bone surface area. In one embodiment, the bone growth comprises an increase in bone volume. In one embodiment, the bone surface area increases 0.1-fold to 50-fold. In one embodiment, the bone surface area increases 0.1-fold to 45-fold. In one embodiment, the bone surface area increases 0.1-fold to 40-fold. In one embodiment, the bone surface area increases 0.1-fold to 35-fold. In one embodiment, the bone surface area increases 0.1-fold to 30-fold. In one embodiment, the bone surface area increases 0.1-fold to 25-fold. In one embodiment, the bone surface area increases 0.1-fold to 20-fold. In one embodiment, the bone surface area increases 0.1-fold to 15-fold. In one embodiment, the bone surface area increases 0.1-fold to 10-fold. In one embodiment, the bone surface area increases 0.1-fold to 5-fold. In one embodiment, the bone surface area increases 0.5-fold to 5-fold. In one embodiment, the bone volume increases 0.1-fold to 50-fold. In one embodiment, the bone volume increases 0.1-fold to 45-fold. In one embodiment, the bone surface area increases 0.1-fold to 40-fold. In one embodiment, the bone volume increases 0.1-fold to 35-fold. In one embodiment, the bone volume increases 0.1-fold to 30-fold. In one embodiment, the bone volume increases 0.1-fold to 25-fold. In one embodiment, the bone volume increases 0.1-fold to 20-fold. In one embodiment, the bone volume increases 0.1-fold to 15-fold. In one embodiment, the bone volume increases 0.1-fold to 10-fold. In one embodiment, the bone volume increases 0.1-fold to 5-fold. In one embodiment, the bone volume increases 0.5-fold to 5-fold. In one embodiment, the induction of bone growth is dependent on the concentration of AMPS in the hydrogel. In one embodiment, the induction of bone growth is dependent on the concentration of silicate nanoparticles in the adhesive hydrogel. In one embodiment, the induction of bone growth is dependent on the concentration of both the AMPs and the silicate nanoparticles in the hydrogel.

In another aspect, the present invention relates to a method of promoting bone formation. In one embodiment, the method comprises (a) applying a solution comprising a hydrogel precursor and an osteoinductive agent to one or more bone defects in a subject to form a coating; and (b) crosslinking the coating. In one embodiment, the hydrogel precursor comprises a crosslinking agent. Exemplary hydrogel precursors, crosslinking agents, and osteoinductive agents are described elsewhere herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1. An Antimicrobial Dental Light Curable Bioadhesive Hydrogel for Treatment of Peri-Implant Diseases

### Materials and Methods

Synthesis of gelatin methacryloyl (GelMA)

Hydrogels were synthesized using the highly cytocompatible and visible-light activated polymer gelatin methacryloyl, a chemically modified form of hydrolyzed collagen that possesses a high number of cell binding motifs and matrix-metalloproteinase (MMP) degradation sites (Assmann, A. et al., Biomaterials, 2017, 140:115-127). These characteristics are critical to ensure proper cell attachment and colonization of the scaffold.

Gelatin methacryloyl was synthesized as previously described (Assmann, A. et al., Biomaterials, 2017, 140:115-127; Noshadi, I. et al., Biomater. Sci., 2017, 5: 2093-2105). Briefly, 10 g gelatin from cold water fish skin (Sigma-Aldrich) was dissolved in 100 ml DPBS at 60° C. for 30 min. Next, 8% (v/v) methacrylic anhydride (Sigma-Aldrich) was added to the solution drop-wise under vigorous stirring at 60° C. for another 3 h. The solution was then diluted with 300 ml DPBS to stop the reaction and dialyzed (Spectrum Laboratories, MWCO=12-14 kDa) in a deionized water bath at 50° C. for 5 days to remove the unreacted methacrylic anhydride. The resulting solution was filtered and lyophilized for 4 days.

### Fabrication of Bioadhesives

Gel adhesive hydrogels were formed by first dissolving different concentrations of gelatin methacryloyl (7 and 15% (w/v)) in the photoinitiator solution containing triethanolamine (TEA, 1.88% (w/v)) and N-vinyl caprolactam (VC, 1.25% (w/v)) in distilled water at room temperature. A separate solution of Eosin Y disodium salt (0.5 mM) was also prepared in distilled water. The biopolymer/TEA/VC solutions were then mixed with Eosin Y prior to crosslinking to form the final precursor solution. To form the hydrogels, 70 mL of the precursor solution was pipetted into polydimethylsiloxane (PDMS) cylindrical molds (diameter: 6 mm; height: 2.5 mm) for compressive tests, or rectangular molds (12×5×1 mm) for tensile tests. Lastly, the solutions were photocrosslinked upon exposure to visible light (420-480 nm) for 120 s, using a VALO dental light curing unit (Ultradent Products, Inc.).

The GelAMP bioadhesives were synthesized based on the combination of biocompatible photoinitiators (triethanolamine (TEA)/N-vinyl caprolactam (VC)/Eosin Y), a naturally-derived gelatin-based biopolymer (gelatin methacryloyl), and an antimicrobial peptide (AMP Tet213). To form the antimicrobial hydrogels, different concentrations of AMP Tet213 (CSC Scientific, Inc.) (0.1-1% (w/v)) were dissolved in TEA/VC/Eosin Y photoinitiator solution.

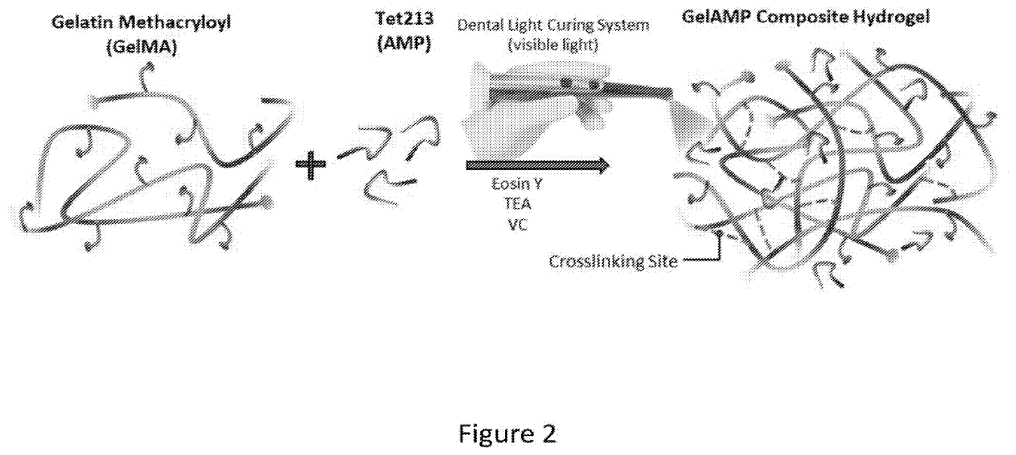

GelAMP hydrogels were preferably formed by dissolving 0.2% (w/v) AMP Tet213 (CSC Scientific, Inc.) in TEA/VC/Eosin Y photoinitiator solution. The lyophilized biopolymers were then dissolved in the resulting solution and photocrosslinked as described before. To form the antimicrobial GelAMP bioadhesives, the GelMA prepolymers were dissolved at various concentrations (7% and 15%) in a photoinitiator solution containing AMP Tet213 (0.2% (w/v), or 1.34 mM) and photocrosslinked using a dental curing light (420-480 nm) (FIG. 2). Control hydrogels, Gel, were formed using a similar technique, but without incorporation of AMP.

Type I or cleavage-type initiators are widely used in tissue engineering and are designed to be activated within the range of UV wavelength (i.e. 360-400 nm). However, exposure to UV light could lead to cell and damage (Kielbassa, C. et al., Carcinogenesis, 1997, 18:811-816; de Gruijl, F. R. et al., Journal of Photochemistry and Photobiology B: Biology, 2001, 63:19-27; Sinha, R. P. et al., Photochemical & Photobiological Sciences, 2002, 1:225-237), impair cellular function (Kappes, U. P. et al., Journal of Investigative Dermatology, 2006, 126, 667-675; Jones, C. A. et al., Radiation Research, 1987, 110:244-254), and even lead to neoplasia and cancer (Armstrong, B. K. et al., Journal of Photochemistry and Photobiology B: Biology, 2001, 63:8-18; Wang, Z. et al., Biofabrication, 2015, 7:045009). Moreover, only a few type I photoinitiators such as 2-Hydroxy-4′-(2-hydroxyethoxy)-2-methylpropiophenone (Irgacure-2959) and Lithium phenyl-2,4,6-trimethylbenzoylphosphinate (LAP) have been shown to be cytocompatible at low concentrations (Monteiro, N. et al., Dental Materials, 2018, 34, 389-399; Wang, Z. et al., Biofabrication, 2015, 7:045009; Assmann, A. et al., Biomaterials, 2017, 140:115-127; Soucy, J. R. et al., Tissue Engineering Part A, 2018, 24:1393-1405). However, Irgacure-2959 has low water solubility and cannot be activated with visible light since its molar absorptivity is limited in the visible light range (wavelengths>400 nm). Although LAP has high water solubility and cytocompatibility, its highest molar absorbance is in UV range wavelengths (365-385 nm, ε≈150-230 M⁻¹ cm⁻¹), which limits its activation in the visible light range (ε≈30 M⁻¹ cm⁻¹ at 405 nm) (Shih, H. et al., Macromolecular Rapid Communications, 2013, 34:269-273). Considering the effective wavelength of Food and Drug Administration (FDA) approved dental curing light systems (420-480 nm), cleavage-type photoinitiators have limited potential to be used with these platforms in the clinical setting. To address these limitations, a visible light activated photoinitiator, Eosin Y, which is known as Type II or noncleavage-type photoinitiator was used. This photoinitiator not only can minimize the safety concerns associated with UV light, but also can be rapidly activated with wavelengths (420-480 nm, a ε>50000 M⁻¹ cm⁻¹) produced by commercial dental curing systems ((Shih, H. et al., Macromolecular Rapid Communications, 2013, 34:269-273; Noshadi, I. et al., Biomater. Sci., 2017, 5:2093-2105). TEA and VC were used as a co-initiator and a co-monomer respectively, to assist free radical photoinitiation (Noshadi, I. et al., Biomater. Sci., 2017, 5:2093-2105).

### Mechanical Characterization

The tensile and compressive properties of the hydrogel adhesives were evaluated using an Instron 5542 mechanical tester, as described previously (Shirzaei, E. et al., ACS Biomaterials Science & Engineering, 2018, 4:2528-2540). For tensile tests, rectangular samples were fixed between two pieces of double-sided tape, placed within the Instron grips, and then extended at a rate of 1 mm/min until failure. Next, using the Bluehill 3 software, the tensile strain (mm/mm) and stress (kPa) were determined, and the elastic moduli of the samples was calculated from the slope of the stress-strain curves. For the compressive tests, hydrogels were loaded between compression platens immersed in a DPBS bath and then compressed at a rate of 1 mm/min until 70% strain. The compressive strain (mm/mm) and stress (kPa) were determined and the slope of the linear region (0.05-0.2 mm/mm strain) on the stress (kPa)/strain (mm/mm) curve was determined to calculate the compressive modulus (n≥4).

### In Vitro Swellability and Degradation Studies

The in vitro swellability (24 h) and degradation (14 days) of bioadhesives were performed in DPBS as described previously (Shirzaei, E. et al., ACS Biomaterials Science & Engineering, 2018, 4:2528-2540). For the swellability studies, cylindrical hydrogels were prepared and weighed, followed by immersion in DPBS for 24 h. The weight of the swollen gels was measured at different time points and the swelling ratio was calculated using the following equation, where SR is the swelling ratio, W_{S} is the swollen weight of the hydrogel, and Wo is the the initial weight before swelling (n≥4):

$$S R = \frac{{W}_{S} - {W}_{0}}{{W}_{0}} .$$

For the degradation studies, cylindrical hydrogels were prepared, lyophilized, and their initial dry weight was recorded. Next, the samples were immersed in collagenase solution in DPBS (20 μg/ml) and incubated at 37° C. for up to 14 days. The solution was replaced every three days. On days 1, 4, 7, and 14 post-incubation, the samples were removed from the solutions, and lyophilized again. The final weight of the samples was then recorded, and the percentage of the weight loss was considered as degradation.

### In Vitro Adhesion Studies

In vitro wound closure: Wound closure was performed on both porcine gingiva and skin using a modified ASTM F2458-05 test, as described previously (Shirzaei, E. et al., ACS Biomaterials Science & Engineering, 2018, 4:2528-2540). Briefly, the porcine gingiva was isolated from fresh porcine mandible. Tissues were then cut into 1×2 cm pieces and kept moist prior to the test. The tissues were glued onto two pre-cut glass slides (20 mm×30 mm) and then 50 mL of precursor solution was pipetted and crosslinked using a dental light curing system to form the adhesives. The samples were then placed between the Instron tensile grips and the ultimate adhesive strength was calculated at break (n≥5).

In vitro lap shear: The lap shear strength of the bioadhesives and two commercial adhesives Evicel® (Ethicon, Somerville, N.J., USA) and CoSEAL™ (Baxter, Deerfield, Ill., USA) was determined according to a modified ASTM test (F2255-05). Both titanium and glass slides were used as the substrates. Glass slides (10 mm×30 mm) were coated with gelatin solution and dried at 37° C. For adhesive tests on titanium, a piece of titanium (10 mm×10 mm) was attached to a glass slide and 10 μl of the precursor solution was photocrosslinked between the titanium and the gelatin coated glass slide. The lap shear strength of the adhesives was then measured under tensile stress at a rate of 1 mm/min using an Instron mechanical tester. The ultimate stress was reported as shear strength of the bioadhesives (n≥5).

In vitro burst pressure: The burst pressure of the bioadhesives, Evicel®, and CoSEAL™ were determined using a modified ASTM (F2392-04) test as described previously (Annabi, N. et al., Biomaterials, 2017, 139:229-243). A piece of porcine intestine was fixed between the stainless-steel annuli of a custom designed burst pressure set up. A 2 mm defect was then created on the center of the tissue. Next, 30 μl precursor solution was applied to the defect site and crosslinked using a dental light curing system. Air pressure was then applied to the sealed tissue and the maximum resistance pressure was recorded as burst pressure (n≥5).

### In Vitro Antimicrobial Properties of Adhesive Hydrogels

P. gingivalis (a clinical isolate A7436 (Papathanasiou, E. et al., Journal of Dental Research, 2016, 95:1018-1025)) was used to evaluate the antimicrobial properties of GelAMP bioadhesives. P. gingivalis was grown on 5% sheep's blood agar plates supplemented with hemin and vitamin K (H & K) in an anaerobic system (5% H₂, 15% CO₂, 80% N₂) at 37° C. for 7 days. The bacteria colonies were then transferred to Wilkins-Chalgren Anaerobe Broth (Oxoid™) media to prepare a 10⁸ CFU/ml bacterial solution. For antimicrobial tests, 1 ml of a 10⁸ CFU/ml bacteria solution was seeded on cylindrical hydrogels with and without AMP (0 and 0.2% (w/v) or 1.34 mM) in 24-well plates. After 72 h anaerobic incubation, the samples were removed from the media and washed gently with DPBS 3 times. Next, each sample was placed in 1 ml DPBS and vortexed for 15 min to release bacteria from within the scaffold. The solutions were then logarithmically diluted to 10⁻¹, 10⁻³, and 10⁻⁴ dilutions. A 30 μl volume of each dilution was then seeded on sheep's blood agar plates with H & K and incubated for 5 days. The number of colonies was counted and reported for each sample (n=4). For SEM imaging, hydrogels were removed from the media and washed 3 times with DPBS. The samples were then fixed in 2.5% (v/v) glutaraldehyde (Sigma-Aldrich) and 4% (v/v) paraformaldehyde (Sigma-Aldrich) in DPBS for 30 min. After fixation, the samples were gently washed 3 times with DPBS and dehydrated using a serially diluted ethanol solution in water (30%, 50%, 70%, 90% and 100% (v/v)). The samples were then dried using a critical point dryer. Lastly, the samples were mounted on aluminum SEM stubs, sputter coated with 6 nm of gold/palladium, and imaged by a Hitachi S-4800 SEM (n=3).

### In Vitro Cell Studies

Cell lines: Bone marrow mouse stromal cells (W-20-17) were cultured at 37° C. and 5% CO₂ in Minimum Essential Medium (MEM) Alpha media (Gibco), containing 10% (v/v) fetal bovine serum (FBS) and 1% (v/v) penicillin/streptomycin (Gibco).

2D cell seeding on adhesive hydrogels: Hydrogels were formed by pipetting 7 μl of precursor solution between a 3-(trimethoxysilyl) propyl methacrylate (TMSPMA, Sigma-Aldrich) coated glass slide and a glass coverslip separated with a 100 μm spacer. Bioadhesive hydrogels were photocrosslinked using visible light for 60 sec. The hydrogels were seeded with W-20-17 cells (5×10⁶ cells/ml) and kept at 37° C., 5% CO₂ for 5 days.

3D cell encapsulation within the engineered hydrogels: For 3D cell encapsulation, a cell suspension of W-20-17 cells (5×10⁶ cells/ml) was prepared by trypsinization and re-suspension in MEM alpha medium. The cell suspension was centrifuged to form a cell pellet and the media was discarded. A hydrogel precursor containing 7% bioadhesive was prepared in culture media containing TEA/VC/Eosin Y and mixed with the cell pellet. Hydrogels were formed by pipetting 7 μl of the precursor solution between a TMSPMA-coated glass slide and a glass coverslip separated with a 100 μm spacer, and photocrosslinking upon exposure to visible light for 60 sec. Lastly, the glass slides with the encapsulated W-20-17 cells were placed in 24 well plates and incubated in MEM alpha at 37° C. and 5% CO₂.

Cell viability, proliferation, and spreading: A calcein AM/ethidium homodimer-1 live/dead kit (Invitrogen) was used to evaluate cell viability as described previously (Annabi, N. et al., Science Translational Medicine, 2017, 9: eaai7466). Cell proliferation and metabolic activity was determined using a commercial PrestoBlue assay (Fisher) on days 0, 1, 3, and 5 as described previously (Shirzaei, E. et al., ACS Biomaterials Science & Engineering, 2018, 4:2528-2540). Briefly, PrestoBlue solution was added to the culture media (10% v/v) with the cell seeded scaffolds and incubated for 45 min at 37° C. Next, the fluorescence intensity of the incubated solutions was evaluated using a BioTek plate reader (excitation: 535-560 nm; emission: 590-615 nm). The relative fluorescence intensity was reported at different time points. Cell spreading on 2D and 3D cultures was evaluated via fluorescent staining of F-actin microfilaments and cell nuclei (Shirzaei, E. et al., ACS Biomaterials Science & Engineering, 2018, 4:2528-2540). Briefly, cell seeded hydrogels were fixed in 4% (v/v) paraformaldehyde (Sigma-Aldrich) for 20 min, and washed three times with DPBS at days 1, 3, and 5 of culture. Samples were then permeabilized in 0.1% (w/v) Triton X-100 (Sigma) in DPBS for 20 min. Next, samples were incubated with Alexa-fluor 488-labeled rhodamine-phalloidin (2.5% (v/v) in 0.1% BSA, Invitrogen) for 45 min. Samples were washed three times with DPBS, and stained again with 1 μl/ml DAPI (4′,6-diamidino-2-phenylindole, Sigma-Aldrich) in DPBS for 5 min. Lastly, the cell seeded hydrogels were washed three times with DPBS and fluorescent images were acquired using an Axio Observer Z1 inverted microscope.

### Animal Studies

Calvarial bone suture tissue extraction and encapsulation into the gels: All animal experiments were performed according to the Guide for the Care and Use of Laboratory Animals (IACUC approval IS00000535) at Harvard School of Dental Medicine. For all experiments, 7-8 weeks-old wild type house mice (Mus musculus) were used. To obtain the calvarial bone sutures, mice were first euthanized by CO₂ inhalation, followed by cervical dislocation. After decapitation, the head was cleaned using 70% ethanol. A cut was then created through the skin at the base of the skull, using a surgical blade. Next, an incision was made starting at the nose bridge and ending at the base of the skull followed by removal of the skin from the top of the head. The calvaria was then cut and transferred to a petri dish with DPBS. After washing with DPBS, the soft tissues were removed using tweezers and the sutures were isolated using scissors. The isolated tissues were chopped into small fragments of 1-2 mm² and quickly transferred to ice-cold cell culture media prior to use. For encapsulation, the suture fragments were placed on a flat petri dish, in between two spacers (500 μm). Then 70 μl of the bioadhesive precursor was pipetted on the tissue samples and covered by a glass cover slip. The samples were then photocrosslinked for 2 min using a dental curing light. Samples were removed from petri dishes and placed in 12 well tissue culture plates. Next, 2 ml MEM Alpha media, containing 10% (v/v) fetal bovine serum (FBS) and 1% (v/v) penicillin/streptomycin were added to each well and the samples were incubated at 37° C. for up to 30 days. The samples were imaged using a Zeiss Primo Vert inverted microscope, and the cell metabolic activity was evaluated as described before (n≥3).

Mouse calvarial bone defect model: Male and female mice were assigned randomly to all experimental groups. After general anesthesia, 2-mm round defects were made with surgical bur on right and left parietal bone of mice. Next, 10 μl of the precursor solution were injected in the defect sites (7% and 15% (w/v)) and photopolymerized using a dental light curing unit for 1 min. After anatomical wound closure, the animals recovered from anesthesia. At each time point, the animals were euthanized by CO₂ inhalation, followed by cervical dislocation. After euthanasia, calvarial tissues were collected for μCT and histological analysis (n≥3).

### Micro Computed Tomography (μCT) Analysis

After explanation, the tissue samples were fixed in 70% (v/v) ethanol prior to scanning (4° C.). Next, the samples were imaged using a desktop cone-beam μCT, (source voltage: 70 kVp, power: 8 W, exposure time: 300 ms, and voxel size: 10 microns) SCANCO μCT 35 (SCANCO Medical, Switzerland). To determine bone volume and surface area, the images were analyzed using the BoneJ software as described previously (Doube, M. et al., Bone 2010, 47:1076-1079).

### Histological Analysis

After μCT analysis, the tissues were fixed with 4% (v/v) paraformaldehyde in DPBS overnight. Next, the samples were washed three times with DPBS and decalcified in Morse's solution (22.5% (v/v) formic acid, 10% (w/v) sodium citrate) at 4° C. After 4 days, the samples were removed from the solution and washed with DPBS. Next, the samples were embedded in Optimal Cutting Temperature compound (OCT), followed by flash freezing in liquid nitrogen. The embedded samples were sectioned (10 μm) using a Leica CM3050 S cryostat. For H&E staining (Sigma-Aldrich), the samples were stained according to the manufacturer's instructions.

### Statistical Analysis

All data were presented as mean±standard deviation (*p<0.05, **p<0.01, ***p<0.001 and ****p<0.0001). T-test, one-way, or two-way ANOVA followed by Tukey's test were performed using the GraphPad Prism 6.0 Software.

### Results and Discussion

Hydrogel-based bioadhesives hold remarkable potential for soft and hard tissue engineering applications due to their tunable composition and physical properties. The precise control over the microarchitecture, mechanical properties and degradation rate of hydrogels, make them great alternatives for the controlled delivery of a variety of therapeutic agents in vivo. Antimicrobial hydrogel adhesives, based on extracellular matrix (ECM)-derived biopolymers, have been developed for the treatment of chronic non-healing wounds (Annabi, N. et al., Biomaterials, 2017, 139:229-243) and orthopedic applications (Cheng, H. et al., ACS Appl. Mater. Interfaces, 2017, 9:11428-11439). In the field of regenerative dentistry, previous studies have reported the engineering of hydrogels based on the combination of alginate with the soluble and insoluble fractions of the dentin matrix (Athirasala, A. et al., Biofabrication, 2018, 10: 024101). More recently, cell-laden gelatin-based hydrogels have been developed that could be photopolymerized using dental curing lights (Monteiro, N. et al., Dental Materials, 2018, 34:389-399). However, the development of antimicrobial hydrogels that can strongly adhere to hard and soft oral surfaces for the treatment of PIDs has not been reported. The engineering of therapeutic approaches that could enable compartmentalized tissue healing by sealing the affected area and thus preventing migration of bacteria and other unwanted cells to the healing site (Gottlow, J. et al., J. Clin. Periodontol., 1984, 11:494-503; Nyman, S., J. Clin. Periodontol., 1991, 18:494-498) may improve clinical outcome in patients with PI. Therefore, the goal of the present invention is to develop osteoinductive and antimicrobial adhesives that: 1) can be rapidly photocrosslinked in situ using dental curing lights, 2) are able to strongly adhere to soft/hard oral tissues, as well as implant surfaces in the presence of blood and saliva, 3) exhibit potent antimicrobial activity, and 4) can induce bone regeneration without the need for exogenous growth factors. The unmet need to develop antimicrobial and regenerative strategies for treatment of PIDs led to the antimicrobial and osteoinductive hydrogel adhesives of the present invention that, by adhering to both hard (titanium implants) and soft tissue (gingiva) surfaces, will allow compartmentalized tissue healing and foster tissue regeneration.

Herein, the development of a visible light-crosslinkable antimicrobial hydrogel adhesive for the treatment of PIDs is described. The hydrogel precursors are delivered in a minimally invasive manner to the PI site and are photocrosslinked in situ using visible light. This bioadhesive was engineered through the incorporation of a cationic AMP (Tet213) into a photocrosslinkable gelatin methacryloyl hydrogels to form GelAMP bioadhesives. The presence of AMP Tet213 renders the resulting hydrogel adhesives antimicrobial (Ageitos, J. M. et al., Biochem. Pharmacol., 2016, 133:117-138). AMPs are small cationic and hydrophobic peptides that possess high antibacterial activity at very low concentrations (Cheng, H. et al., ACS Appl. Mater. Interfaces, 2017, 9:11428-11439; Annabi, N. et al., Biomaterials, 2017, 139:229-243). In addition, AMPS do not readily lead to the selection of resistant mutants, which makes them ideal candidates to prevent bacterial growth in biomedical implants via local delivery (Kazemzadeh-Narbat, M. et al., Biomaterials, 2010, 31:9519-9526). The use of AMP-loaded hydrogels represents a novel approach to treat bacterial infections around dental implants, without the need for conventional antibiotics.

The physical and the adhesive properties of the resulting bioadhesives were studied in vitro. Additionally, the antimicrobial properties of the bioadhesives against Porphyromonas gingivalis (P. gingivalis), a Gram-negative bacterium that is involved in the pathogenesis of PIDs were evaluated. The cytocompatibility of the bioadhesives was also evaluated in vitro via two-dimensional (2D) surface seeding and three-dimensional (3D) encapsulation of W-20-17 murine fibroblasts. Lastly, the ability of the bioadhesives to support bone regeneration in vivo was studied using a calvarial defect model in mice. The engineered antimicrobial bioadhesives could constitute an effective approach to prevent bacterial growth, while also supporting tissue regeneration for the treatment of PIDs.

### Physical Properties of the Bioadhesives

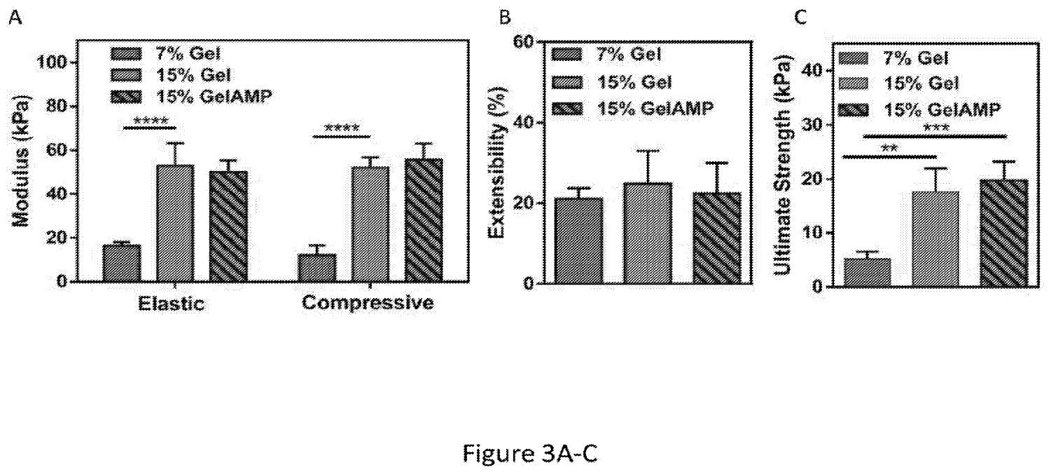

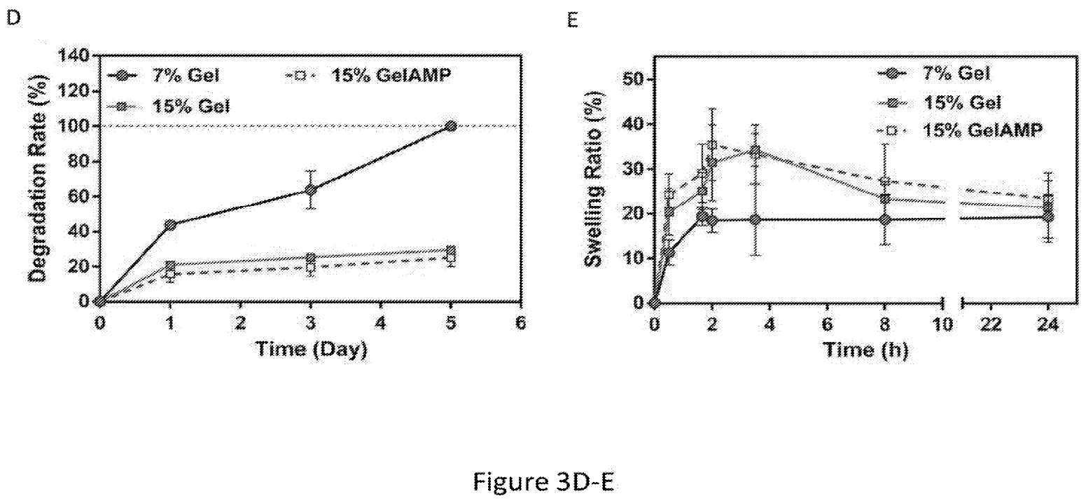

To evaluate the physical properties of the bioadhesives, hydrogel formulations were synthesized based on two different concentrations of bioadhesive (7 and 15% (w/v)) with and without incorporation of AMP. The results showed that 15% (w/v) bioadhesive hydrogels exhibited a 4.3-fold and 3.2-fold increase in the compressive and elastic moduli, respectively, when compared to 7% (w/v) hydrogels (FIG. 3A). In addition, the extensibility of the bioadhesives did not change by changing the concentration of bioadhesive from 7% to 15% (w/v) or by the addition of AMP (FIG. 3B). However, the ultimate tensile strength of hydrogels increased from 5.2±1.3 kPa to 19.8±3.5 kPa as the bioadhesive concentration was increased from 7% to 15% (w/v) (FIG. 3C). The results also showed that the addition of AMP did not alter the mechanical properties of the bioadhesives, which could be due to the low concentration and the small size of the AMP (Annabi, N. et al., Biomaterials, 2017, 139:229-243).

Next the in vitro stability of the bioadhesives was examined by incubating the bioadhesives in collagenase type II solution in DPBS (20 μg/ml) for 5 days. Bioadhesives with 7% (w/v) concentration resulted in significantly accelerated degradation as compared to bioadhesives with 15% (w/v) concentration. In particular, the 7% (w/v) bioadhesive showed 100.0% degradation by day 5 post-incubation, while 29.4±2.2% of the hydrogel with 15% (w/v) concentration was degraded during the same time (FIG. 3D). In addition, there was no significant difference in the degradation of bioadhesive hydrogels with or without AMP (FIG. 3D).

The water uptake capacity of the hydrogels was determined by calculating the swelling ratios of the bioadhesives at different concentrations and time points. For this measurement, the swelled weights of the samples after incubation at 37° C. in DPBS was divided by their corresponding dry weights. As is shown in FIG. 3E, the swelling ratios of the hydrogels decreased by increasing bioadhesive concentrations. However, the swelling ratios barely changed after 10 h of incubation, indicating that the equilibrium states were achieved at this time point. In addition, the incorporation of AMP did not alter the degradation rate and the swellability of the bioadhesives (FIGS. 3E-F). Overall, bioadhesives with 15% (w/v) concentration showed higher mechanical stiffness and slower degradation rates as compared to 7% (w/v) hydrogels.

Previous studies have also studied the effect of physical properties and microstructural features of hydrogel scaffolds on the regeneration and repair of target tissues (Annabi, N. et al., Biomaterials, 2017, 139:229-243; Monteiro, N. et al, Dental Materials, 2018, 34:389-399; Huebsch, N. et al., Nature Materials, 2015, 14:1269-1277). An ideal bioadhesive used in the setting of the oral cavity should be elastic and flexible, as well as sufficiently strong to withstand breakage due to the intrinsic dynamism of the oral tissues (Peh, K. K. et al., J. Pharm. Pharm. Sci., 1999, 2:53-61). For this purpose, the water uptake capacity of the bioadhesives should be finely tuned to prevent excessive swelling, which could lead to patient discomfort and detachment from the wet and highly motile oral tissues. Furthermore, fast degradation of the adhesive could compromise adequate retention and greatly limit their clinical efficacy (Annabi, N. et al., Biomaterials, 2017, 139:229-243). The present results showed that, in addition to the higher modulus (FIG. 3A), and ultimate strength (FIG. 3C) of the 15% (w/v) bioadhesives, comparatively higher structural stability was seen in vitro. This was demonstrated by the slower degradation rates (FIG. 3D) and similar swelling equilibrium states upon incubation in DPBS (FIG. 3E) when compared to 7% (w/v) bioadhesives. Next, the adhesive properties of the hydrogels to soft physiological tissues and hard implant surfaces was evaluated.

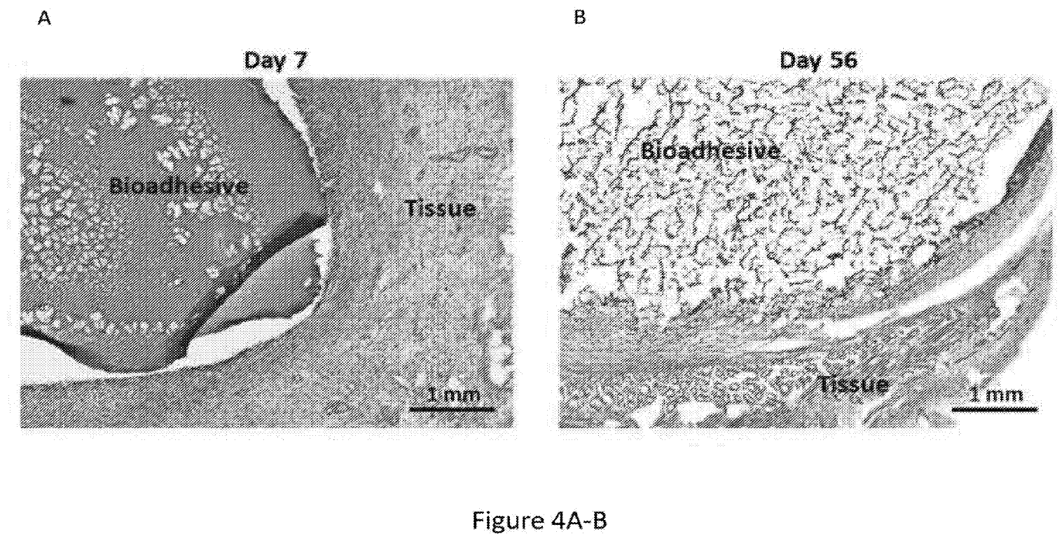

The in vivo biodegradation of GelAMP bioadhesive was also confirmed in a rat subcutaneous implantation model. Accordingly, hematoxylin and eosin (H&E)) analysis of the explanted samples revealed a significant deformation and biodegradation of hydrogels after 56 days of implantation when compared to day 7 (FIG. 4A-B). This can be mainly due to the enzymatic hydrolysis of the gelatin backbone (Shirzaei Sani, E. et al., ACS Biomaterials Science & Engineering, 2018, 4: 2528-2540).

### In Vitro and Ex Vivo Characterization of the Adhesive Properties

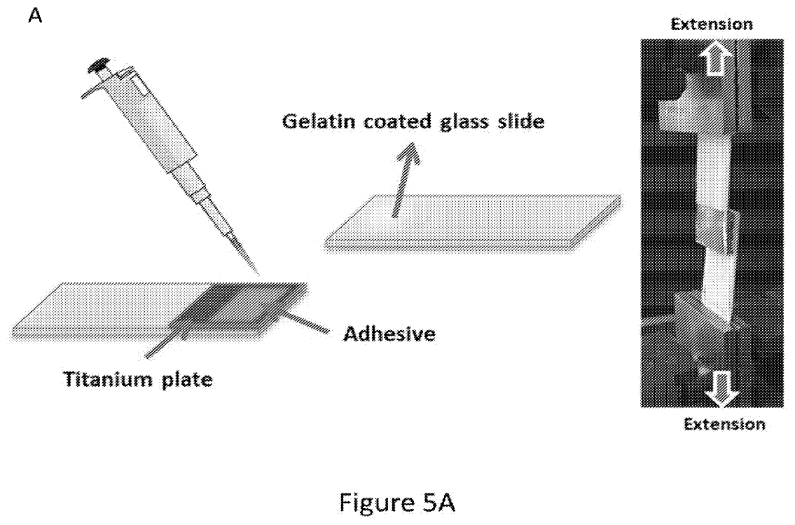

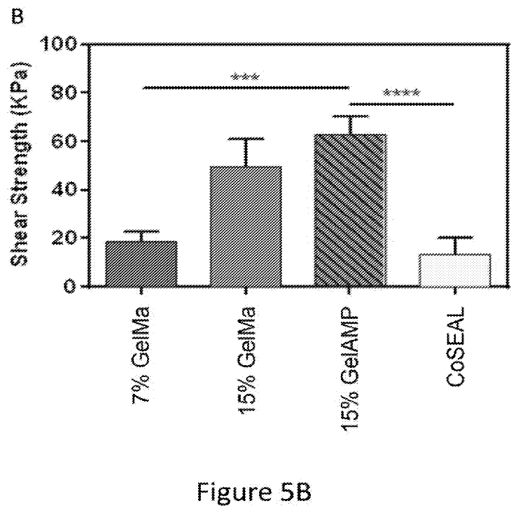

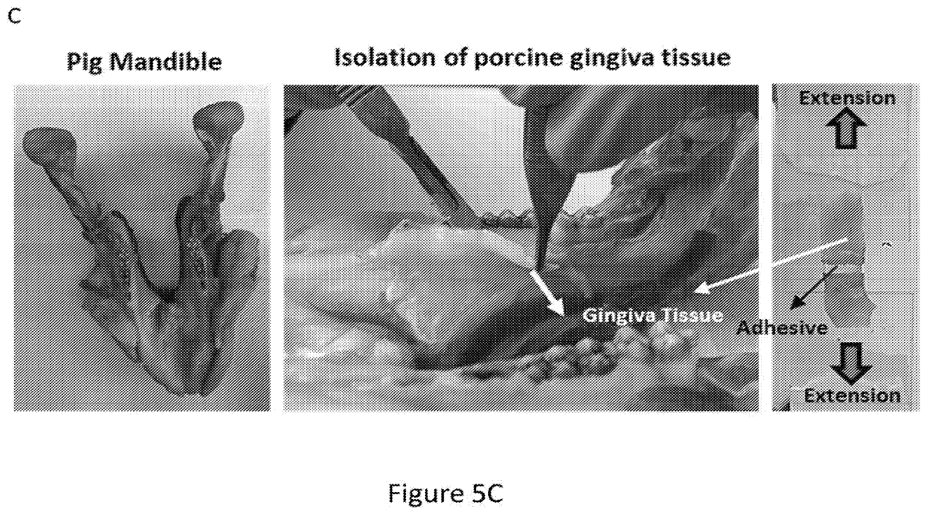

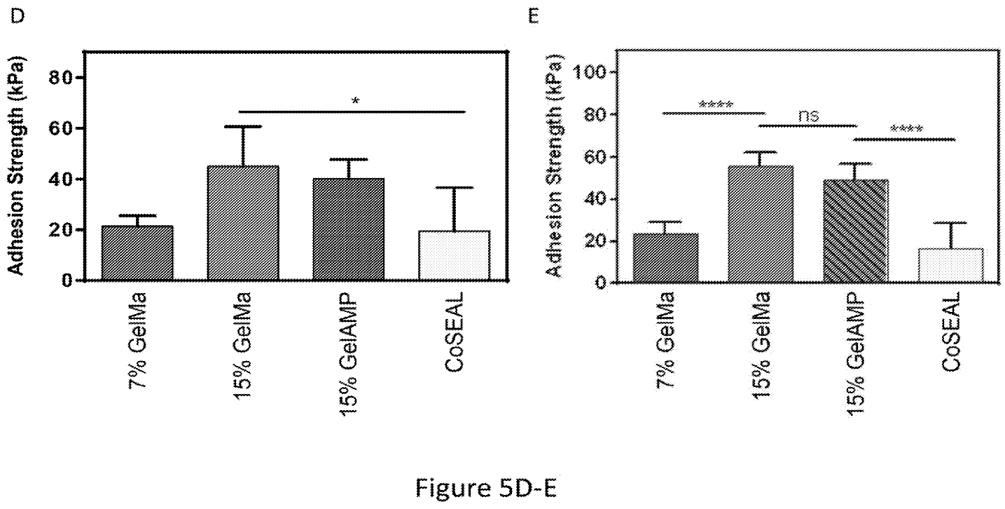

The strong retention and adhesion of biomaterials to both the native tissue and the implant surface is a critical factor to promote periodontal tissue repair and regeneration (Nasajpour, A. et al., Advanced Functional Materials, 2018, 28:1703437). Moreover, the designed bioadhesive must withstand the shear and the pressure exerted by the underlying tissues and the high motility of the oral tissues. To evaluate these parameters, standard in vitro adhesion tests were performed including wound closure (ASTM F2458-05), lap shear (ASTM F2255-05), and burst pressure (ASTM F2392-04) to assess the adhesiveness of the hydrogels to physiological tissues and titanium surfaces (FIG. 5A). Similar tests were also performed using a commercially available sealant, CoSEAL™, as a control. Wound closure tests were performed to measure the adhesive strength of the bioadhesives to soft tissues including porcine gingiva (FIGS. 5C-D) and porcine skin (Figure SE). The results of the wound closure tests revealed that the adhesive strength of the hydrogel to gingiva increased from 23.5±5.4 kPa to 55.3±6.7 kPa, by increasing the hydrogel concentration from 7 to 15% (w/v) (FIG. 5D). Similarly, the adhesive strength of the bioadhesives to porcine skin was increased 2.1-fold by increasing the total polymer concentration from 7 to 15% (w/v) (FIG. 5E). Moreover, the presence of AMP did not alter the adhesion strength of the hydrogels for both porcine gingiva and skin (FIG. 4D, FIG. 5E). Lastly, the adhesive strength of the 15% (w/v) bioadhesive was significantly higher than that of CoSEAL™, with a 3.3-fold difference for gingiva tissue and a 1.7-fold difference for skin tissue (FIG. 5D and FIG. 5E).

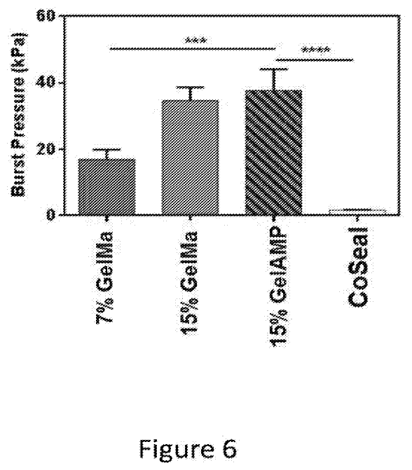

Similar to the wound closure tests, 15% (w/v) bioadhesives, with and without AMP, showed significantly higher lap shear strength to titanium surface as compared to CoSEAL™ (i.e., 3.7 and 4.6-fold difference, respectively) (FIG. 5B). However, the lap shear strength did not significantly change for 15% (w/v) bioadhesives with and without AMP (FIG. 5B). In contrast, the burst pressure of the bioadhesives was increased from 17.0±2.9 kPa at 7% (w/v) to 34.6±4.0 kPa at 15% (w/v) final polymer concentration. Furthermore, the highest burst pressure was observed for 15% (w/v) hydrogels (37.7±6.5 kPa), which was significantly higher than that of CoSEAL™ (1.7±0.1 kPa) (FIG. 6).

Different hydrogel adhesives have been used for sealing, reconnecting tissues, or as implant coatings (Cheng, H. et al., ACS Appl. Mater. Interfaces, 2017, 9:11428-11439; Nasajpour, A. et al., Advanced Functional Materials, 2018, 28:1703437). However, poor mechanical properties and adhesion to wet tissues have limited the implementation of known hydrogel adhesives in the clinic. Moreover, the majority of the commercially available dental adhesives are based on polymethyl methacrylate (PMMA) or acrylic based resins, which are mainly used as fillers for dentin cavities. Although these types of adhesives have shown strong adhesion and binding to the oral surfaces and tissues (i.e., gingiva and pulpal walls), their potential as a platform for the treatment of PIDs is limited (Purk, J. H. et al., J. Am. Dent. Assoc., 2006, 137:1414-1418; Sofan, E. et al., Ann. Stomatol., 2017, 8:1-17). This is mainly due to the lack of cell-binding sites, and poor tissue biointegration, which ultimately limit the regenerative capacity of these resins (Sofan, E. et al., Ann. Stomatol., 2017, 8:1-17). In contrast, the inventive visible light curable bioadhesives are able to bind strongly to both hard (titanium) and soft (gingiva) surfaces and withstand high shear stress and pressure. In addition, gelatin-based bioadhesives have been shown to strongly adhere to wet and dynamic tissues such as the lungs (Assmann, A. et al., Biomaterials, 2017, 140:115-127). Therefore, these bioadhesives could be used to effectively adhere to periodontal tissues in the presence of blood and saliva, as well as under palatal pressure and during mastication. Moreover, due to the high regenerative capacity of ECM-derived biopolymers, gelatin-based bioadhesives could constitute a suitable alternative for the treatment of PIDs (Annabi, N. et al., Biomaterials, 2017, 139:229-243).

### In Vitro Evaluation of the Antimicrobial Properties of the Bioadhesives

AMPs are comprised of short sequences of cationic amino acids, which have been shown to possess broad spectrum bactericidal activity against G (+/−) bacteria (Annabi, N. et al., Biomaterials, 2017, 139:229-243; Kazemzadeh-Narbat, M. et al., Biomaterials, 2010, 31:9519-9526). AMPs bind to the negatively charged outer leaflet of bacterial cell membranes, which leads to changes in bacterial surface electrostatics, increased membrane permeabilization, and cell lysis (Annabi, N. et al., Biomaterials, 2017, 139:229-243).

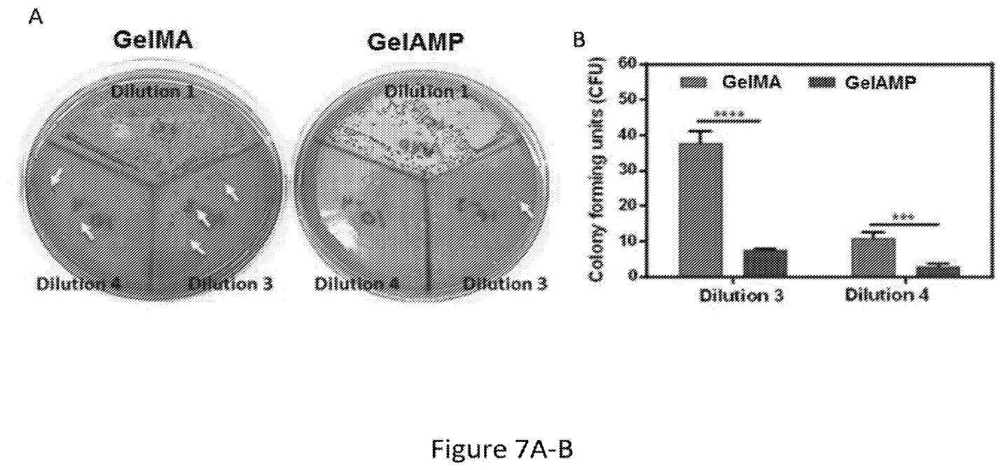

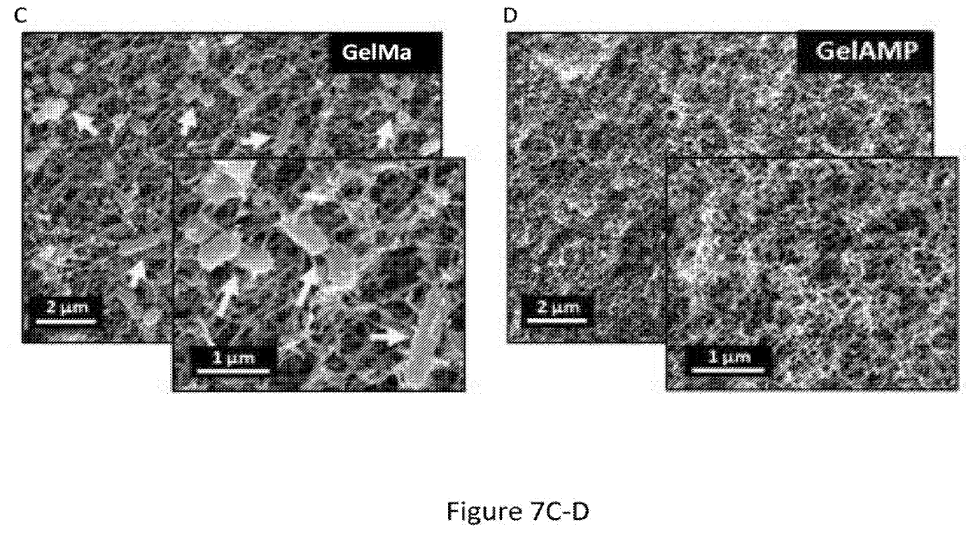

The inventive bioadhesive, GelAMP, was synthesized. GelAMP is a dental light curable bioadhesive hydrogel having antimicrobial properties due to the incorporation of AMP. Previously, it has been shown that AMP tet213 at very low concentrations is effective against both G (+/−) bacteria (Annabi, N. et al., Biomaterials, 2017, 139:229-243). An optimized concentration of AMP was used in this work (0.2% (w/v)), based on previous studies (Annabi, N. et al., Biomaterials, 2017, 139:229-243). First, the antimicrobial activity of the resulting bioadhesive adhesives against P. gingivalis was evaluated using a standard colony forming units (CFU) assay and direct visualization of the bacteria-laden hydrogels via scanning electron microscope (SEM) (FIG. 7A-D). The CFU assay showed that the number of P. gingivalis colonies in the 3-logarithmic dilution decreased from 37.7±3.5 at 0.0% (w/v) AMP, to 10.6±1.9 at 0.2% (w/v) AMP (FIG. 7A-B). A similar response was also observed for the 4-logarithmic dilution, which further confirmed the bactericidal properties of the engineered antimicrobial GelAMP bioadhesives, when compared to pristine hydrogels as controls (FIG. 6B). SEM micrographs also showed that the hydrogels without AMP exhibited significant bacterial infiltration and colonization throughout the polymer network (FIG. 7C). In contrast, GelAMP containing 0.2% (w/v) AMP, showed high antimicrobial activity as demonstrated by the complete absence of bacterial clusters on both surface and cross sections of the bioadhesives (FIG. 7D).

A variety of AMPs such as defensins and cathelicidins are normally found in the oral cavity, particularly in the gingival crevicular fluid and in salivary secretions and constitute the first line of defense against bacterial infection (Khurshid, Z. et al., Curr. Pharm. Des., 2018, 24:1130-1137). Moreover, AMPs do not trigger resistance mechanisms and play a key role in the regulation of microbial homeostasis and the progression of gingival and periodontal diseases (Mallapragada, S. et al., J. Indian Soc. Periodontol., 2017, 21:434-438). Because of this, previous studies have explored the use of AMPs as active coatings for dental implants and other therapeutic strategies aimed at the prevention of bacterial infection (Shi, J. et al., Scientific Reports, 2015, 5:16336; Holmberg, K. V. et al., Acta Biomaterialia, 2013, 9:8224-8231; Yoshinari, M. et al., Biofouling, 2010, 26:103-110). However, AMPs are highly susceptible to proteolytic degradation by proteases secreted by bacteria and host cells and thus, efficient in vivo delivery of AMPs to the site of infection remains challenging. Thus, the engineered inventive bioadhesives could be used to protect AMPs from environmental degradation and to deliver physiologically relevant concentrations of AMPs for controlled periods of time.

### Cell Studies of the Bioadhesives

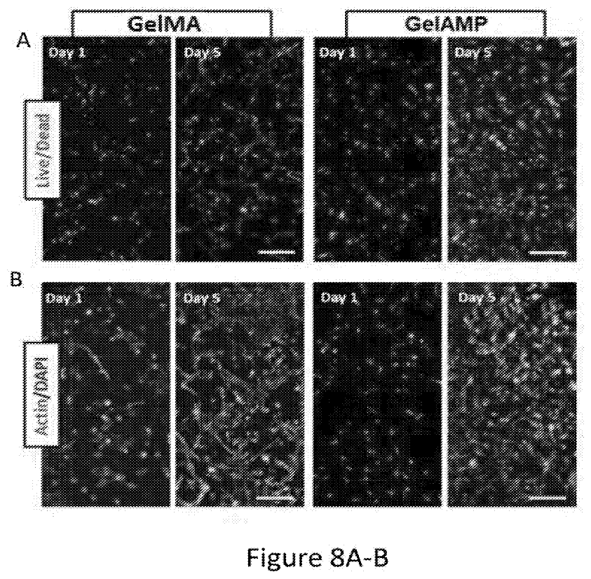

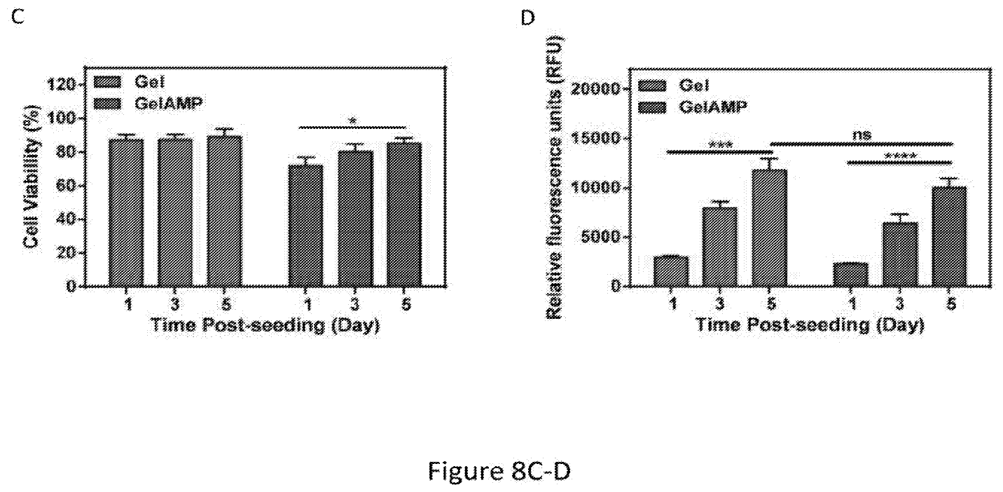

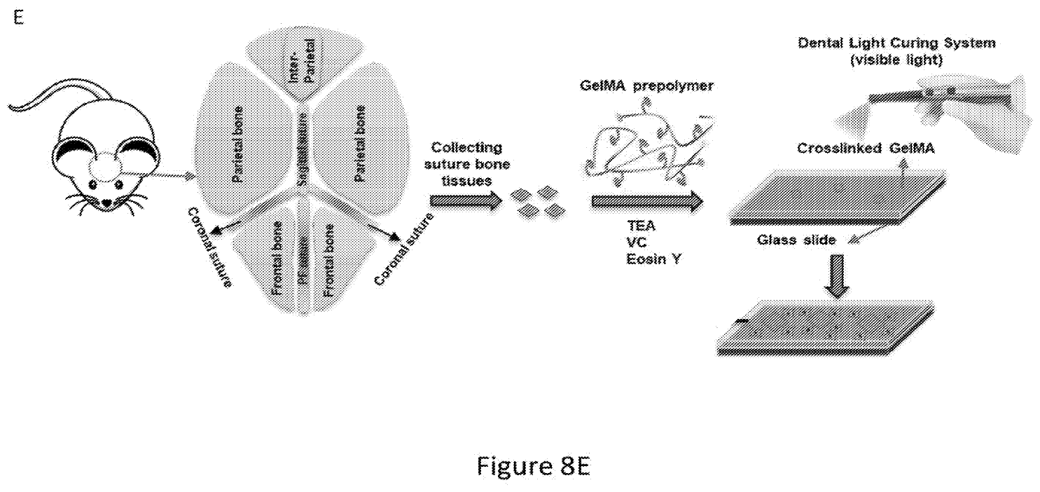

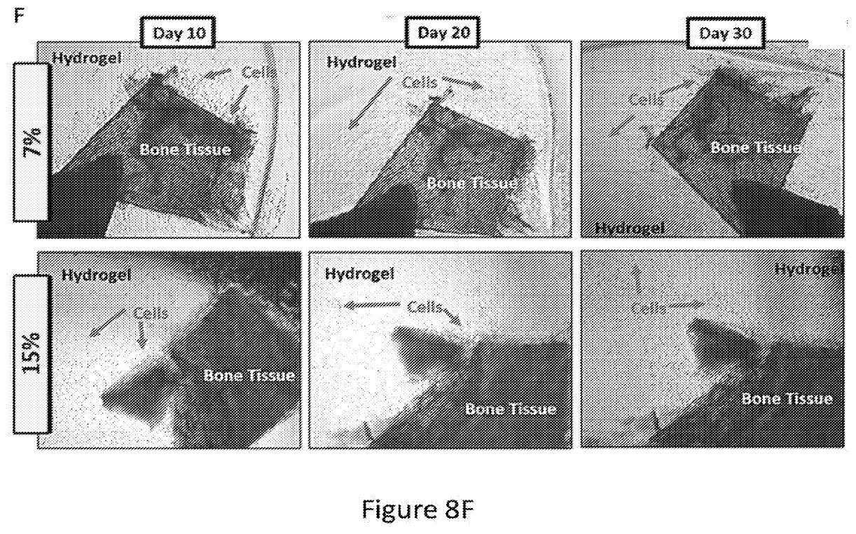

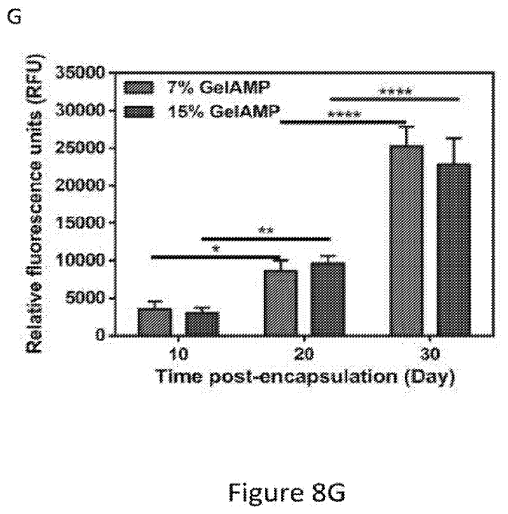

An ideal bioadhesive not only must be cytocompatible but should also allow the attachment and proliferation of cells within the 3D microstructure to support biointegration and healing. Herein, the ability of the engineered bioadhesives to support the attachment and proliferation of migratory cells from the bone stroma via 3D encapsulation of bone marrow stromal cells was assessed (FIG. 8A-G). In addition, the ability of the bioadhesives to support the growth and proliferation of migratory stromal cells via 3D encapsulation of freshly isolated calvarial bone sutures was evaluated.

In vitro cytocompatibility and proliferation of 3D encapsulated cells within Bioadhesive hydrogels: First, the viability, metabolic activity, and spreading of bone marrow mouse stromal cells (W-20-17 (Thies, R. S. et al., Endocrinology, 1992, 130:1318-1324)) encapsulated within the adhesives was evaluated using a live/dead and PrestoBlue assays, and F-Actin/DAPI staining, respectively. The results showed that cells encapsulated within the bioadhesives with and without AMP exhibited >90% viability after 1, 3, and 5 days of culture (FIGS. 8A and 8C). In addition, the incorporation of AMP, did not affect the viability of the encapsulated cells (FIGS. 8A and 8C). Moreover, F-Actin/DAPI staining revealed that W-20-17 cells could attach and proliferate throughout the 3D network for GelMA and GelAMP adhesives, up to 5 days of culture (FIG. 8B). Furthermore, the metabolic activity of cells in GelAMP hydrogels increased consistently from 2273±66 RFUs at day 1 to 10041±938 RFUs at day 5 of culture (FIG. 8D). In addition, there were no statistically significant differences between the metabolic activity of cells seeded on GelAMP and GelMA adhesives (FIG. 8D).

3D encapsulation of calvarial bone suture explants within bioadhesives: The freshly isolated calvarial bone sutures were encapsulated in both 7 and 15% (w/v) hydrogels to evaluate the ability of the bioadhesives to support the proliferation and migration of stromal cells (FIG. 8E). During the first week of encapsulation, no significant cell migration was observed. A week after encapsulation, cell (most likely suture-derived skeletal stem cells (Maruyama, T. et al., Nature Communications, 2016, 7:10526; Wilk, K. et al., Stem Cell Reports, 2017, 8:933-946)) deployment out of the suture was observed, followed by proliferation and migration within the bioadhesive hydrogel (FIG. 8F). The migratory and proliferative behavior of these cells were assessed for up to 30 days post-encapsulation (FIG. 8F). These results showed that the metabolic activity of the encapsulated cells increased consistently for both 7% and 15% (w/v) bioadhesives (FIG. 8F). For instance, the metabolic activity of the cells in 15% GelAMP (w/v) bioadhesives increased from 3016±678 RFUs at day 10, to 22869±3421 RFUs at day 30 post-encapsulation (FIG. 8F). However, no statistical difference between metabolic activity of the cells seeded within the 7% and 15% (w/v) bioadhesive hydrogels was observed (FIG. 8F).

The results also indicated that the antimicrobial bioadhesives did not elicit any cytotoxic response and could effectively support the growth of both W-20-17 and suture-derive skeletal stem cells in vitro. Previous studies have reported the development of different types of antimicrobial hydrogels based on the incorporation of metal or metal oxide nanoparticles (Annabi, N. et al., Biomaterials, 2017, 139:229-243; Mohandas, A. et al., Int. J. Nanomedicine, 2015, 10:53-66; Wahid, F. et al., Polymers, 2017, 9:636). However, the negative effect of metal oxide nanoparticles on cell viability greatly limit their application for the clinical management of PIDs (Wahid, F. et al., Polymers, 2017, 9:636). In contrast, the present results demonstrated that the cells could infiltrate and spread throughout our antimicrobial bioadhesives, while also remaining proliferative and metabolically active.

Taken together, these results demonstrated that the inventive bioadhesives could be used to form an adhesive and antimicrobial barrier that prevents bacterial growth and supports the proliferation of bone-competent cells in vitro. The ability of the bioadhesives to eradicate or prevent infection at the implant site could not only be relevant to disinfect the affected area, but also to reduce inflammatory responses triggered by sustained microbial colonization. Moreover, the establishment of a cell-supportive microenvironment could promote the regeneration of the affected bone by endogenous progenitor cells that migrate into the wound site. Therefore, the ability of the bioadhesives to support bone regeneration in vivo using a calvarial defect model in mice was evaluated.

### In Situ Application and In Vivo Evaluation of Bioadhesive Hydrogels

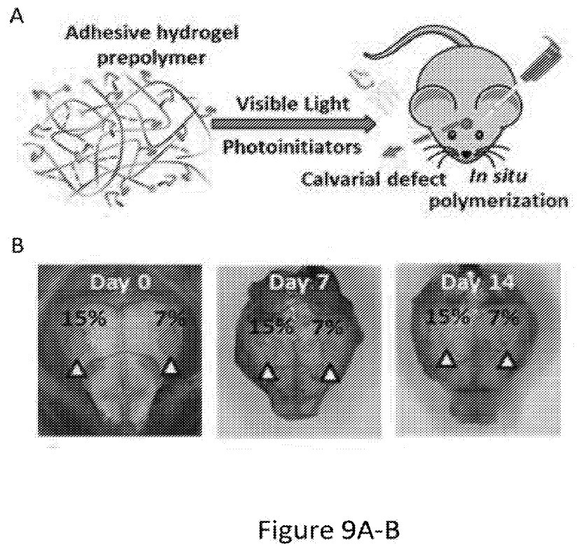

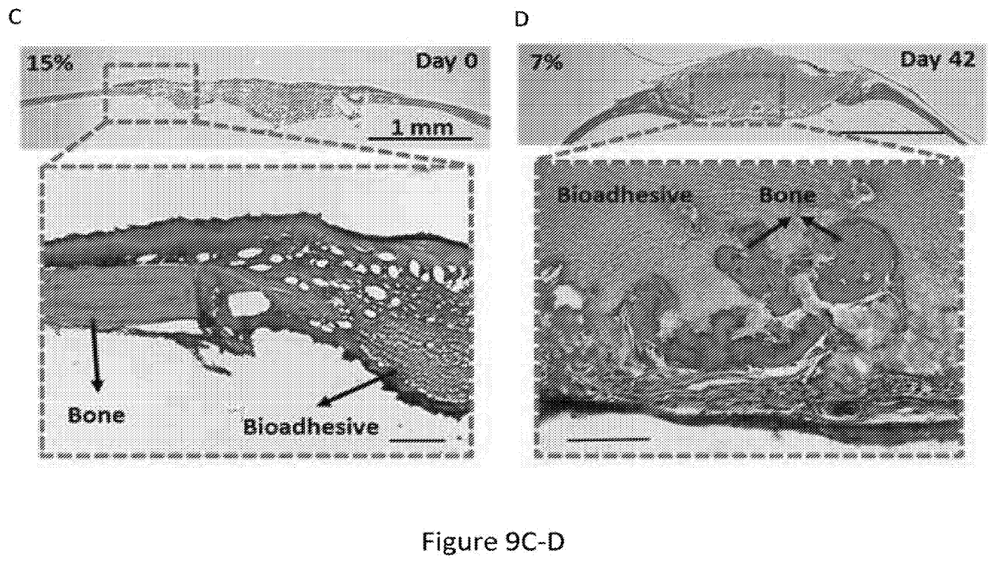

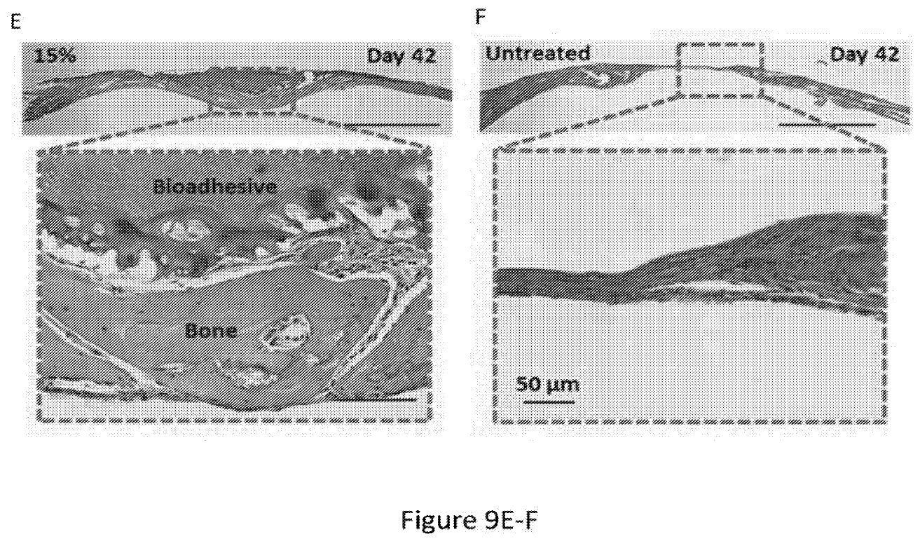

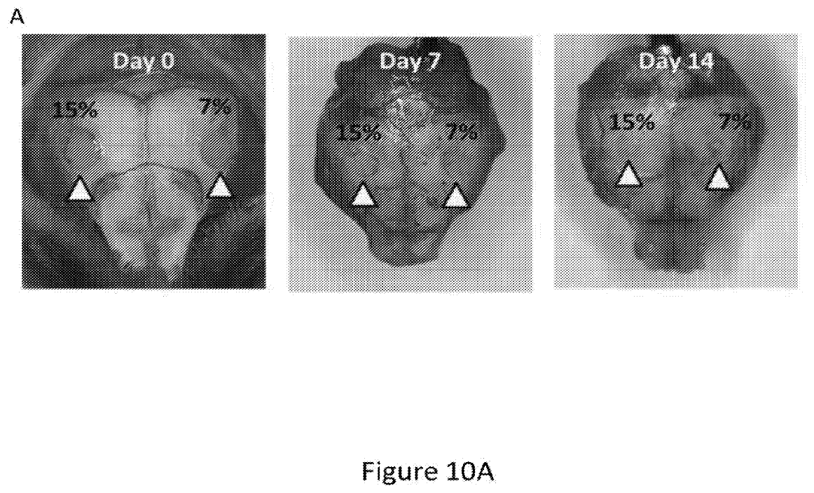

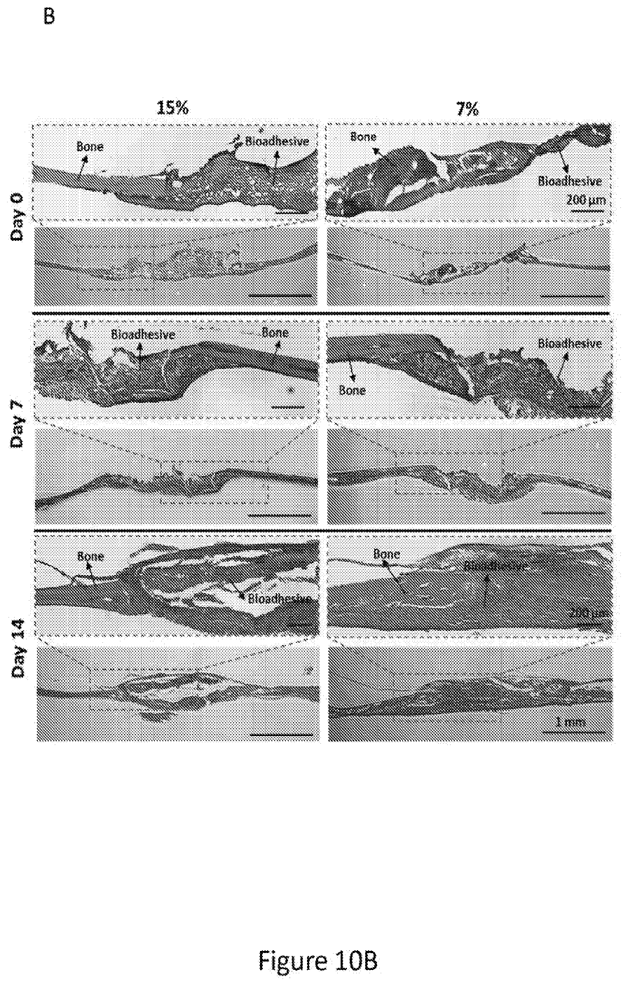

The ability of the hydrogels to be delivered and formed in situ and to remain firmly attached to the wound area without the risk of displacement during the healing process was investigated. For these studies, critically sized defects in mice calvaria were created using dental drills. The bioadhesive precursor solutions (7% and 15% (w/v)) were directly injected into the bone defects and photopolymerized using a commercial dental light curing unit (FIG. 8A). The results showed that the bioadhesives could remain at the site of application without any sign of displacement after 7 and 14 days of implantation (FIG. 9B). In addition, histological assessment (hematoxylin and eosin (H&E)) showed the complete sealing of the defect and a strong coherence between the biopolymer and the native bone following application (FIG. 9C). Moreover, the H&E images also revealed that bioadhesives with both formulations (7, and 15% (w/v)) could remain attached to the wound site up to 42 days after application (FIG. 9D-E). At earlier time points (14 days post application), the formation of new autologous bone could be observed near the margin of the original defect (FIG. 10A-B). Calvarial defects in untreated control animals showed limited new bone formation at day 42 post application (FIG. 9F). In contrast, histological staining revealed the formation of new bone for both 7% and 15% (w/v) bioadhesives (FIG. 9D-E). Furthermore, the area covered by the newly formed bone was significantly larger for defects treated with 15% (w/v) hydrogels as compared to 7% (w/v) hydrogels (FIG. 10A-B). This observation could be explained in part due to the increased structural integrity of bioadhesives with higher polymer concentration, which provided a more structurally stable scaffold to support bone regeneration and the ingrowth of the adjacent connective tissues (FIG. 9E). These observations provided qualitative evidence that was indicative of the formation of new bone and the subsequent repair of the defect.

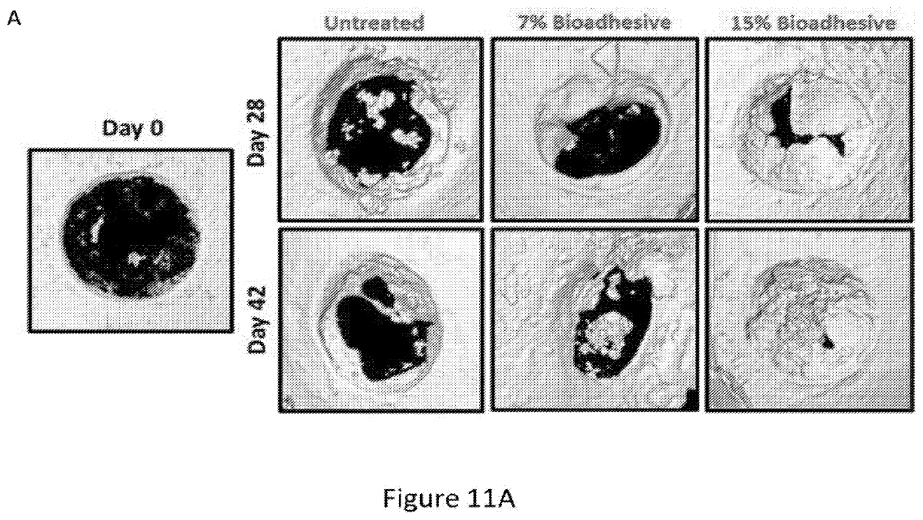

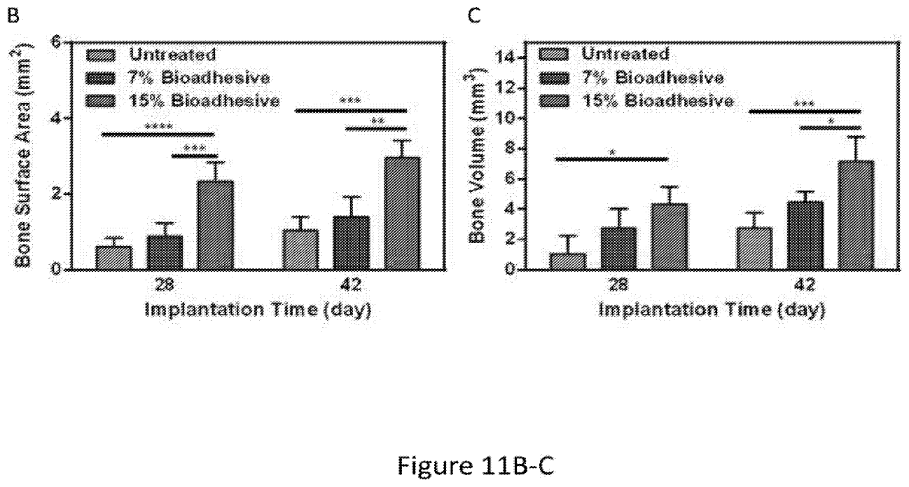

To perform a quantitative evaluation of new bone formation, micro-computed tomography (μCT) was performed on untreated defects, as well as defects treated with bioadhesive synthesized using 7 and 15% (w/v) polymer concentrations at days 0, 28, and 42 post-procedure (FIG. 11A-B). The results showed that the untreated defects exhibited limited evidence of bone forming up to 28- and 42 days post-procedure, with little decrease in the extension of the critical size (FIG. 11A). At day 28, the defects treated with the 15% (w/v) hydrogels showed significantly higher bone formation than 7% (w/v) hydrogels and the untreated controls. At day 42, a significant amount of new bone was observed for defects treated with 15% (w/v) hydrogels (FIG. 11A). In addition, on days 28 and 42, the bone surface area (BS) and the bone volume (BV) for 15% (w/v) hydrogels were shown to be significantly higher than that of untreated and 7% (w/v) groups (FIG. 11B-C). For instance, at day 42, the BS for 15% (w/v) hydrogels corresponded to 2.96±0.46 mm², which was significantly higher than the untreated controls (i.e., 1.03±0.63 mm²) and 7% (w/v) hydrogels (i.e., 1.40±0.53 mm²) (FIG. 11B). Moreover, the highest BV was observed for 15% (w/v) bioadhesives (i.e., 7.16±1.65 mm³), which was significantly higher than those of untreated (i.e., 2.76±1.03 mm³) and 7% (w/v) bioadhesives (i.e., 4.45±0.72 mm³) (FIG. 11C). Statistical analysis indicated that both the concentration of the biopolymer and the treatment time had a significant effect on BV and BS. For instance, the BS and BV increased 1.27 and 1.66-fold respectively, at 28- and 42 days post-procedure, which was indicative of sustained bone regeneration throughout the experiment (FIG. 11B-C).

The higher degree of bone regeneration observed for 15% (w/v) bioadhesive could be due in part to the direct contribution of the enhanced mechanical properties of hydrogels with higher polymer concentrations (Huebsch, N. et al., Nature Materials, 2015, 14:1269-1277). For instance, Huebsch et al. demonstrated that the contribution of matrix elasticity to new bone formation in vivo is highly correlated with mechanically induced osteogenesis (Huebsch, N. et al., Nature Materials, 2015, 14:1269-1277). Huebsch found that the BV and mineral density obtained for hydrogels with elasticities in the range of 60 kPa was significantly higher than those with 5 kPa or 120 kPa moduli (Huebsch, N. et al., Nature Materials, 2015, 14:1269-1277). In the present study, 15% (w/v) bioadhesives, which exhibited elastic and compressive modulus corresponding to 53.0±10.3 kPa and 52.2±4.7 kPa (FIG. 3A), respectively, could potentially enable mechanically induced osteogenesis and thus, promote the formation of new bone in vivo. However, the clinical efficacy of antimicrobial bioadhesives for the treatment of patients with advanced PI could be limited due to the lack of a bona fide osteoinductive strategy. Although previous groups have reported the development of regenerative bioadhesives, such bioadhesives often rely on the use of growth factors (Patterson, J. et al., Biomaterials, 2010, 31:6772-6781; Gibbs, D. M. et al., Journal of Tissue Engineering and Regenerative Medicine, 2016, 10:187-198), stem cells (Huebsch, N. et al., Nature Materials, 2015, 14:1269-1277; Chamieh, F. et al., Scientific Reports, 2016, 6:38814), and other bioactive molecules (Nguyen, M. K. et al., Acta Biomaterialia, 2018, 75:105-114; Kyllonen, L. et al., Acta Biomaterialia, 2015, 11:412-434). These methods often suffer from clinical limitations and drawbacks (Woo, E. J. Journal of Oral and Maxillofacial Surgery, 2012, 70:765-767; Mesfin, A. et al., The Journal of Bone and Joint Surgery, 2013, 95:1546-1553; Hanisch, O. et al., The International Journal of Oral & Maxillofacial Implants, 1997, 12:604-610). Due to these limitations, future work will introduce a cell/growth factor-free strategy by the incorporation of alternative osteoinductive strategies such as nanosilicates (Xavier, J. R. et al., ACS Nano, 2015, 9:3109-3118; Wang, Y. et al., Colloids Surf. B Biointerfaces, 2018, 172:90-97) into antimicrobial bioadhesives which could constitute an attractive platform for the development of osteoinductive and antimicrobial bioadhesives for the treatment of PIDs.

### Conclusion

The clinical management of PIDs still constitutes a significant challenge for clinicians and researchers in the dentistry field. In the present study, antimicrobial hydrogel bioadhesives were engineered for the treatment of PIDs. The hydrogel precursors could be readily delivered and photocrosslinked in situ using commercial dental curing systems. These bioadhesives exhibited tunable mechanical stiffness and elasticity, and comparatively higher adhesive strength to implant and oral surfaces than commercial adhesives. In addition, the bioadhesives showed high antimicrobial activity in vitro against P. gingivalis, a pathogenic bacterium associated with the onset and progression of PIDs. In vitro and ex vivo studies demonstrated that the bioadhesives were highly cytocompatible and could provide a suitable microenvironment for migratory stromal cells deployed from encapsulated bone sutures. Furthermore, in vivo studies showed that the bioadhesives could promote bone regeneration by supporting the growth of migratory progenitor cells. Taken together, these results demonstrate the remarkable potential of the inventive bioadhesive hydrogels to be used as adhesive, antimicrobial, and cell-supportive barriers that can support tissue healing and bone regeneration in vivo for the treatment of PIDs.

### Example 2. An Osteoinductive and Antimicrobial Bioadhesive Hydrogel for Treatment of Peri-Implantitis and Periodontal Bone Defects (with and without Growth Factors)

The unmet need to develop antimicrobial and regenerative strategies for treatment of PIDs led to the engineering of antimicrobial and osteoinductive hydrogel adhesives that, by adhering to both hard (titanium implants) and soft tissue (gingiva) surfaces will allow compartmentalized tissue healing and foster tissue regeneration. In addition, the inventive bioadhesive hydrogels can be used for other orthopedic applications such as a replacement for bone grafts, coating for implants, etc.

Guided bone regeneration (GBR) techniques, which rely on compartmentalized tissue healing (CTH (Gottlow, J. et al., “New attachment formation as the result of controlled tissue regeneration,” J. Clin. Periodontol., 1984, 11(8):494-503; Nyman, S., “Bone regeneration using the principle of guided tissue regeneration,” J. Clin. Periodontol., 1991, 18(6):494-498), are utilized for re-osseointegration of implants affected by PI. Several biomaterials have been proposed to increase the regenerative efficacy of GBR (Jensen, S. S. et al., “Bone augmentation procedures in localized defects in the alveolar ridge: clinical results with different bone grafts and bone-substitute materials,” Int. J. Oral Maxillofac. Implants, 2009, 24 Suppl:218-236; Mellonig, J. T. et al., “Guided bone regeneration of bone defects associated with implants: an evidence-based outcome assessment,” Int. J. Periodontics Restorative Dent., 1995, 15(2):168-185; Simion, M. et al., “Guided bone regeneration using resorbable and nonresorbable membranes: a comparative histologic study in humans,” Int. J. Oral Maxillofac. Implants, 1996, 11(6):735-742). However, GBR remains technically challenging, with large variability in terms of success rate (Fonseca, R. J. et al., “Revascularization and healing of onlay particulate autologous bone grafts in primates,” J. Oral Surg., 1980, 38(8):572-577; Phillips, J. H. et al., “Fixation effects on membranous and endochondral onlay bone graft revascularization and bone deposition,” Plast. Reconstr. Surg., 1990, 85(6):891-897; Jensen, 0. T. et al., “Vertical guided bone-graft augmentation in a new canine mandibular model.,” Int. J. Oral. Maxillofac. Implants, 1995, 10(3):335-344; Tinti, C. et al., “Vertical ridge augmentation: surgical protocol and retrospective evaluation of 48 consecutively inserted implants,” Int. J. Periodontics Restorative Dent., 1998, 18(5):434-443). Autologous bone grafts are the most effective approach to increase the success rate of GBR (Jensen, S. S. et al., “Bone augmentation procedures in localized defects in the alveolar ridge: clinical results with different bone grafts and bone-substitute materials,” Int. J. Oral Maxillofac. Implants, 2009, 24 Suppl:218-236). Nevertheless, due to the morbidity associated with them (Herford, A. S. et al., “Complications in bone grafting,” Oral Maxillofac. Surg. Clin. North Am., 2011, 23(3):433-442), bone allografts and xenografts, as well as alloplastic grafts have been used as substitutes, albeit with limited success (Jensen, S. S. et al., “Bone augmentation procedures in localized defects in the alveolar ridge: clinical results with different bone grafts and bone-substitute materials,” Int. J. Oral Maxillofac. Implants, 2009, 24 Suppl:218-236; Herford, A. S. et al., “Complications in bone grafting,” Oral Maxillofac. Surg. Clin. North Am., 2011, 23(3):433-442). For example, there are some bone graft products such as Bio-OSS®, DynaBlast®, INFUSE®, PROGENIX®, Grafton DBM and MinerOss in the market, but none of them is specifically designed for treatment of PI, nor has antimicrobial properties. Most of the trials that tested more complex and expensive therapies did not show any statistically or clinically significant advantages over deep mechanical cleaning (Inc., i.R., US Dental Bone Graft Substitutes and other Biomaterials Market. 2015: iData Research Inc.). INFUSE®, a commercially available product for bone regeneration, based on combination of human recombinant bone morphogenetic protein 2 (hrBMP2) and collagen, has also been proposed for implant re-osseointegration (Hanisch, O. et al., “Bone formation and reosseointegration in peri-implantitis defects following surgical implantation of rhBMP-2,” Int. J. Oral Maxillofac. Implants, 1997, 12(5):604-610). Yet, the uncontrolled release rate of the growth factor (Tevlin, R. et al., “Biomaterials for craniofacial bone engineering,” J. Dent. Res., 2014, 93(12):1187-1195) and the potentially harmful side effects (e.g. cancer) associated with hrBMP2 (Woo, E. J., “Adverse events reported after the use of recombinant human bone morphogenetic protein 2,” J. Oral Maxillofac. Surg., 2012, 70(4):765-767; Mesfin, A. et al., “High-dose rhBMP-2 for adults: major and minor complications: a study of 502 spine cases.,” J. Bone Joint Surg. Am., 2013, 95(17):1546-1553) severely limit its application in PI treatment. Thus, the development of approaches based on the use different growth factors or alternative osteoinductive agents to regenerate the bone around implants are highly desired. Currently, there are no commercial products that combine high adhesion to soft and hard oral tissues, and antimicrobial and osteoinductive properties; therefore, clinical management of PI remains challenging.

There are two treatment approaches:

Approach 1. Engineering visible light crosslinkable bioadhesives with osteoinductive and antimicrobial properties, through incorporation of laponite silicate nanoparticles (SN) and antimicrobial peptides within gelatin based bioadhesives (Growth factor free approach).

Approach 2. Engineering SN/VEGF-loaded bioadhesives to promote re-osseointegration. By incorporating SNs and covalently conjugating VEGF into the gel, osteoinductive and antimicrobial bioadhesives can be engineered that can induce in vitro differentiation of hMSCs to bone. By optimization of the concentration of SNs and VEGF within GelMAC-AMP hydrogel an antimicrobial and osteoinductive hydrogel adhesive for the treatment of PIs can be formed.

Recent studies have shown that vascular endothelial growth factor (VEGF) can directly induce vasculogenesis and osteoblastogenesis (Hu, K. et al., J. Clin. Invest., 2016, 126:509-526; Liu, Y. et al., J. Clin. Invest., 2012, 122:3101-3113) and that VEGF loaded hydrogels could be used to regenerate critical size bone defects (Kaigler, D. et al., J. Periodontol., 2013, 84:230-238). The osteoinductive properties of SNs also stem from their dissolution products, i.e., lithium (Li⁺), orthosilicic acid (Si(OH)₄), and magnesium (Mg²⁺) (Thompson, D. W. et al., Journal of Colloid and Interface Science, 1992, 151:236-243), which are individually shown to modulate processes related to bone tissue engineering. Therefore, the synergistic effect of SNs and VEGF is another innovative aspect of the present study to promote bone regeneration for the treatment of PI.

AMPs are small cationic and hydrophobic peptides that can inhibit or kill bacteria at very low concentrations, often by non-specific mechanisms (Ageitos, J. M. et al., Biochem. Pharmacol., 2016, 133:117-138). Thus, the appearance of resistance to AMPs is rare. In this project, the concentration of osteoinductive laponite NPs was optimized to achieve the highest bone forming capability. In addition, the concentration of AMP was optimized to achieve the highest antimicrobial activity, while maintaining the cytocompatibility of the bioadhesive hydrogels. Moreover, the inventive GelMA adhesives were able to be used as the polymeric backbone for this application.

Silicate nanoparticles (SN) were incorporated into the AMP-loaded hydrogels, as a growth factor-free strategy for osteoinductivity. Commercial products based on the use of recombinant growth factor such as rhBMP2 have been recently developed. However, the use of rhBMP2 is associated with low efficacy of peri-implant bone regeneration (30-40%), as well as serious post-operative complications, including cancer (Hanisch, O. et al., Int. J. Oral Maxillofac. Implants, 1997, 12:604-610; Woo, E. J., J. Oral Maxillofac. Surg., 2012, 70:765-767; Mesfin, A. et al., J. Bone Joint Surg. Am., 2013, 95:1546-1553). In contrast, it has been shown that SN can induce osteoblastic differentiation of human mesenchymal stem cells (hMSCs) in the absence of growth factor (Byambaa, B. et al., Adv. Healthc. Mater., 2017, 6:1700015; Cheng, H. et al., ACS Appl. Mater. Interfaces, 2017, 9:11428-11439; Wang, S. et al., Journal of Materials Chemistry, 2012, 22:23357-23367; Gaharwar, A. K. et al., Adv. Mater., 2013, 25:3329-3336; Mihaila, S. M. et al., Biomaterials, 2014, 35:9087-9099; Xavier, J. R. et al., ACS Nano, 2015, 9:3109-3118), and be degraded into nontoxic byproducts under physiological conditions. The engineered hydrogels are extremely cost effective compared to existing treatments, can be administered easily and quickly, and can be locally polymerized by FDA approved dental curing lights that are already used by dentists. The engineered hydrogels are extremely cost effective compared to existing treatments, can be administered easily and quickly, and can be locally polymerized by FDA approved dental curing lights that are already used by dentists.

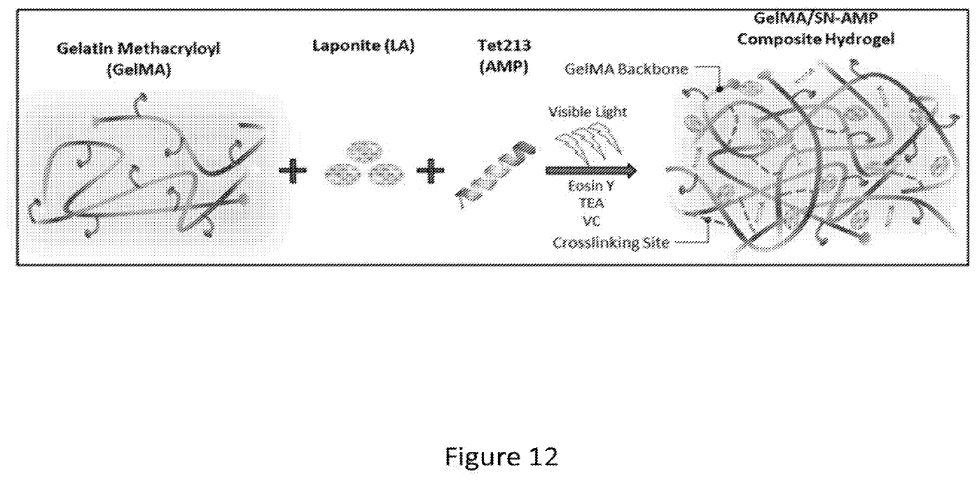

Osteoinductive silicate nanoparticles (SNs) (laponite XLG) were incorporated into GelAMP (GelMA-AMP) adhesive hydrogels (FIG. 12A) resulting in osteoinductive and antimicrobial adhesives that: 1) can be rapidly photocrosslinked in situ using dental curing lights, 2) are able to strongly adhere to soft/hard oral tissues, as well as implant surfaces in the presence of blood and saliva, 3) exhibit potent antimicrobial activity, and 4) can induce bone regeneration without the need for exogenous growth factors.

### Physical Characterization of Composite Bioadhesive Hydrogels

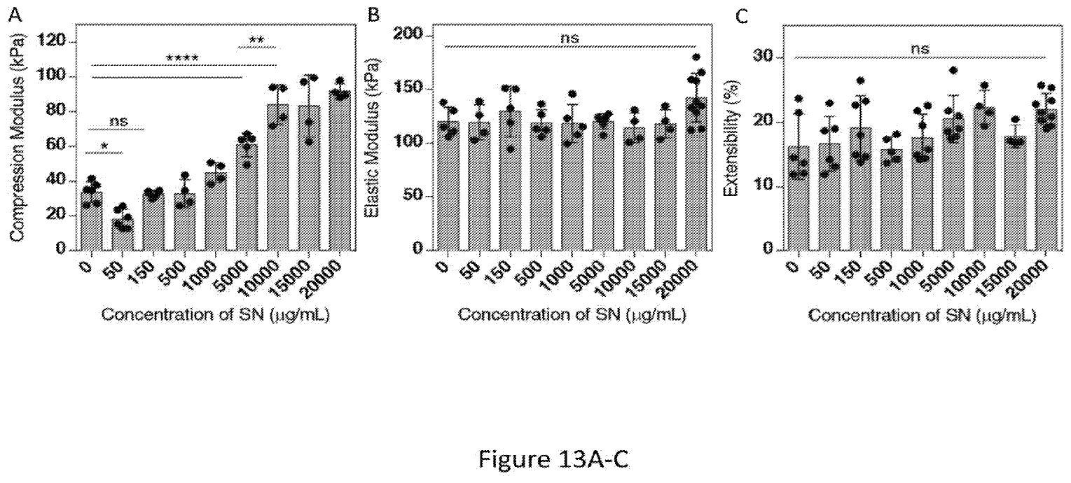

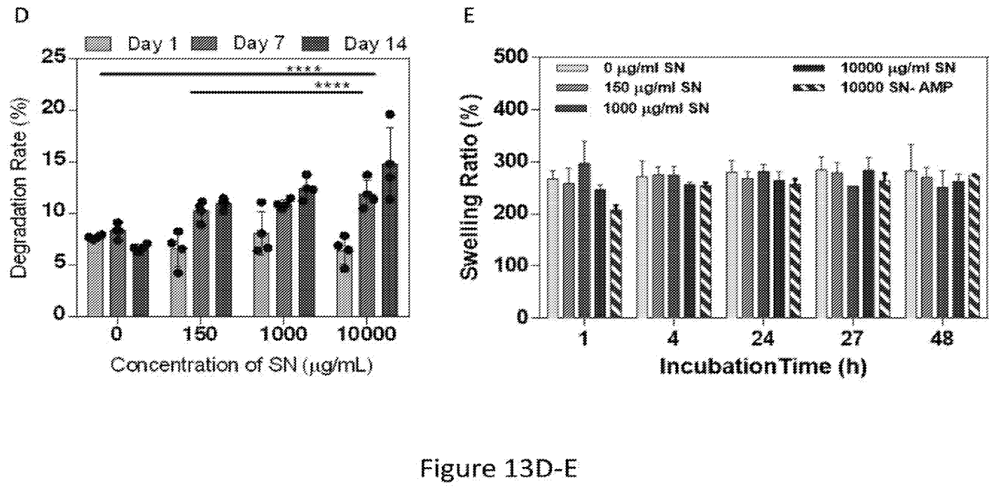

The physical properties of a library of bioadhesive hydrogels was characterized wherein the hydrogels were formed by using different concentrations of AMP and SN to optimize mechanical strength and adhesion properties of the hydrogels. The results demonstrate that the maximum compressive modulus was observed for the adhesive hydrogels containing 10000 μg/ml SN (FIG. 13A). However, the elastic modulus (FIG. 13B) and extensibility (FIG. 13C) of the hydrogels did not alter by changing SN concentration. An in vitro degradation test was performed, where the adhesive hydrogels showed containing higher SN content showed significantly higher degradation rate as compared to hydrogels without SN (FIG. 13D). Moreover, the swelling test indicated no significant difference in the water uptake capacity of the hydrogels (FIG. 13E).

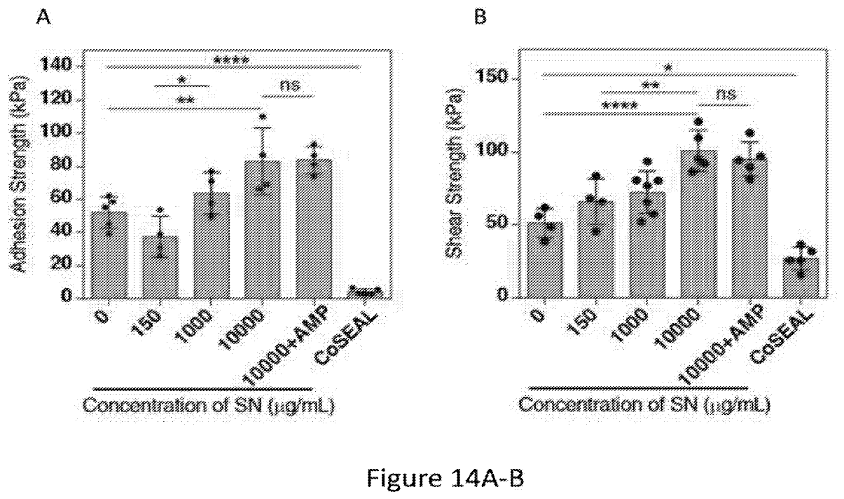

A standard Wound Closure Test (FIG. 14A) and Lap Shear Test (FIG. 14B) were performed to measure the adhesive strength of the bioadhesives. Based on the wound closure test, the bioadhesives containing 10000 μg/ml SN showed significantly higher adhesive strength to porcine gingiva, as compared to hydrogels without SN. In addition, the results of the lap shear tests showed that the adhesive strength of the bioadhesive hydrogels to titanium surfaces increased from 49.6±11.6 kPa to 99.8±18.5 kPa by increasing the SN concentration from 0 to 10000 μg/ml and both the adhesion strength and the lap shear strength of the bioadhesive were significantly higher than Coseal®) (FIG. 14A-B). The results also confirmed that the addition of AMP has no effect on the mechanical or adhesive properties of the hydrogels.

### In Vitro Antimicrobial Properties of Composite Bioadhesive Hydrogels

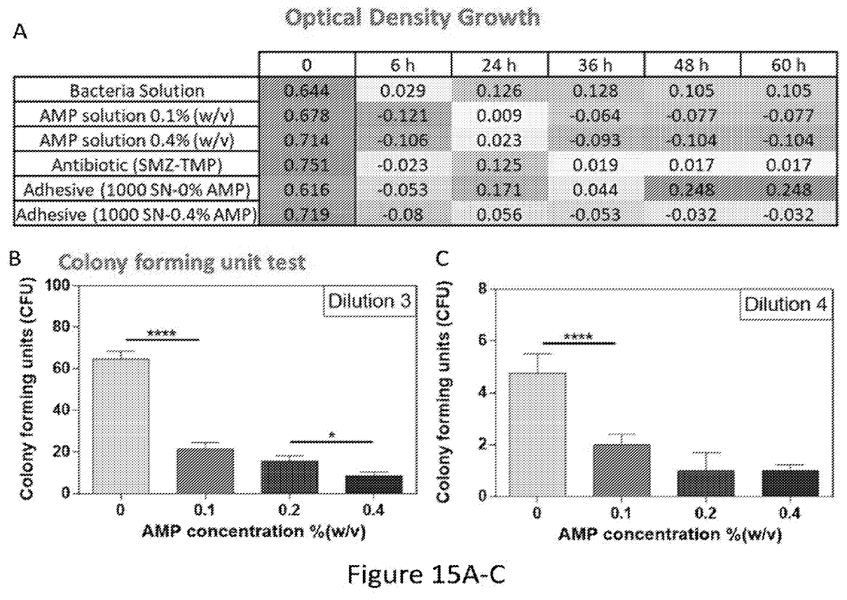

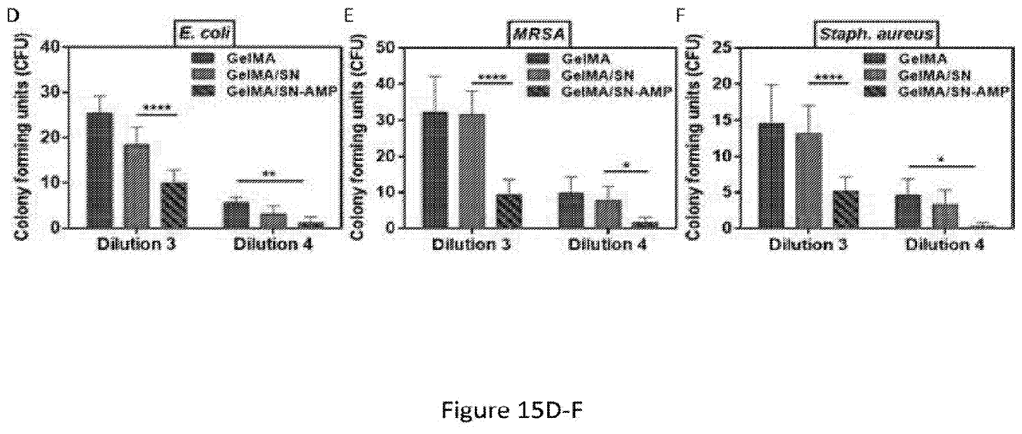

In addition, the antimicrobial activity of AMP was evaluated using different anaerobic and aerobic bacteria (G+/−). First, the antimicrobial activity of the resulting bioadhesive against P. gingivalis was evaluated using standard optical density (OD) growth test, and colony forming units (CFU) assay (FIG. 15A-F). The OD test revealed that the optical density of the samples containing 0.4% (w/v) AMP, as well as AMP solution (same concentration) decreased significantly overtime as compared to the samples without or with lower concentration of AMP, as well as sulfamethoxazole/trimethoprim (SMZ-TMP) antibiotic as control (FIG. 15A). The CFU assay showed that the number of P. gingivalis colonies in the 3-logarithmic dilution decreased significantly at 0.4% (w/v) AMP, as compared to 0, 0.1, and 0.2% (w/v) AMP (FIG. 15B). A similar response was also observed for the 4-logarithmic dilution (FIG. 15C). In addition, the antimicrobial activity of the hydrogels was examined against three different aerobic bacteria (G+/−), including multidrug resistant (MDR) E. coli, MRSA, and Staphylococcus aureus (FIG. 15D-F). The results demonstrated that the samples containing 0.4% AMP showed the highest antimicrobial activity against all three bacteria, as compared to GelMA/SN and pristine GelMA hydrogels (FIG. 15D-F).

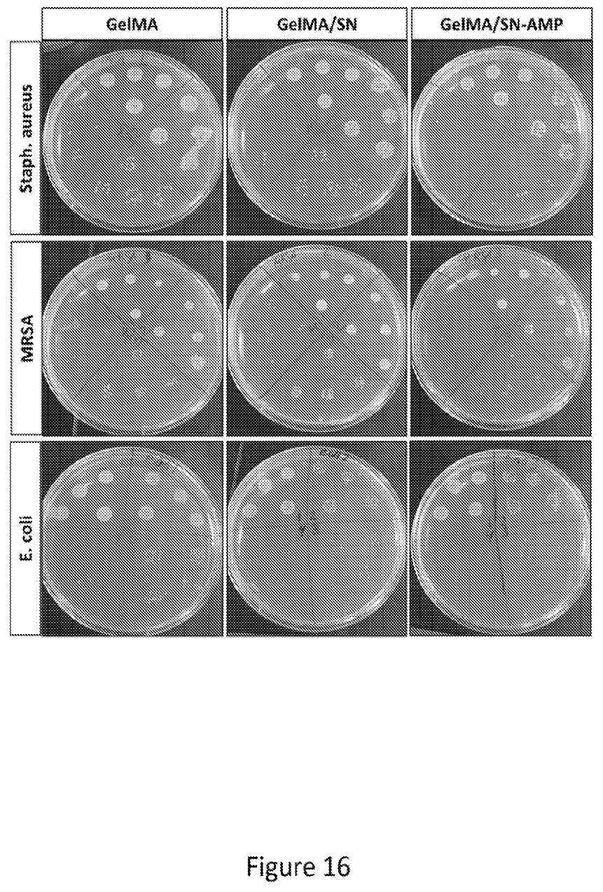

FIG. 16 depicts the in vitro antimicrobial properties of the bioadhesive hydrogels against three different G+/− aerobic bacteria. Specifically, FIG. 16 depicts representative images of bacterial colonies grown on agar plates for bioadhesives with and without AMP and SN (Dilution 1, 2, 3 and 4 represent 1-, 3- and 4-logarithmic dilutions respectively).

### In Vitro Cytocompatibility and Differentiation Studies

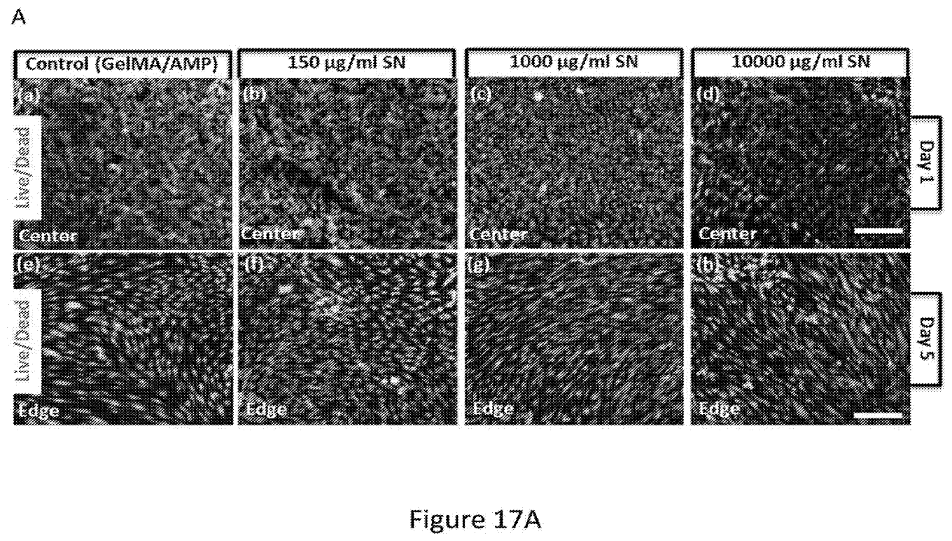

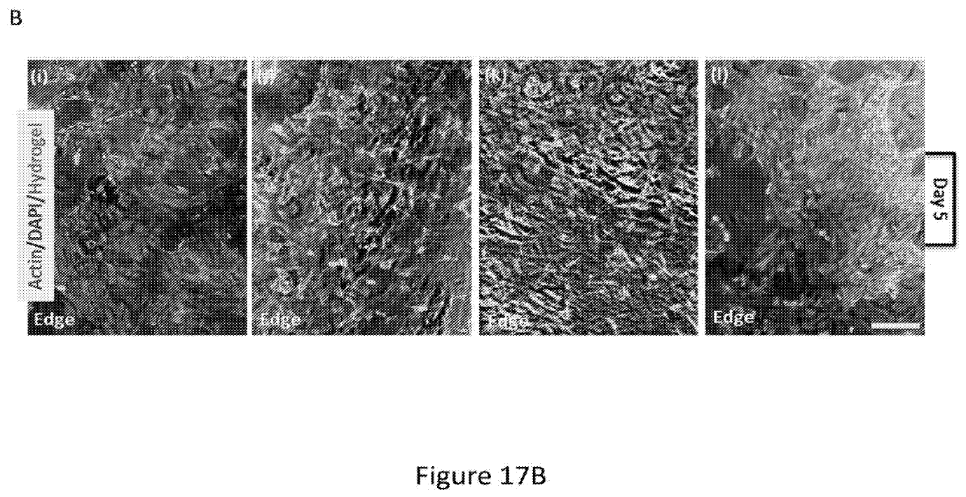

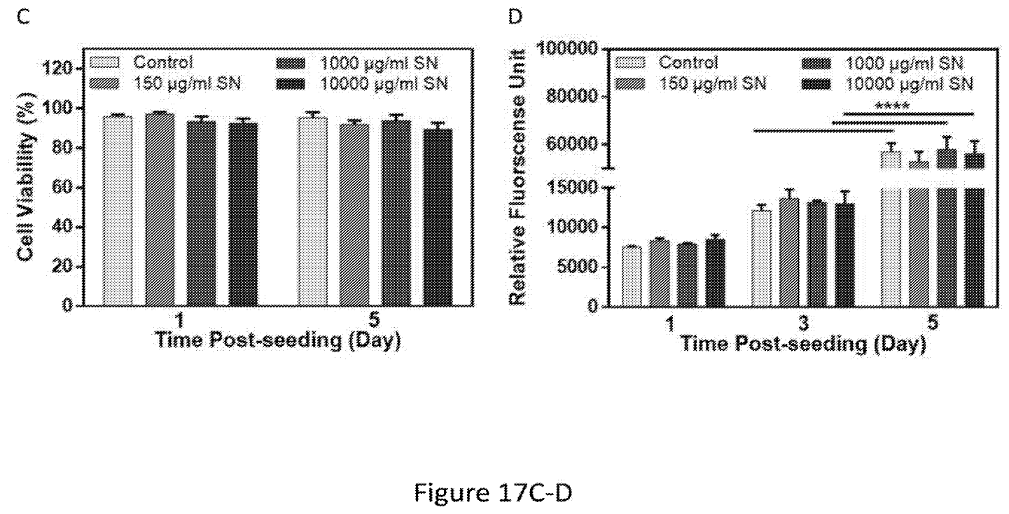

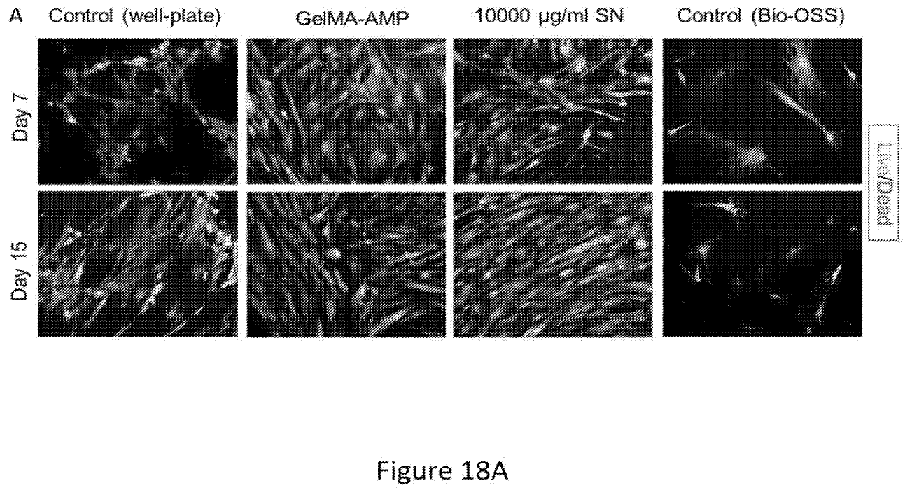

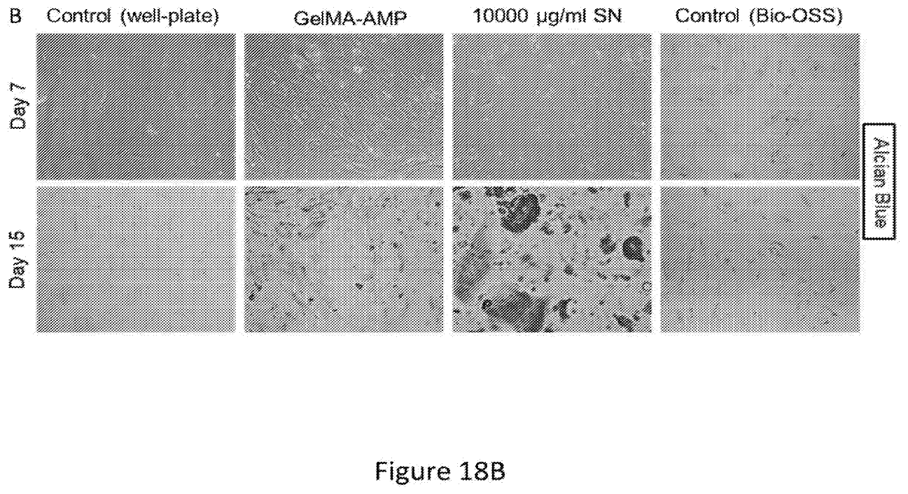

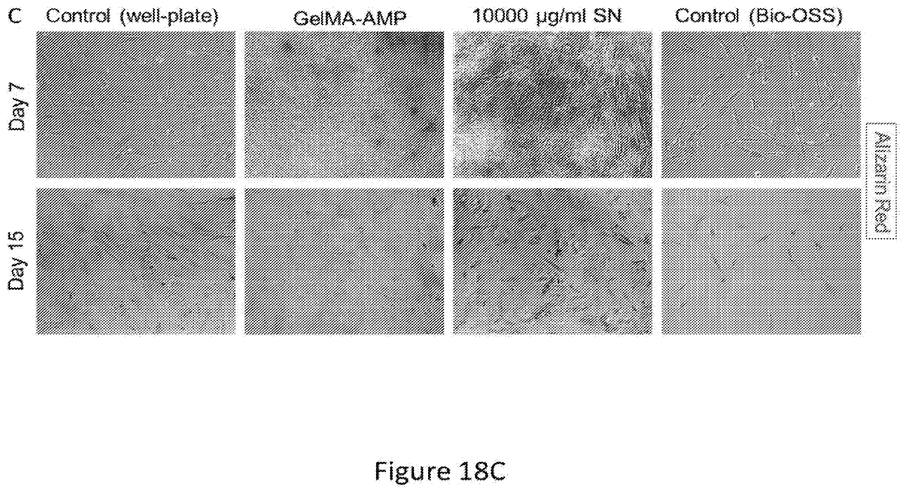

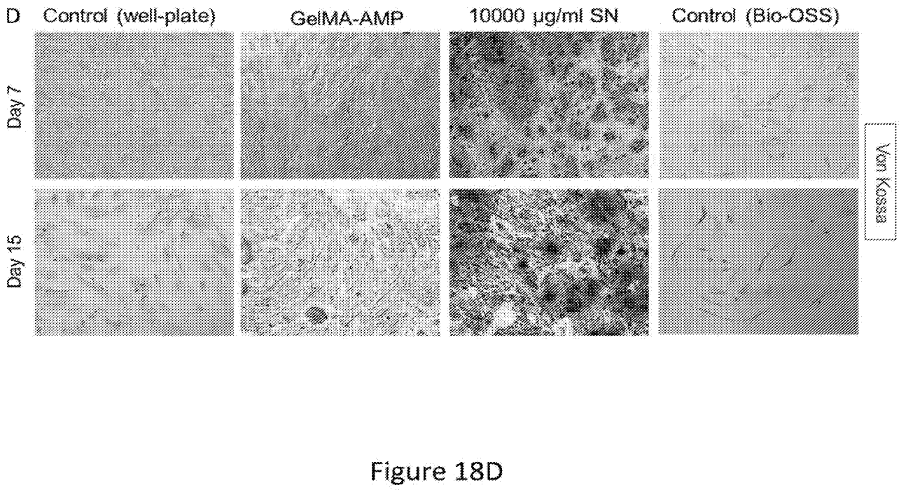

In this section, different in vitro assays were used to evaluate hMSCs viability, proliferation, and differentiation. According to live/dead assay, cell viability was >90% for all the samples for up to 5 days post seeding (FIG. 17A and FIG. 17C). In addition, hMSCs were able to spread on the surface of the hydrogels based on Actin/DAPI staining (FIG. 17B). Moreover, the cell metabolic activity, measured by PrestoBlue assay, increased consistently for all the samples over time (FIG. 17D). Next, the osteogenic differentiation of hMSCs seeded on the surface of the hydrogels with and without SNs was evaluated. Two control groups were used: well-plate with and without Bio-OSS bone graft. The results indicated that the hMSCs seeded on bioadhesive hydrogels showed the highest viability, adhesion and spreading after 15 days as compared to control groups (FIG. 18A). In addition, the results showed higher chondrogenic differentiation of hMSCs seeded on bioadhesives containing SNs after 15 days of culture (FIG. 18B). The Alizarin staining also showed higher calcium deposition, indication higher osteogenic differentiation of hMSCs seeded on bioadhesives containing SNs after 7 and 15 days of culture as compared to other groups (FIG. 18C). Representative images of Von Kossa staining for hMSCs seeded on the surface of bioadhesive hydrogels containing AMP, with SNs showed higher phosphate deposition, indication higher osteogenic differentiation of hMSCs seeded on bioadhesives containing SNs after 7 and 15 days of culture as compared to other groups (FIG. 18D).

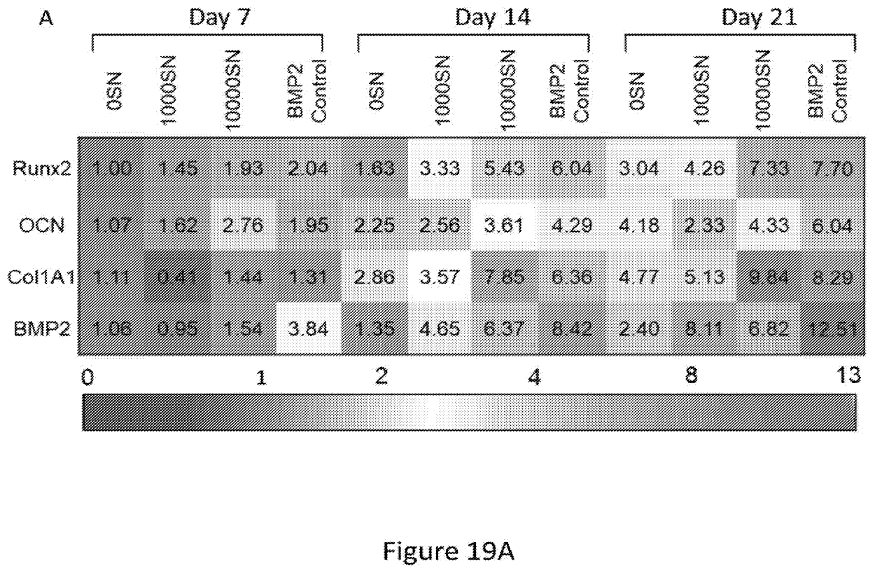

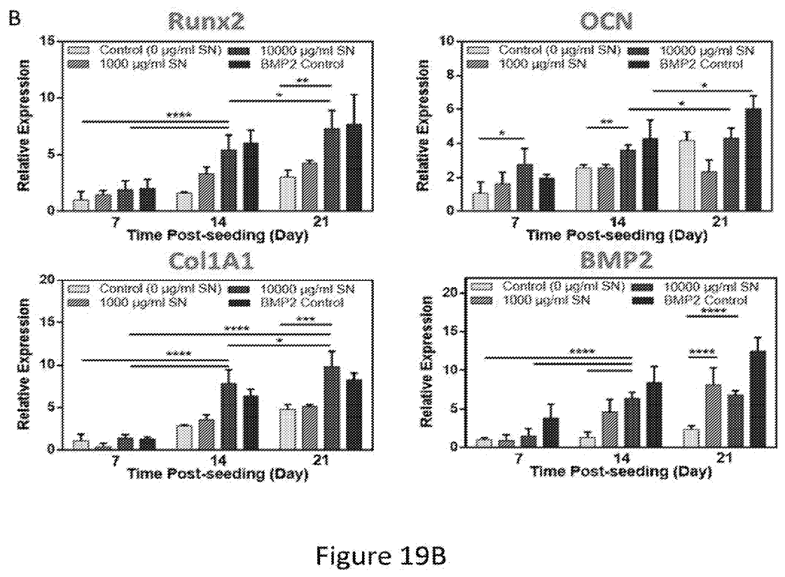

FIGS. 19A-B depict RT-PCR analysis of in vitro differentiation of hMSCs seeded on bioadhesive hydrogels. FIG. 19A depicts a chart showing the quantification of gene expression for hMSCs seeded on bioadhesive hydrogels formed with different concentrations of SN and compared to BMP2 treated cells as control. FIG. 19B depicts the data showing the quantification of gene expression for hMSCs seeded on bioadhesive hydrogels formed with different concentrations of SN and compared to BMP2 treated cells as control.

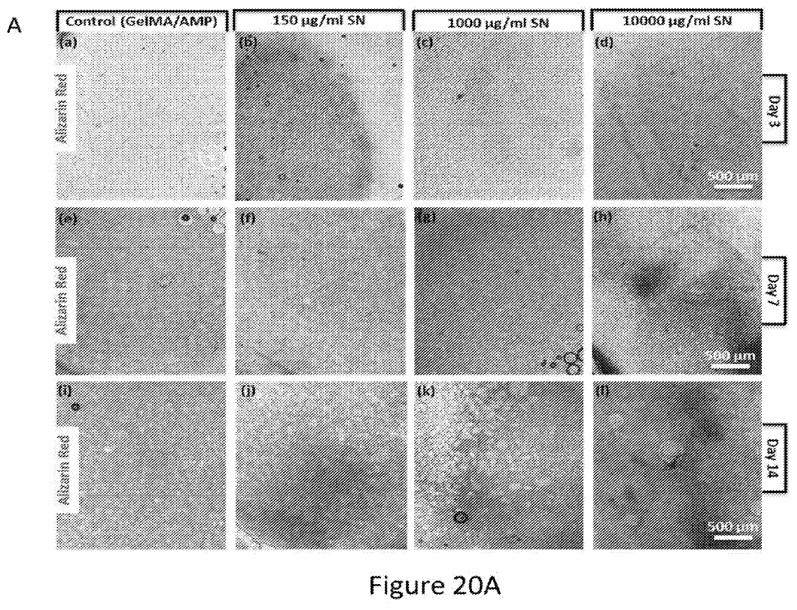

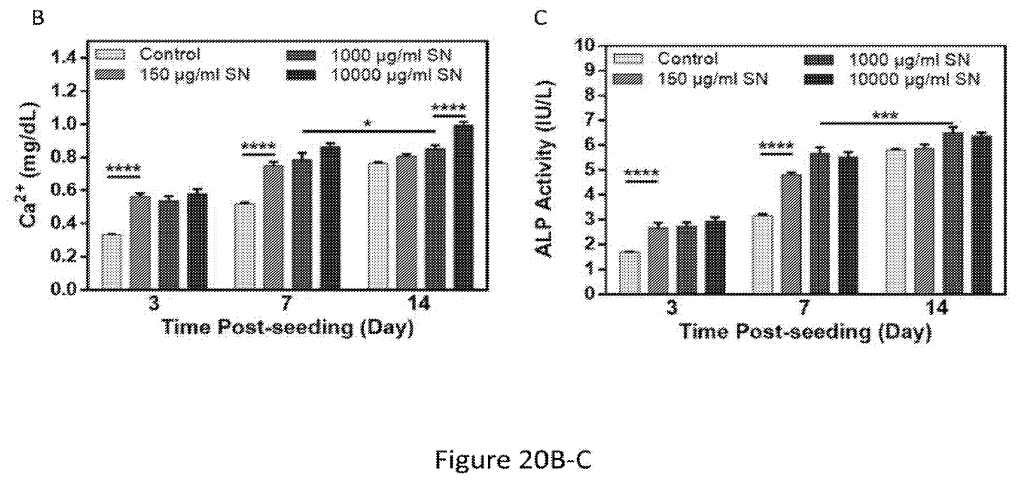

FIG. 20 depicts the in vitro differentiation of w-20-17 cells seeded on bioadhesive hydrogels. FIG. 20A depicts representative images of Alizarin red staining for w-20-17 cells seeded on the bioadhesive hydrogels containing different concentrations of SN. FIG. 20B depicts the quantification of Ca′ deposition for w-20-17 cells seeded on the bioadhesive hydrogels containing different concentrations of SN. FIG. 20C depicts the quantification of alkaline phosphatase assays for w-20-17 cells seeded on the bioadhesive hydrogels containing different concentrations of SN.

### In Vivo Biocompatibility and Biodegradation of Composite Bioadhesives

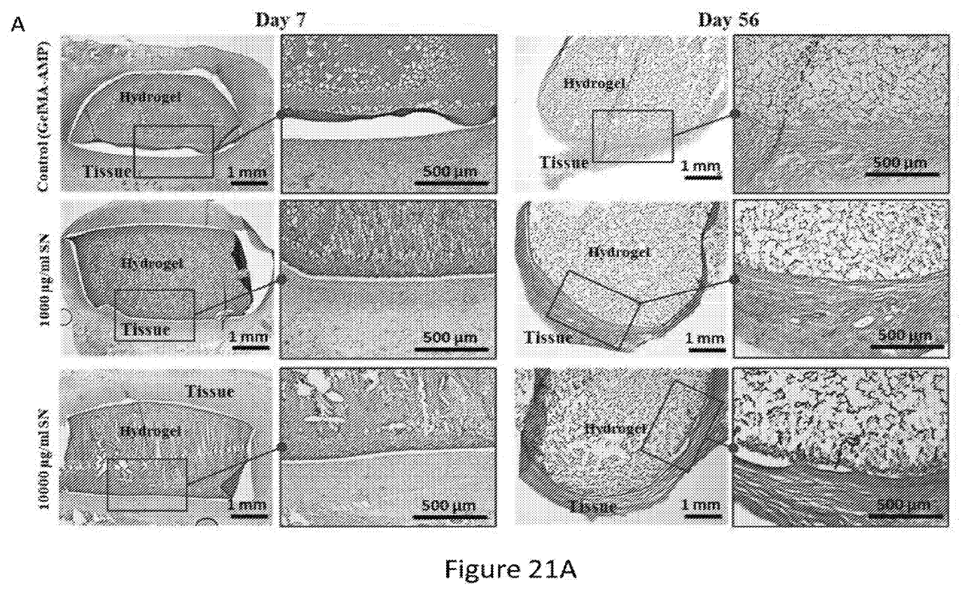

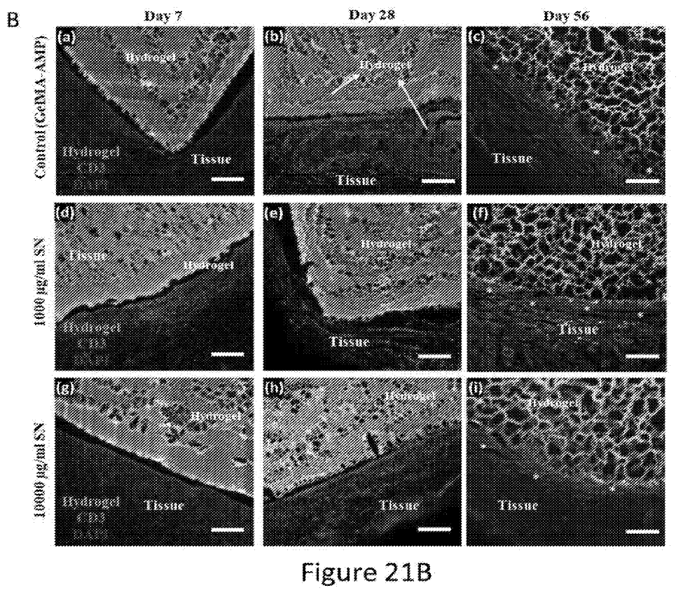

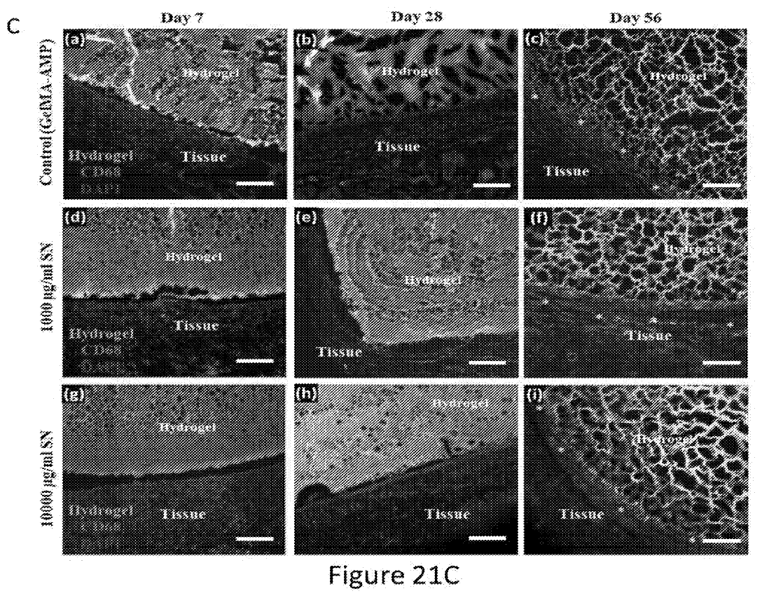

In order to assess biocompatibility and biodegradation of bioadhesive hydrogels, a rat subcutaneous implantation model was used (as described before). The results of subcutaneous implantation showed that the implanted bioadhesives (both formulations with and without SN) could be efficiently biodegraded in a period of time that allowed the growth of new autologous tissue after 56 days post implantation (FIG. 21A). Immunofluorescent analysis of subcutaneously implanted bioadhesive hydrogels with different concentrations of SN, explanted at day 7, and day 56 showed minor local lymphocyte infiltration (CD3) at 7 and 28 days post implantation (FIG. 21B). At 56 days post implantation, the lymphocytes had cleared from the region of implantation for all the hydrogels. Immunofluorescent analysis of subcutaneously implanted bioadhesive hydrogels with different concentrations of SN, explanted at day 7, and day 56 showed minor macrophage infiltration (CD68) at 7 days post implantation (FIG. 21C). At 56 days post implantation, the macrophage had cleared from the region of implantation for all the hydrogels.

### In Vivo Bone Forming Capacity of Composite Bioadhesives

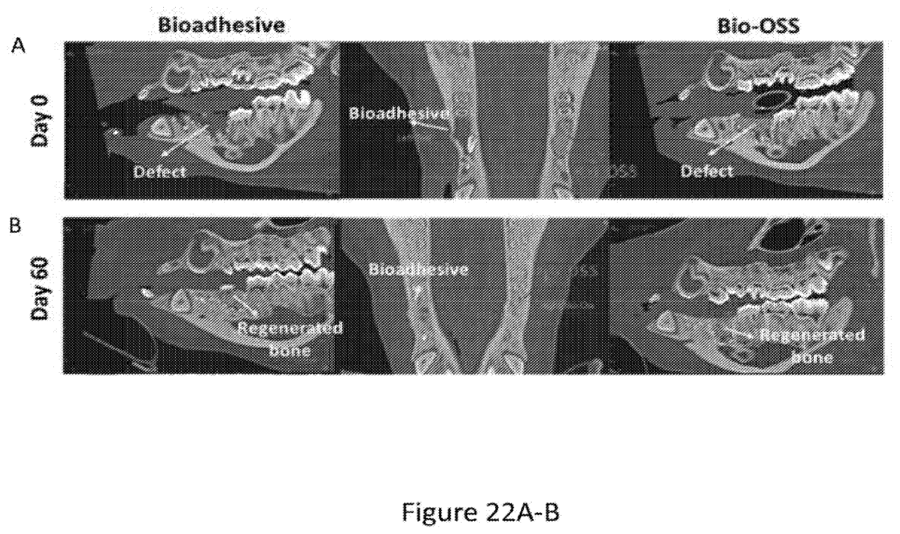

Next, the bone forming capability of the adhesive hydrogel containing AMP and SN was tested in a large mandibular bone defect model in miniature pigs (FIG. 22A-B). Commercial bone graft Bio-OSS was used as a control. The defect treated with Bio-OSS bone graft showed high bone density at day 0 (FIG. 22A). This is mainly due to radiopaque nature of Bio-OSS granules. After 60 days, the new bone formed on the surface of Bio-Oss and bridged the particles, (FIG. 22B), which is similar to previous reports (Pietruska, M. D., “A comparative study on the use of Bio-Oss® and enamel matrix derivative (Emdogain®) in the treatment of periodontal bone defects,” Euro. J. Oral Sci., 2001, 109(3):178-181; Wong, R. et al., “Effect of Bio-Oss® Collagen and Collagen matrix on bone formation,” Open Biomed. Eng. J., 2010. 4:71-76). In contrast, the adhesive hydrogels were radiolucent at day 0, while appeared to show extensive new bone formation and complete bone filling the defect after 60 days (FIG. 22A-B). In addition, unlike Bio-OSS granules, bioadhesive precursor could readily fill the holes in the defect site due to the injectable nature of the prepolymer solution. There are also some reports on limited bioresorption of Bio-OSS in vivo, where the Bio-OSS remnants could be detected in the bone defect after long term implantation (>44 months) (Duda, M. et al., “The issue of bioresorption of the Bio-Oss xenogeneic bone substitute in bone defects,” Annales Universitatis Mariae Curie-Sklodowska. Sectio D: Medicina, 2004).

### In Vivo of Composite Bioadhesives in a Ligature Induced Peri-Implantitis Model in Miniature Pigs

### Experiment 1

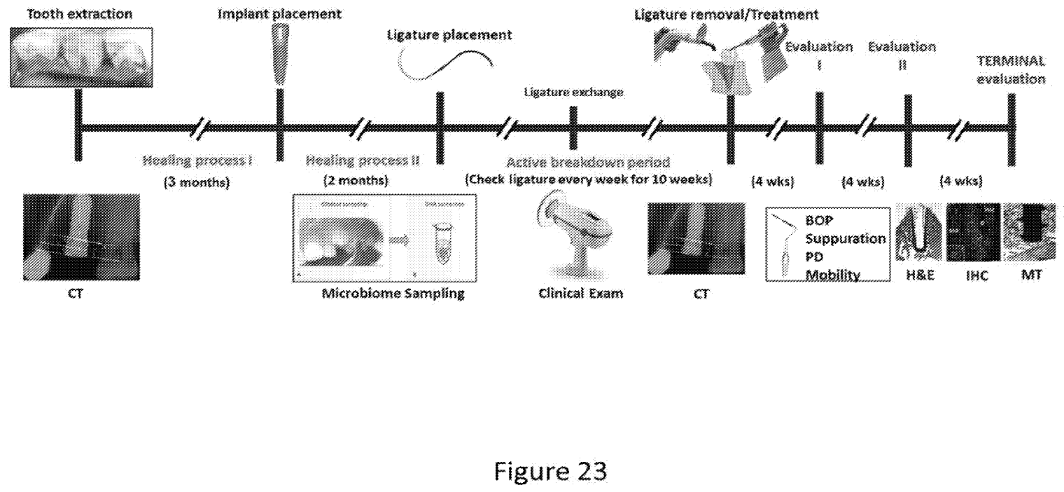

3 miniature pigs 12 months of age (average body weight=55 kg) were utilized. Two months after the bilateral removal of all premolars, three titanium implants per side were inserted into each animal (4 implants/animal). Two months after the insertion of the implants, peri-implantitis (PI) was induced by placing a ligature around the neck of the implants, allowing for the undisturbed accumulation of plaque and calculus around the implants. Ligatures were replaced after 2 months and then in a timely manner to develop PI as bone loss. Then treatments were initiated, and evaluations were performed every 4 weeks (FIG. 23). The following treatments were tested: 1) no treatment/with ligature, 2) PI treated with mechanical debridement+chemical decontamination+commercial bone graft (Bio-Oss®, Geistlich Pharma AG), 3) PI treated with mechanical debridement+chemical decontamination+GelMA-AMP, 4) PI treated with mechanical debridement+chemical decontamination+GelMA-AMP-Silicate NPs. After surgical exposure of the PI defects and mechanical debridement, treatments (100-200 μl) were delivered by means of a sterile syringe with a 20-gauge blunt tip needle to fill the peri-implant defects. The GelMA-based precursors were then polymerized in situ with a VALO® LED curing light using the exposure time optimized before. Bio-Oss® has been chosen as control, since it is a current gold standard for treatment of periodontal bone loss, leading bone substitute for regenerative dentistry worldwide, and has been widely used for PI treatment (British Dental Association, Peri-implant diseases, BDA evidence study, 2015).

### Study Assessments

Clinical, radiological, microbiological, and histological analysis: For all groups parameters that quantify bone regeneration were evaluated: 1) longitudinally, by means of radiological evaluations; and 2) cross-sectionally, by means of histometric evaluations (terminal evaluation of implants with treated PI). For radiological analysis, standardized X-rays and high resolution computed tomography (CT) images were obtained and defect depth (DD, as the distance from the implant shoulder to the most apical aspect of the peri-implantitis defect), bone reconstruction height (BRH, as the distance from the most apical aspect of the peri-implantitis defect to the most coronal new bone implant contact), bone reconstruction area (BRA, as the area of the newly formed bone within the peri-implantitis defect) was measured. For histometric evaluations, the lengths of the junctional epithelium (JE) and sulcular epithelium (SE), and Total Osseointegration (TO, as the fraction of the bone—implant contact (BIC) from the most apical aspect of the implant to the implant shoulder) were measured. Measurements were performed at the mesial and distal implant aspects of each X-Ray, CT scan, and histological slice and the mean of each measurement was calculated for each time point/group. To assess the quality of newly formed bone, back-scattered electron microscopy analysis was performed (Slater, N. et al., Clinical Oral Implants Research, 2008, 19:814-822; Lindgren, C. et al., Clinical Oral Implants Research, 2010, 21:924-93). For Microbiological evaluations, samples of sub-gingival plaque were collected, at the same points used for clinical evaluations. Then, the microbial composition of plaque samples was characterized by sequencing the hyper-variable V3 and V4 regions of the 16S rDNA gene using the Illumina MiSeq® platform (Lee, C. T. et al., J. Immunol., 2016, 197:2796-2806). For Histological evaluations, after euthanasia, mandibles were collected, and fixed and histological sagittal sections were prepared for histometric analysis as described in a previous work (Hasturk, H. et al., J. Periodontol., 2014, 85:1230-1239). Lengths of the junctional epithelium (JE) and sulcular epithelium (SE) were measured at the mesial and distal points of each histological sagittal section and the mean of each measurement was calculated for each time point/group.

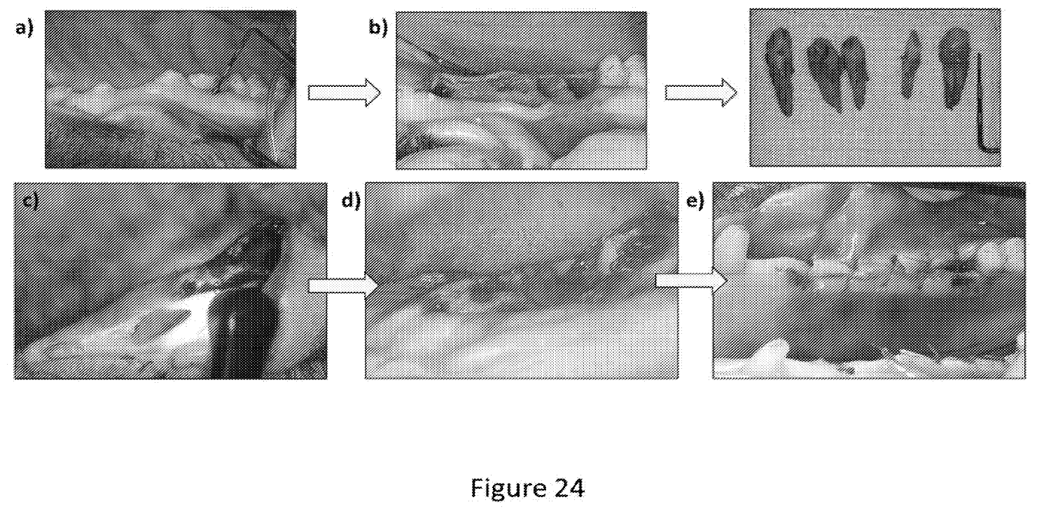

FIG. 24 depicts the in vivo application of the adhesives for treatment of large mandibular bone defects in minipigs.

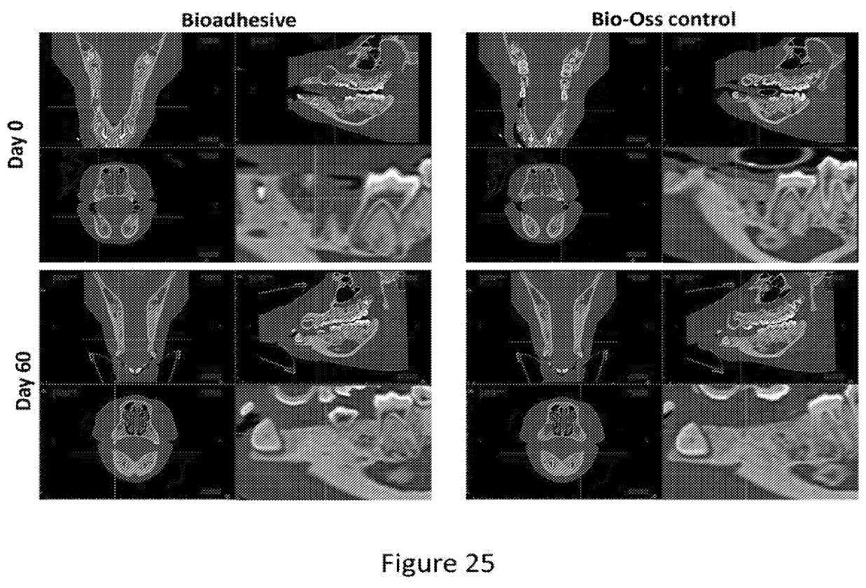

FIG. 25 depicts the in vivo application of bioadhesive hydrogels and Bio-Oss commercial bone graft in a critical sized bone defect model in miniature pigs.

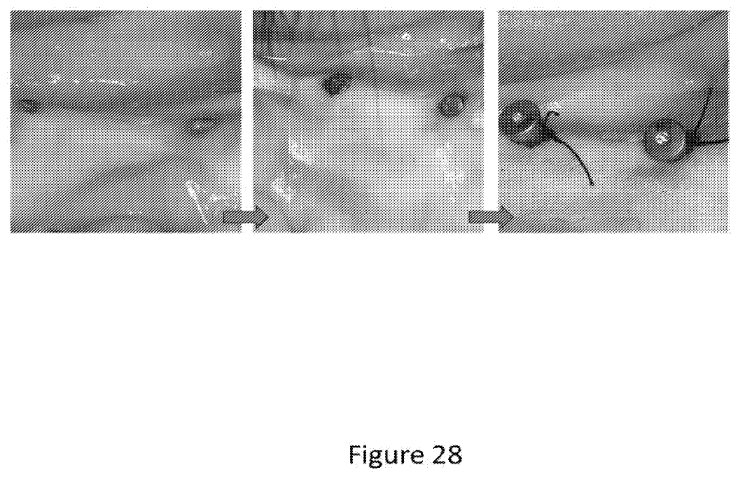

The in vivo comparison of the inventive bioadhesive hydrogels with Bio-Oss was obtained by extracting three teeth and creating a large defect in the mandible. Next, the hydrogel precursor was applied and photopolymerized with a commercial dental light curing system for 2 min. The results show full regeneration of the defect for both bioadhesive hydrogels and Bio-Oss after 60 days (FIG. 28). Bioadhesive hydrogels were formed with 0.4% (w/v) AMP and 1% (w/v) (10000 μg/ml) SN.

### Experiment 2

In order to evaluate the efficacy of bioadhesive hydrogels, a ligature induced peri-implantitis model was developed in miniature pigs and the resulting PI associated bone defects were then treated using the inventive composite bioadhesives (FIG. 23).

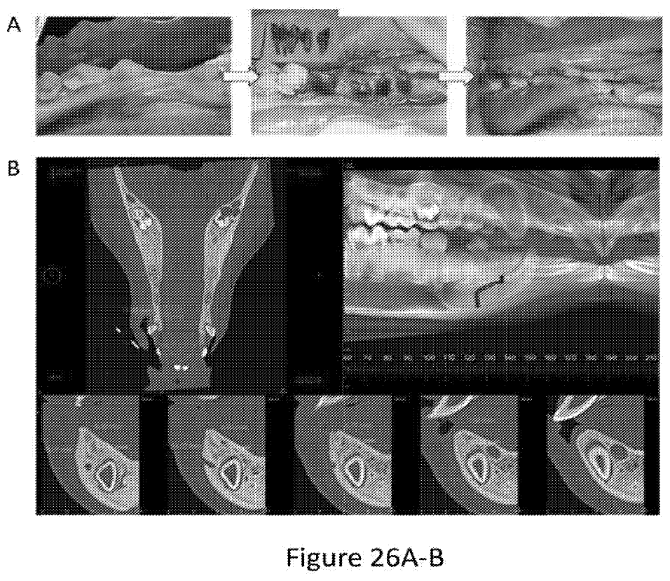

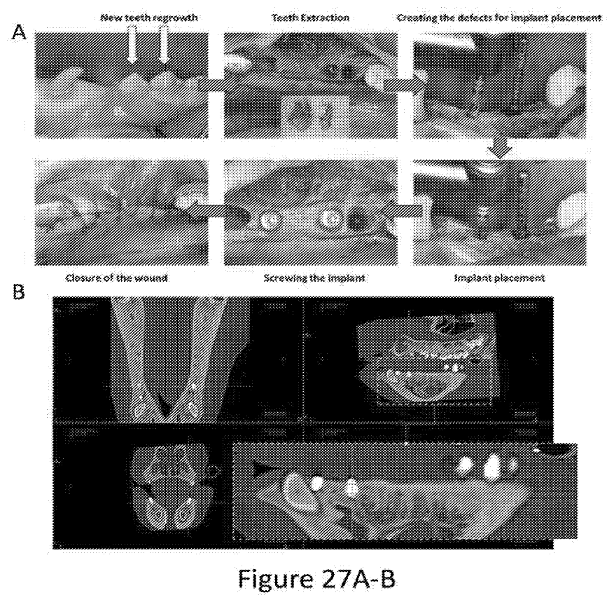

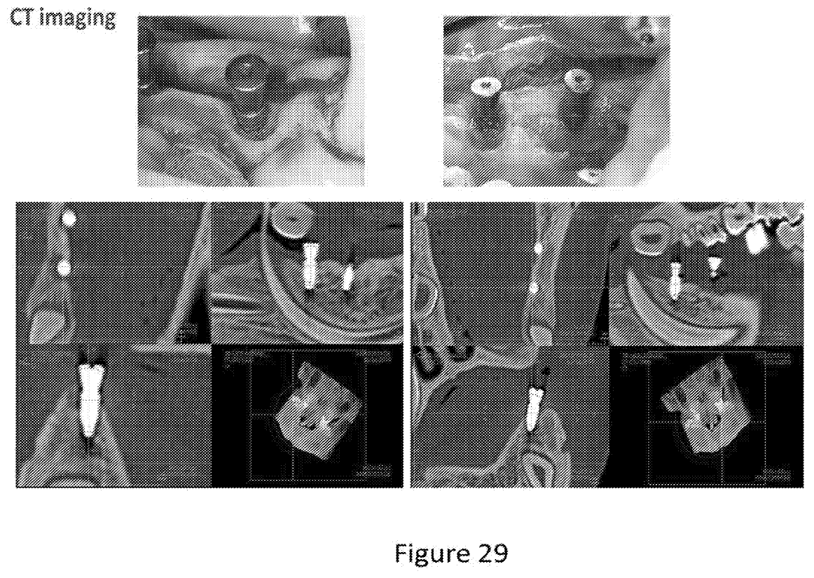

To develop the PI model, three miniature pigs (Yucatan breed, males), 12 months of age with an average body weight of approximately 65 kg were utilized. In the first step, three premolar teeth from each side of the jaw was removed (FIG. 26A). Two months after teeth extraction, new tooth growth was observed in the defect sites (FIG. 26B). Three months after the bilateral removal of the premolars, two titanium implants per side was inserted into each animal (4 implants/animal) (FIG. 27A-B). Three months after the insertion of the implants, PI was induced by placing a ligature around the neck of the implants, allowing for the undisturbed accumulation of plaque and calculus around the implants (FIG. 28). Ligatures were checked weekly and were replaced every 4 week and then left in place for a total of 3 months. During a breakdown period of 3 months, PI was developed as a significant bone loss was occurred around the dental implants (FIG. 29).

Then treatments were initiated, and evaluations were performed after 4 and 12 weeks. The following treatments were tested:

1) mechanical debridement without treatment;

2) PI treated with mechanical debridement+GelMA-AMP-SNs;

3) PI treated with mechanical debridement+Dynablast (commercial bone graft).

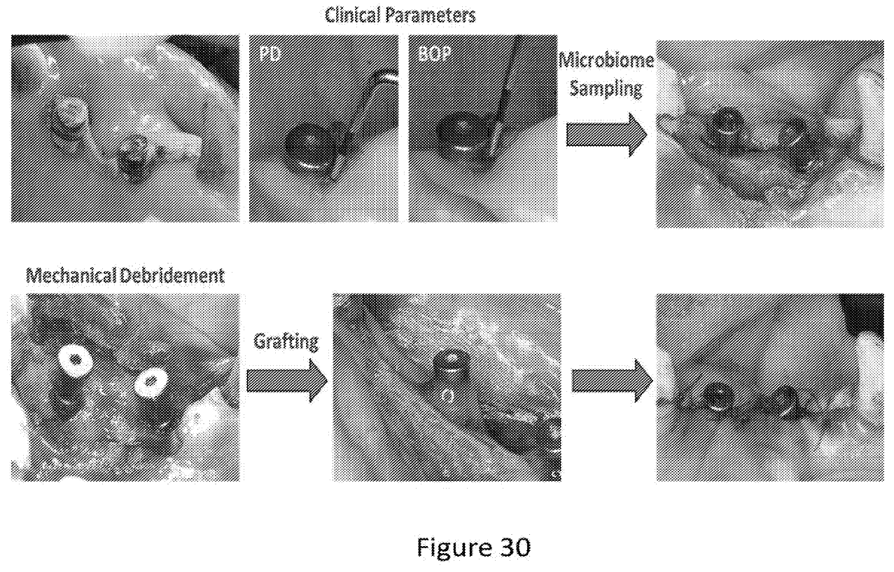

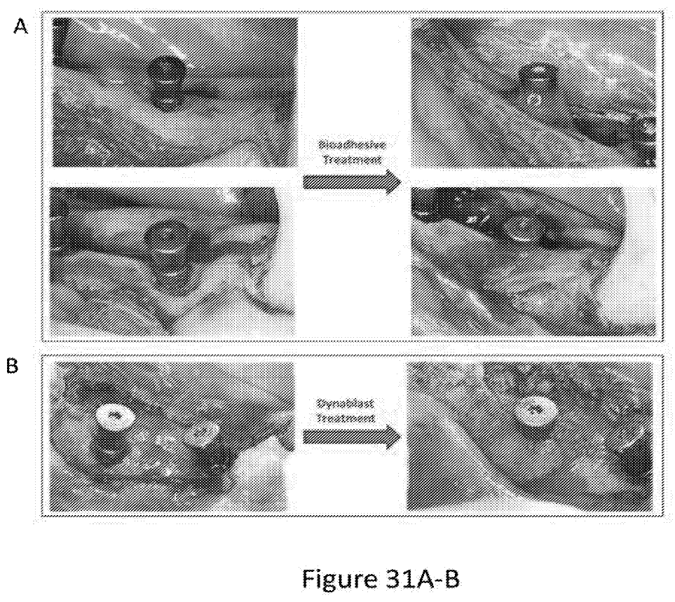

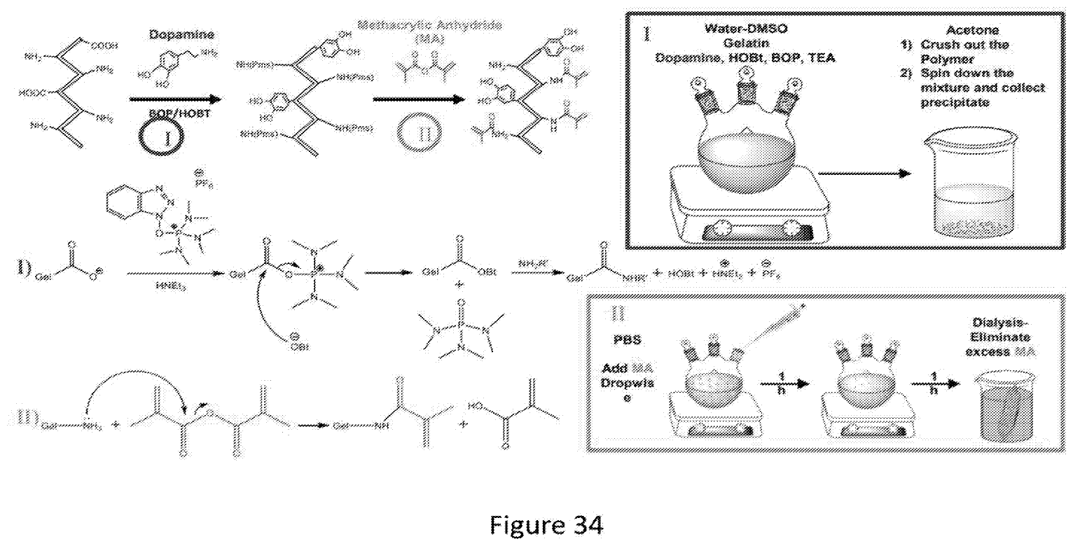

After surgical exposure of the PI defects and mechanical debridement, hydrogel treatment (˜500 μl) was pipetted to fill the peri-implant defects. Then the precursor was polymerized in situ with a VALO® LED curing light for 2 min (FIG. 30 and FIG. 31A-B). Similarly, for Dynablast bone graft, the material was transferred to the defect site by using a sterile spatula (FIG. 34A-B).

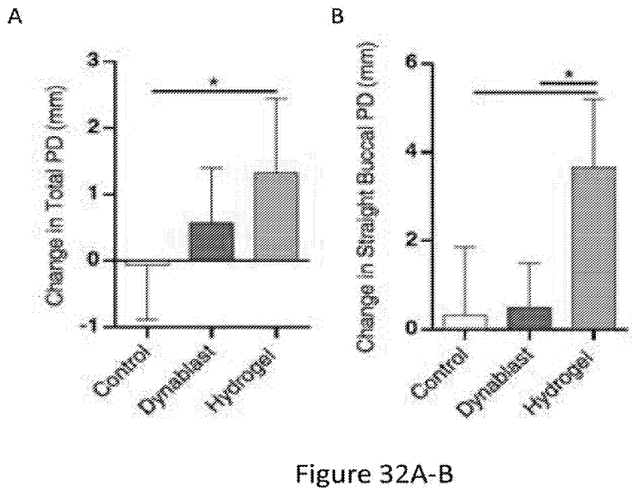

A significant bone loss was observed around the implants after one week and also one month (no significant bone regeneration) for the control group. The prosthetic parameters were measured before and after treatment. The data showed a significant decrease in total and straight buccal probing pocket depth (PD) for the implants treated with bioadhesive hydrogels as compared to untreated group and the defects treated with Dynablast (FIG. 32A-B).

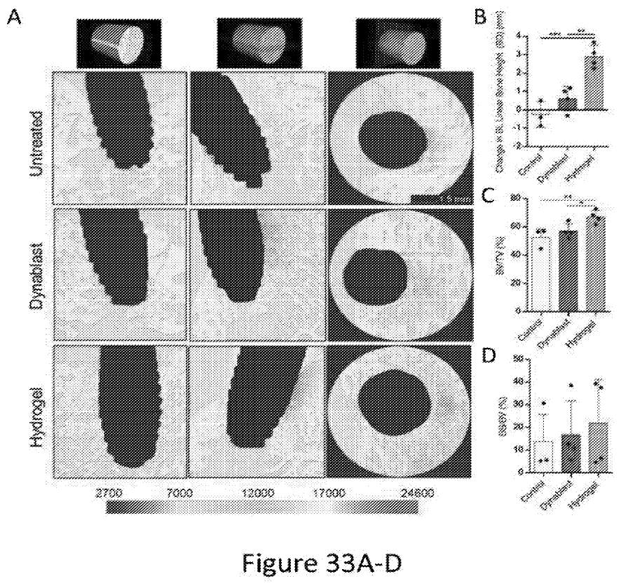

In addition, a significant bone regeneration around the implant was observed after 3 months in the distal side of the implant (height decreased from 2.48 to 1.53 mm) (FIG. 33). The height of lingual bone slightly decreased (3.45 to 3.34 mm) (FIG. 33B). FIG. 33A shows the micro-CT images for the bone defects treated with bioadhesive hydrogels, and Dynablast and untreated controls. The quantification of bone morphological parameters and trabecular analysis were performed for all the samples. Based on the results, the bone volume fraction (BV/TV) in the region of interest for the samples treated with bioadhesive hydrogels were significantly higher than those treated with Dynablast and untreated control (FIG. 33C). This clearly shows the higher bone regenerative capability of bioadhesive hydrogels even compared to a commercial bone graft (Dynablast). Bone surface density (BS/BV) was also calculated for all the samples. However, there was no statistical difference between bone surface density in all the samples (FIG. 33D).

### Conclusion

While traditional surgical closure and treatment of tissue defects is achieved by sutures, staples, or wires, the application of adhesives for different types of lesions is essential. The repair of parenchymatous defects, such as in the lungs, liver, or kidney, as well as hard tissues such as cartilage, bone and tooth is particularly challenging since the consistency of these tissues does not facilitate strong fastening of sutures or staples or due to non-healing nature of the tissue. An ideal tissue adhesive for wound closure and treatment of soft and hard tissues should be (i) biocompatible and biodegradable, (ii) rapidly crosslinked and easily applicable, (iii) antimicrobial and impervious to antibiotic resistance and to prevent biofilm formation, (iv) strongly adhesive, (v) tunable and long lasting, and (vi) possessing optimal mechanical properties and degradation rate to allow new tissue ingrowth. Therefore, new biomaterial-based approaches are needed to address the limitations of currently available alternatives. Here, a new class of photocrosslinkable biomaterials was introduced that can be easily applied to the defect sites and enhance the healing process. The photocrosslinkable adhesive biomaterials developed are antimicrobial (by incorporation of metal oxide NPs or AMPs) and can be used for both hard and soft tissue regeneration. In addition, it was shown that SNs can be incorporated into the adhesive hydrogels to induce osteoinductive functionality of the engineered hydrogels for treatment of peri-implantitis or periodontal bone defects. The engineered hydrogel adhesives could be readily delivered to the affected area and be photocrosslinked in situ using commercially available dental curing light units. In addition, studies confirmed that the incorporation of AMPS to adhesive hydrogels could effectively prevent bacterial colonization using Porphyromonas gingivalis, which is the main etiologic agent associated with chronic periodontitis. Moreover, the incorporation of SNs has been shown to promote the osteogenic differentiation of human mesenchymal stem cells (hMSCs) in vitro. Extensive new bone formation and complete healing of critical sized mandibular bone defects in miniature pigs after 8 weeks was demonstrated. Lastly, the engineered SN-loaded GelMA-AMP adhesives were used to successfully treat PI and promote re-osseointegration in vivo, using large animal (miniature pigs) model of ligature induced PI. This can yield to new and promising therapeutic approaches for treatment of PI. In summary, the multifunctional adhesive hydrogels can be used for different tissue engineering applications.

### Example 3. Synthesis and Characterization of Wet Tissue Bioadhesive for Dental Applications

Various types of hydrogel adhesives have been used for sealing, reconnecting tissues, or implant coating. However, their poor mechanical properties and adhesion to wet tissues have limited their successful implementation in the clinic. Inspired by the superior ability of mussels to adhere to wet surfaces, the critical role of the modified amino acid dihydroxyphenylalanine (DOPA) in this mechanism has been identified (Yu, J. et al., Nat. Chem. Biol., 2011, 7:588-590; Waite, J. H. et al., The Journal of Adhesion, 2005, 81(3-4):297-317). Thus, the engineering of DOPA-modified hydrogels with high adhesion to titanium implant surfaces and soft tissues, could promote compartmentalized tissue healing and would constitute a paradigm shift in the treatment of peri-implant diseases (PIDs). Therefore, photocrosslinkable polymers such as methacrylated gelatin (GelMA) hydrogels can be functionalized with DOPA to form GelMAC hydrogels with strong adhesive properties, even in the presence of blood and saliva. The engineered hydrogels are injected around the implant and crosslinked in situ using commercially available dental curing lights, making engineered system highly versatile. The hydrogel adhesive can readily take the shape of the defect site, providing a proper fit and interface between the implant and the tissues. Although a DOPA modified gelatin based coating for orthopedic implants has been studied (Cheng, H. et al., ACS Appl. Mater. Interfaces, 2017, 9:11428-11439), there are currently no commercially available products that possess strong adhesion to soft/hard oral tissues, as well as high antimicrobial and osteoinductive properties for the treatment of PIDs.

The new chemistry developed herein is based on a double crosslinking and highly stiff three-dimensional (3D) network formation. Accordingly, the adhesive hydrogels undergo two different crosslinking steps; (i) crosslinking with metal^{2+/3+} ions (i.e. Fe³⁺, Fe²⁺, Ni²⁺, Zn²⁺. . . ) through formation of metal-ligand coordination bonds, and (ii) visible light photocrosslinking through formation of covalent bonds. This allows engineering of mechanically robust wet tissue adhesives, which is critical for treatment of two major PIDs, including peri-implant mucositis (PIM) and peri-implantitis (PI).

FIG. 34 depicts the synthesis process of the wet tissue bioadhesives by conjugation of dopamine to gelatin backbone and further methacryloyl functionalization of the polymer.

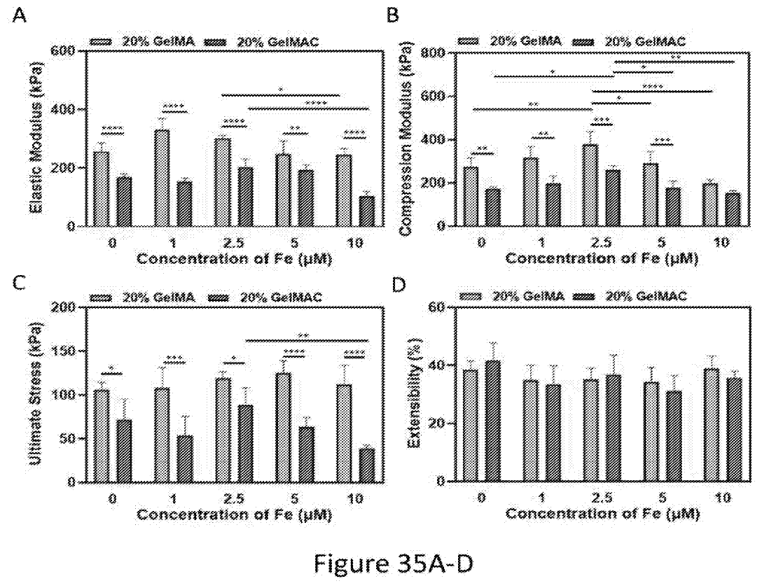

FIG. 35, comprising FIGS. 35A-D, depicts the physical characterization of the GelMAC bioadhesive. FIG. 35A depicts the elastic modulus of a GelMAC wet tissue bioadhesive hydrogel. FIG. 35B depicts the compressive modulus of a GelMAC wet tissue bioadhesive hydrogel. FIG. 35C depicts the ultimate stress of a GelMAC wet tissue bioadhesive hydrogel. FIG. 35D depicts the extensibility of a GelMAC wet tissue bioadhesive hydrogel.

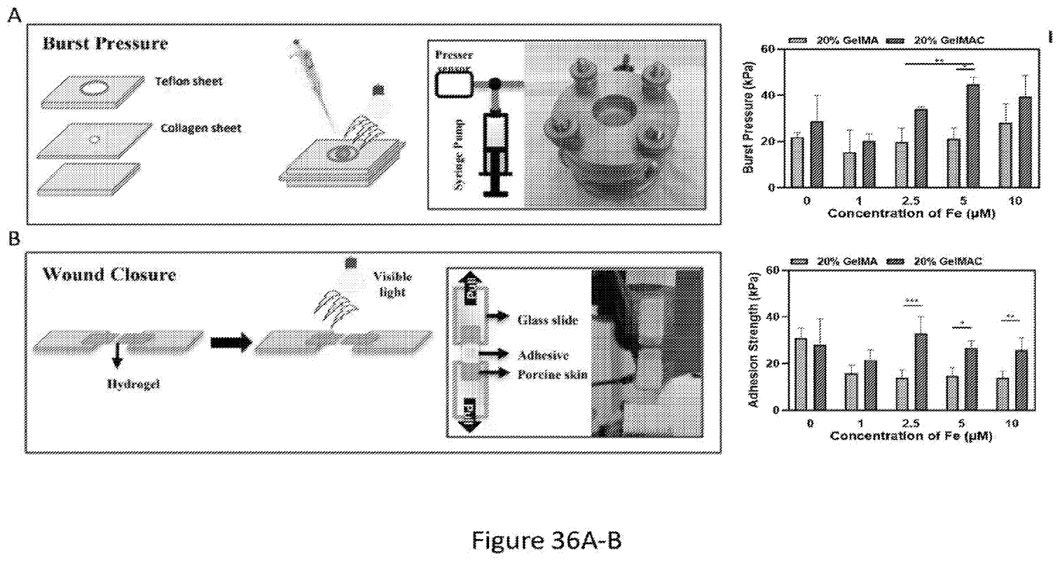

FIG. 36, comprising FIGS. 36A-B, depicts in vitro adhesion properties of the bioadhesive hydrogels. FIG. 36A depicts a burst pressure test. FIG. 36B depicts a wound closure test.

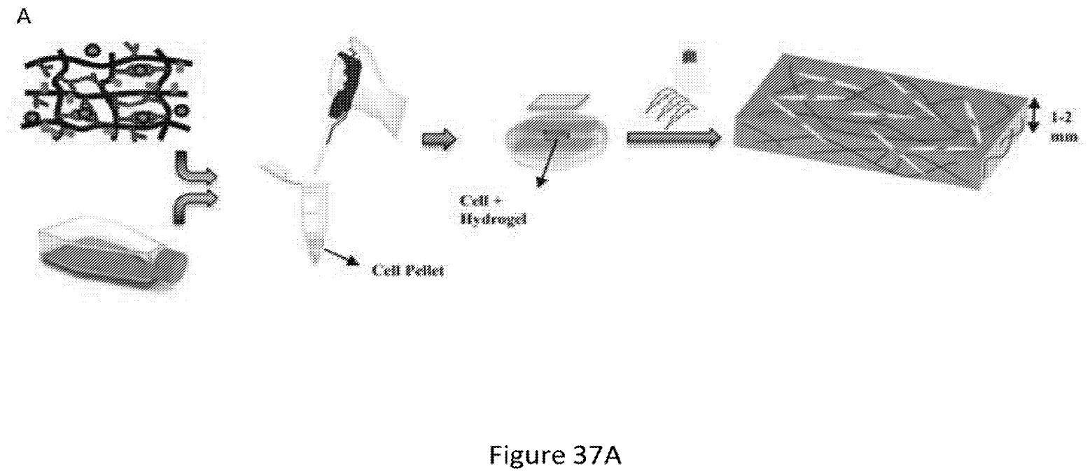

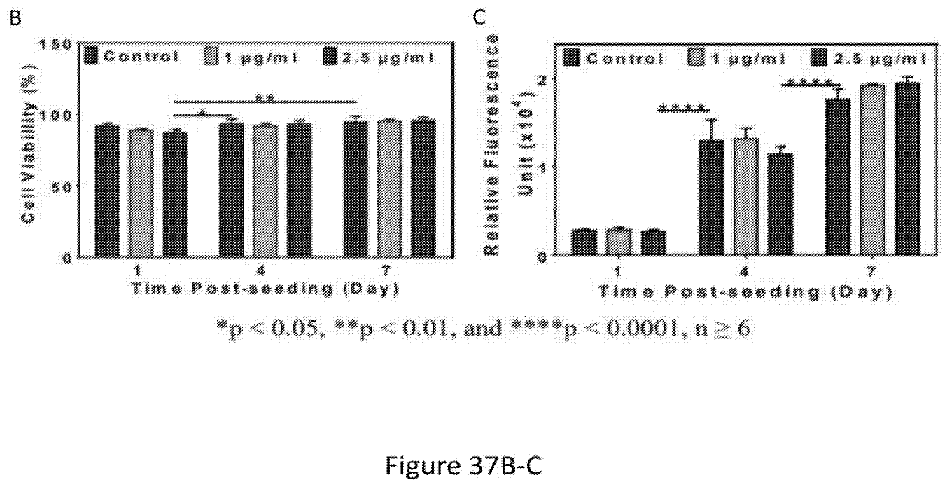

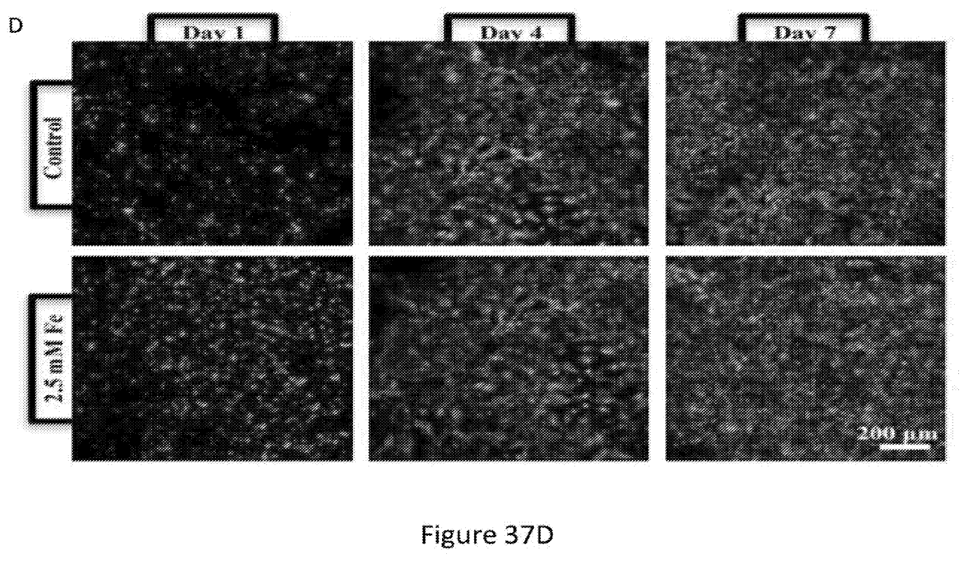

FIG. 37, comprising FIGS. 37A-D, depicts in vitro cytocompatibility of the bioadhesive hydrogels. FIG. 37A shows the method of 3D cell encapsulation in wet tissue adhesives. FIG. 37B depicts the quantification of viability of the cells encapsulated within the adhesive hydrogels. FIG. 37C depicts the metabolic activity of the cells encapsulated within the adhesive hydrogels. FIG. 37D depicts the representative images of Live/Dead assay for the cells encapsulated within the wet tissue adhesives.

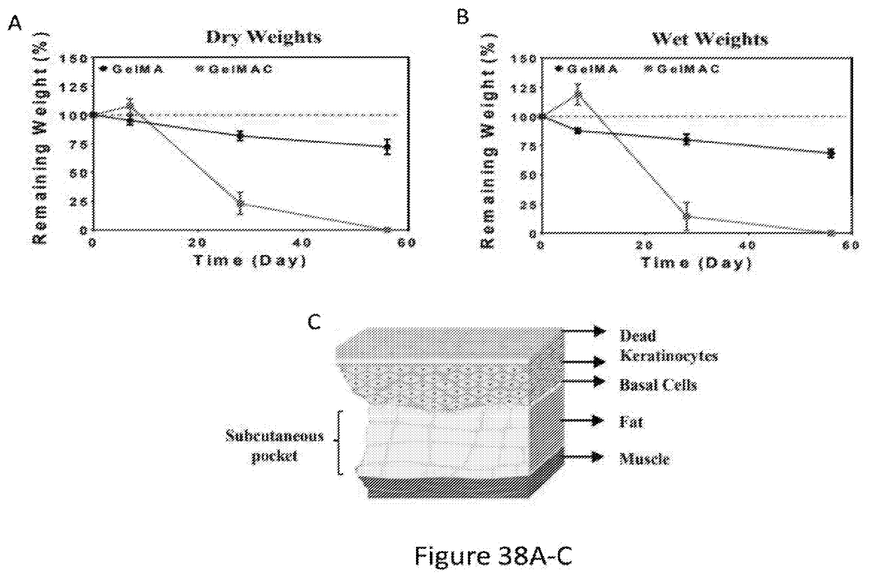

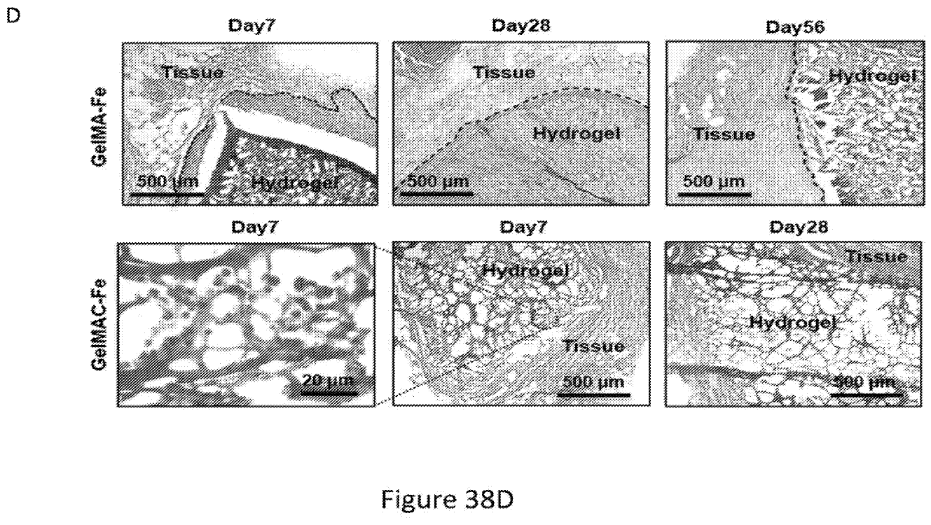

FIG. 38, comprising FIGS. 38A-D, depicts in vivo biodegradation and biocompatibility of composite hydrogels using a rat subcutaneous model (H&E staining). FIG. 38A depicts the biodegradation of wet tissue bioadhesives based on dry weight. FIG. 38B depicts the biodegradation of wet tissue bioadhesives based on wet weight. FIG. 38C represents a schematic of the location of the implanted samples in the rat subcutaneous pocket. FIG. 38D depicts representative H&E stained images from the cross sections of wet tissue bioadhesives explanted at days 7, 28, and 56.

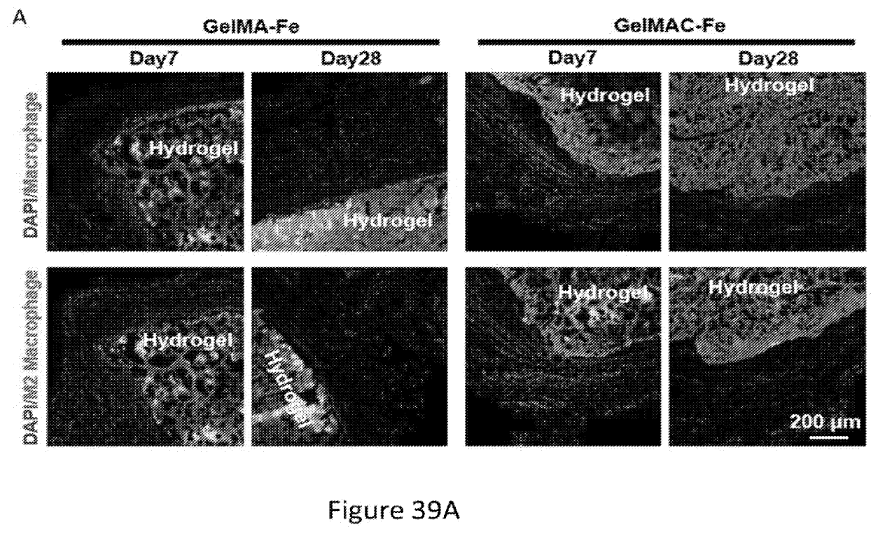

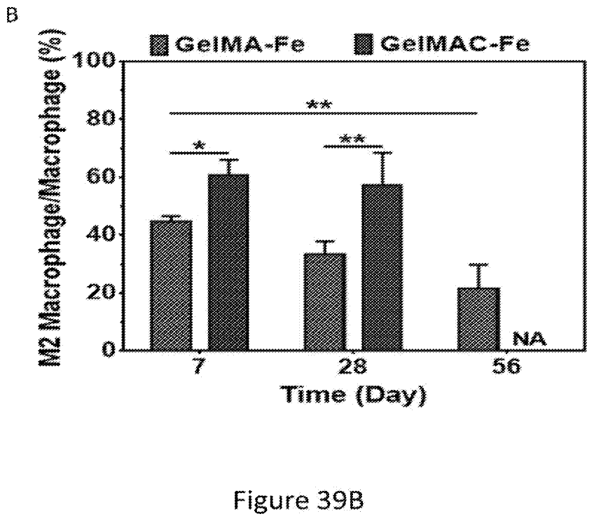

FIG. 39, comprising FIGS. 39A-B, depicts in vivo biocompatibility of composite hydrogels using a rat subcutaneous model (immunohistochemical analysis). FIG. 39A depicts immunofluorescent analysis of subcutaneously implanted wet tissue bioadhesive hydrogels, explanted at day 7, and day 28. The samples were stained for CD206 (M2 macrophages), and F4/80 (total macrophages). FIG. 39B depicts quantification of macrophage infiltration based on immunofluorescent analysis of subcutaneously implanted wet tissue bioadhesive hydrogels, explanted at days 7, 28, and 56.

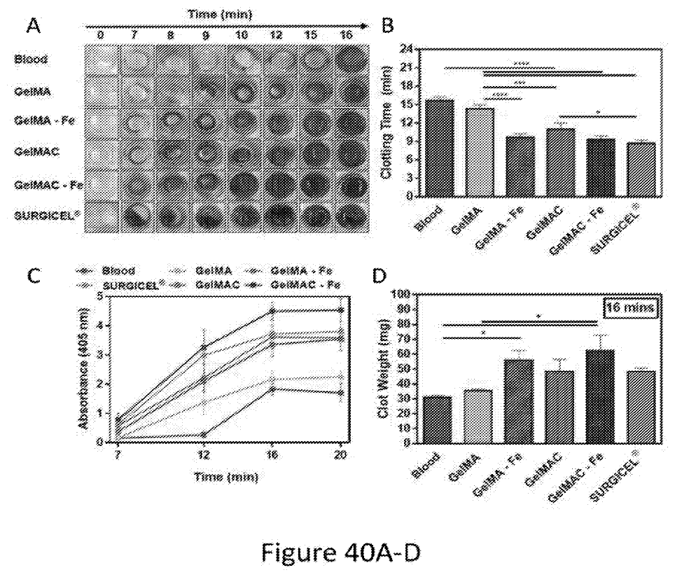

FIG. 40, comprising FIGS. 40A-D, depicts the hemostatic properties of the bioadhesive. FIG. 40A depicts the time-dependent clot formation of GelMA, GelMAC, GelMA-Fe, and GelMAC-Fe hydrogels compared with untreated blood (negative control) and SURGICEL® absorbable hemostat (positive control). FIG. 40B depicts the quantitative clot formation time of GelMA, GelMAC, GelMA-Fe, and GelMAC-Fe hydrogels compared with untreated blood (negative control) and SURGICEL® absorbable hemostat (positive control). FIG. 40C depicts the absorbance at 405 nm wavelength performed on clotted samples at various time points of 7, 12, 16, and 20 minutes for GelMA, GelMAC, GelMA-Fe, and GelMAC-Fe hydrogels compared with untreated blood (negative control) and SURGICEL® absorbable hemostat (positive control). FIG. 40D depicts the clot weight collected at a 16-minute time point for GelMA, GelMAC, GelMA-Fe, and GelMAC-Fe hydrogels compared with untreated blood (negative control) and SURGICEL® absorbable hemostat (positive control). (*p<0.05, ***p<0.001, ****p<0.0001 and n=4).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A hydrogel composition comprising a gelatin with crosslinkable groups, a crosslinking agent, an antimicrobial agent, and an osteoinductive agent.

2. The hydrogel composition of claim 1, wherein the crosslinked gelatin comprises crosslinkable groups selected from the group consisting of: methyl acrylate, ethyl acrylate, propyl acrylate, methyl methacrylate, ethyl methacrylate, methacryloyl, catechol, ethylene oxide, propylene oxide, and combinations thereof.

3. The hydrogel composition of claim 1, wherein the crosslinking agent comprises a photoinitiator selected from the group consisting of: 2-hydroxy-4′-(2-hydroxyethoxy)-2-methylpropiophenone; lithium phenyl-2,4,6-trimethylbenzoylphosphinate; 2,2-diethoxyacetophenone; triethanolamine; N-vinyl caprolactam; benzophenone; Eosin Y; and combinations thereof.

4. The hydrogel composition of claim 1, wherein the crosslinking agent comprises a metal²⁺ or metal³⁺ ion selected from the group consisting of: Fe²⁺, Fe³⁺, Ni²⁺, Zn²⁺, Cu²⁺, Ag²⁺, Au³⁺, Co²⁺, Co³⁺, Cr²⁺, Cr³⁺, Cd²⁺, Mn²⁺, Mg²⁺, Pd²⁺, Pt²⁺, Al³⁺, and combinations thereof.

5. The hydrogel composition of claim 1, wherein the antimicrobial agent comprises an antimicrobial peptide.

6. The hydrogel composition of claim 1, wherein the osteoinductive agent is selected from the group consisting of: silicate nanoparticles, calcium phosphate, calcium sulfate, bioglass, hydroxyapatite, demineralized bone matrix (DBM), and combinations thereof.

7. The hydrogel composition of claim 6, wherein the osteoinductive agent comprises silicate nanoparticles and wherein the silicate nanoparticles comprise laponite nanoparticles.

8. A method of making a hydrogel, the method comprising:
providing a solution comprising gelatin modified with crosslinkable groups;
providing a solution comprising a crosslinking agent;
mixing the solution comprising the gelatin modified with crosslinkable groups and the solution comprising the crosslinking agent to form a combined solution; and
crosslinking the combined solution.

9. The method of claim 8, wherein the step of providing a solution comprising a crosslinking agent further comprises the step of adding an antimicrobial agent to the solution.

10. The method of claim 8, wherein the step of mixing the solution comprising the gelatin modified with crosslinkable groups and the solution comprising the crosslinking agent to form a combined solution further comprises the step of adding an osteoinductive agent to the combined solution.

11. The method of claim 8, wherein the gelatin modified with crosslinkable groups is made by a method comprising the steps of:
providing a solution comprising gelatin; and
reacting the solution comprising gelatin with a compound comprising crosslinkable groups.

12. The method of claim 8, wherein the gelatin modified with crosslinkable groups is selected from the group consisting of gelatin modified with methacryloyl groups (GelMA), gelatin modified with catechol groups (GelMAC), and gelatin modified with both methacryloyl groups and catechol groups.

13. The method of claim 8, wherein the crosslinking agent is selected from the group consisting of 2-hydroxy-4′-(2-hydroxyethoxy)-2-methylpropiophenone; lithium phenyl-2,4,6-trimethylbenzoylphosphinate; 2,2-diethoxyacetophenone; triethanolamine; N-vinyl caprolactam; benzophenone; Eosin Y; Fe²⁺; Fe³⁺; Ni²⁺; Zn²⁺; Cu²⁺; Ag²⁺; Au³⁺; Co²⁺; Co³⁺; Cr²⁺; Cr³⁺; Cd²⁺; Mn²⁺; Mg²⁺; Pd²⁻; Pt²⁺; Al³⁺; and combinations thereof.

14. The method of claim 9, wherein the antimicrobial agent comprises an antimicrobial peptide.

15. A method of inhibiting microbial growth at the site of a dental implant, the method comprising:
applying a solution comprising a hydrogel precursor and an antimicrobial agent to one or more surfaces of a dental implant in the subject's mouth to form a coating; and
crosslinking the coating to form a hydrogel.

16. The method of claim 15, wherein the step of crosslinking the coating to form a hydrogel further comprises the step of crosslinking the coating by irradiating the coating with visible light.

17. The method of claim 15, wherein the step of crosslinking the coating to form a hydrogel further comprises the step of adhering the hydrogel to the dental implant in the subject's mouth.

18. The method of claim 15, wherein the microbial growth comprises microbial growth associated with peri-implant or periodontal diseases.

19. The method of claim 17, wherein the step of adhering the hydrogel to the dental implant in the subject's mouth further comprises the step of promoting bone growth at the site of the implant.

20. (canceled)

21. A method of promoting bone regrowth in a subject's mouth, the method comprising:
applying a hydrogel composition according to claim 1 to one or more defects in the subject's mandible or mouth as a bone graft; and
crosslinking the solution.
